(19) United States
(12) Patent Application Publication
(10) Pub. No.: US 2023/0102151 A1
(43) Pub. Date: Mar. 30, 2023

(54) METHODS FOR TREATING ATOPIC DERMATITIS BY ADMINISTERING AN IL-4R ANTAGONIST

(71) Applicants: REGENERON PHARMACEUTICALS, INC., Tarrytown, NY (US); SANOFI BIOTECHNOLOGY, Paris (FR)

(72) Inventors: Ashish BANSAL, White Plains, NY (US); John DAVIS, Tarrytown, NY (US); Laurent ECKERT, Paris (FR); Mohamed KAMAL, Tarrytown, NY (US)

(73) Assignees: REGENERON PHARMACEUTICALS, INC., Tarrytown, NY (US); SANOFI BIOTECHNOLOGY, Paris (FR)

(21) Appl. No.: 17/907,070

(22) PCT Filed: Mar. 26, 2021
(86) PCT No.: PCT/US2021/024419

§ 371 Date: Sep. 22, 2022

Related U.S. Application Data
(60) Provisional application No. 63/001,224, filed on Mar. 27, 2020.

(30) Foreign Application Priority Data
Jan. 28, 2021 (EP) 21315010.5

(51) Int. Cl. C07K 16/28; A61P 17/00; A61K 39/395

(52) U.S. Cl. CPC C07K 16/2866; A61P 17/00; A61K 39/3955; C07K 2317/21; C07K 2317/565; A61K 2039/545

(57) ABSTRACT

Methods for treating moderate-to-severe atopic dermatitis in a pediatric subject are provided. In one aspect, the methods comprise administering to the subject one or more doses of an interleukin-4 receptor (IL-4R) antagonist, such as an anti-IL-4R antibody or antigen-binding fragment thereof.

### CROSS-REFERENCE TO RELATED APPLICATIONS

This application is being filed on Mar. 26, 2021, as a PCT International Patent Application and claims priority to U.S. Provisional Patent Application No. 63/001,224, filed Mar. 27, 2020, and to European Patent Application No. 21315010.5, filed Jan. 28, 2021, the contents of each of which are incorporated by reference herein.

### FIELD OF THE INVENTION

The present disclosure relates to the use of interleukin-4 receptor (IL-4R) antagonists for treating atopic dermatitis.

### BACKGROUND

Atopic dermatitis (AD) is one of the most common skin disorders in infants and children, with onset under the age of 6 months in 45%, under the age of 1 year in 60%, and within the first 5 years in 89% of all cases (Mortz et al, Allergy 2015, 70:836-845; Kay et al, J Am Acad Dermatol 1994, 30:35-39). The prevalence has been estimated at 15-38% in children aged <5 years in the USA (Al-Naqeeb et al, J Am Board Fam Med 2019, 32:191-200) and 21.5% in children aged <2 years in Germany (Illi et al, J Allergy Clin Immunol 2004, 113:925-931).

AD markedly affects the quality of life (QoL) of both children and their families. In one study, nearly two-thirds of children with severe AD had moderately-to-highly impaired QoL (Ricci et al, Pediatr Allergy Immunol 2007, 18:245-249). In infants, the greatest impact of AD includes itching, sleep loss, mood and behavioral changes. In children, AD disturbs sleep, increases economic costs, parental fatigue and irritability, impairs daily activities and reduces leisure and family time as well as psychological and emotional well-being. See, e.g., Ramirez et al, JAMA Dermatol, 2019, 155:556-563.

The so-called “atopic march” in a subset of younger children, referring to the increased risk of developing asthma and/or allergic rhinitis in children with a history of AD and food allergies, suggests that AD may be an “entry point” for subsequent allergic disease. An estimated 60% of infants and young children with severe AD and 30% with mild AD develop asthma (Ricci et al, J Am Acad Dermatol 2006, 55:765-771). Despite the immune dysregulation shared by all atopic diseases, standard-of-care treatments have focused on long-term use of distinct topical products for skin, inhaled medications for asthma, nasal sprays for rhinitis, and oral antihistamines for itch. Management of these related conditions is often disjointed. Thus, there is a high need for a therapy that concurrently treats comorbid diseases in an effective manner.

Pharmacologic management of AD in children is primarily limited to topical corticosteroids (TCS). While clinically relevant side effects are unusual, younger children are at highest risk for systemic absorption, with potential growth retardation and hypothalamic pituitary axis suppression, due to their developmental status and higher ratio of body surface area (BSA) to weight. Non-corticosteroid alternatives, such as the topical calcineurin inhibitors (TCIs) tacrolimus and pimecrolimus, are used to minimize chronic TCS exposure in AD, but access to these medications is often limited by payers, based on labelling for children aged >2 years. Use of systemic corticosteroids is strongly discouraged in AD while other systemic immunosuppressants such as cyclosporine, methotrexate, azathioprine and mycophenolate mofetil have been used off-label despite significant potential side effects (e.g., growth retardation in children, Cushing's syndrome, hypertension, glucose intolerance, myopathy, osteonecrosis, glaucoma and cataracts). See, e.g., Lebwohl et al, 2019, J Drugs Dermatol, 18:122-129. Use of systemic immunosuppressants also carries the risk of rebound phenomenon, wherein symptoms of the disease may worsen significantly following cessation of treatment. Thus, in children with AD, there is a significant unmet need for a therapy with a favorable risk—benefit profile that can lead to rapid disease improvement.

### SUMMARY

In one aspect, methods for treating atopic dermatitis (AD) or improving an AD-associated parameter in a subject are provided. In some embodiments, the method comprises administering one or more doses of an interleukin-4 receptor (IL-4R) antagonist to a pediatric subject with moderate-to-severe or severe AD that is not adequately controlled by topical AD medications, wherein the subject is ≥6 months to <6 years of age. In some embodiments, the IL-4R antagonist is an anti-IL-4R antibody, or an antigen-binding fragment thereof.

In some embodiments, the method comprises:

(a) selecting a subject with moderate-to-severe or severe AD that is not adequately controlled by topical AD medications, wherein the subject is ≥6 months to <6 years of age; and

(b) administering to the subject one or more doses of an interleukin-4 receptor (IL-4R) antagonist, wherein the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of SEQ ID NO:7, and the LCDR3 comprises the amino acid sequence of SEQ ID NO:8.

In some embodiments, the subject is a subject with severe AD. In some embodiments, the subject is inadequately responsive to treatment with a topical corticosteroid (TCS) of medium or higher potency. In some embodiments, the subject previously was administered a systemic AD medication.

In some embodiments, at the onset of treatment the subject is aged ≥6 months to <2 years. In some embodiments, at the onset of treatment the subject is aged ≥2 to <6 years.

In some embodiments, the subject:

(i) has a baseline Investigator's Global Assessment (IGA) score=4;

(ii) has a baseline Eczema Area and Severity Index (EAST) score≥21; and/or

(iii) has a baseline Body Surface Area (BSA) affected by AD≥15%.

In some embodiments, the subject has at least one concurrent atopic or allergic condition. In some embodiments, the subject has a concurrent atopic or allergic condition selected from the group consisting of allergic rhinitis, asthma, food allergy, allergic conjunctivitis, hives, chronic rhinosinusitis, nasal polyps, and eosinophilic esophagitis.

In some embodiments, the IL-4R antagonist is subcutaneously administered at a dose of 3 mg/kg. In some embodiments, the IL-4R antagonist is subcutaneously administered at a dose of 6 mg/kg. In some embodiments, the method comprises administering multiple doses of the IL-4R antagonist. In some embodiments, the IL-4R antagonist is administered once a week or once every two weeks.

In some embodiments, the subject is administered the IL-4R antagonist in combination with a topical medication (e.g., a topical corticosteroid (TCS) or a topical nonsteroidal medication). In some embodiments, the subject is administered the IL-4R antagonist in combination with a TCS. In some embodiments, the TCS is a medium-potency TCS. In some embodiments, the TCS is a low-potency TCS. In some embodiments, treatment with the IL-4R antagonist reduces the amount of TCS that is administered to the subject relative to baseline.

In some embodiments, treatment with the IL-4R antagonist results in a reduction in the level of one or more type 2 inflammatory biomarkers in the subject relative to a baseline value. In some embodiments, treatment with the IL-4R antagonist results in a reduction in the level of serum TARC and/or serum total IgE in the subject relative to a baseline value, e.g., a reduction of at least 20%, 25%, 30%, 35%, 40%, 45%, 50% or more relative to a baseline value.

In some embodiments, treatment with the IL-4R antagonist results improves an AD-associated parameter that is selected from:

(i) a reduction from baseline in IGA score to achieve an IGA score of 0 or 1 by week 4 after administration of the first dose of the IL-4R antagonist;

(ii) reduction of at least 50% from baseline in an EASI score (EASI-50) by week 3 after administration of the first dose of the IL-4R antagonist;

(iii) a reduction of at least 75% from baseline in an EASI score (EASI-75) by week 3 after administration of the first dose of the IL-4R antagonist;

(iv) a reduction in percentage of BSA affected by AD to less than 40% of BSA by week 3 after administration of the first dose of the IL-4R antagonist; and

(v) a reduction of at 35% from baseline in BSA affected by AD by week 3 after administration of the first dose of the IL-4R antagonist.

In some embodiments, one or more of the AD-associated parameters are assessed by a caregiver. In some embodiments, an improvement in one or more AD-associated parameters is based on a caregiver reported assessment. In some embodiments, the caregiver reported assessment is a caregiver-reported Peak Pruritus numerical rating scale (NRS). In some embodiments, treatment with the IL-4R antagonist results in an improvement in caregiver-reported Peak Pruritus NRS score.

In some embodiments, treatment with the IL-4R antagonist results in an improvement in itch (e.g., as measured by change in NRS score, or by change in SCORAD score or a component thereof). In some embodiments, a baseline level of itch and/or improvement in itch is assessed by a caregiver. In some embodiments, the improvement in itch is assessed by caregiver-reported Peak Pruritus NRS score.

In some embodiments, the IL-4R antagonist is an anti-IL-4R antibody, or an antigen-binding fragment thereof, that specifically binds IL-4R. In some embodiments, the anti-IL-4R antibody or antigen-binding fragment thereof comprises a heavy chain variable region (HCVR) comprising the amino acid sequence of SEQ ID NO:1 and a light chain variable region (LCVR) comprising the amino acid sequence of SEQ ID NO:2. In some embodiments, the anti-IL-4R antibody comprises a heavy chain comprising the amino acid sequence of SEQ ID NO:9 and a light chain comprising the amino acid sequence of SEQ ID NO:10. In some embodiments, the IL-4R antagonist is dupilumab or a bioequivalent thereof.

In some embodiments, the IL-4R antagonist (e.g., an anti-IL-4R antibody, or antigen-binding fragment thereof, as disclosed herein) is contained in a container selected from the group consisting of a glass vial, a syringe, a pre-filled syringe, a pen delivery device, and an autoinjector. In some embodiments, the IL-4R antagonist is contained in a pre-filled syringe. In some embodiments, the pre-filled syringe is a single-dose pre-filled syringe. In some embodiments, the IL-4R antagonist is contained in an autoinjector. In some embodiments, the IL-4R antagonist is contained in a pen delivery device (e.g., a pre-filled pen).

Other embodiments will be apparent from a review of the ensuing detailed description.

### BRIEF DESCRIPTION OF THE FIGURES

FIGS. 1A-1B. Pharmacokinetics of single-dose dupilumab over time in the two age cohorts (≥6 months to <2 years and ≥2 years to <6 years). (1A) Mean (SD) concentrations by dose group and nominal time on log-linear scale. Samples below the LLoQ were set to LLoQ/2. In the older cohort, dupilumab was undetectable at all time points in 1 patient who received the 3 mg/kg dose; this patient was excluded from all summary plots and descriptive statistics. (1B) Mean (SD) concentrations by dose group and nominal time on linear scale. Samples below the LLoQ were set to 0. In the older cohort, dupilumab was undetectable at all time points in 1 patient who received the 3 mg/kg dose; this patient was excluded from all summary plots and descriptive statistics. LLoQ, lower limit of quantitation; n=number of patients; SD, standard deviation.

FIGS. 2A-2E. Efficacy outcomes in the two age cohorts (≥6 months to <2 years and ≥2 years to <6 years): (2A) mean percentage change from baseline to Week 4 in EASI; (2B) mean percentage change from baseline to Week 4 in SCORAD score; (2C) proportions of patients with EASI-50; (2D) proportions of patients with EASI-75 from baseline to Week 4; (2E) mean percentage change from baseline to Week 4 in caregiver-reported Peak Pruritus NRS. EASI, Eczema Area and Severity Index; EASI-50/-75, ≥50%/≥75% improvement from baseline in EASI; NRS, numerical rating scale; SCORAD, SCORing Atopic Dermatitis; SD, standard deviation.

### DETAILED DESCRIPTION

Before the present invention is described, it is to be understood that the invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

As used herein, the terms “treat,” “treating,” or the like, mean to alleviate symptoms, eliminate the causation of symptoms either on a temporary or permanent basis, or to prevent or slow the appearance of symptoms of the named disorder or condition.

“Atopic dermatitis” or “AD”, as used herein, means an inflammatory skin disease characterized by intense pruritus (e.g., severe itch) and by scaly and dry eczematous lesions. The term “atopic dermatitis” includes, but is not limited to, AD caused by or associated with epidermal barrier dysfunction, allergy (e.g., allergy to certain foods, pollen, mold, dust mite, animals, etc.), radiation exposure, and/or asthma. The present disclosure encompasses methods to treat patients with moderate-to-severe or severe AD. As used herein, “moderate-to-severe AD” is characterized by intensely pruritic, widespread skin lesions that are often complicated by persistent bacterial, viral or fungal infections. Moderate-to-severe AD also includes chronic AD in patients. In many cases, the chronic lesions include thickened plaques of skin, lichenification and fibrous papules. Patients affected by moderate-to-severe AD also, in general, have more than 20% of the body's skin affected, or 10% of skin area in addition to involvement of the eyes, hands and body folds. Moderate-to-severe AD is also considered to be present in patients who require frequent treatment with topical corticosteroids. A patient may also be said to have moderate-to-severe AD when the patient is resistant or refractory to treatment by either a topical corticosteroid or a calcineurin inhibitor. As used herein, “severe AD” is characterized by the presence of widespread skin lesions, unremitting itching, or physically or emotionally disabling disease that significantly compromises a patient's quality of life. In some cases, patients with severe AD also exhibits one or more symptoms such as excoriation, extensive skin thickening, bleeding, oozing, and/or cracking of skin, and alteration of pigmentation. In some embodiments, severe AD is refractory to treatment by a topical therapy (e.g., a topical corticosteroid, calcineurin inhibitor, or crisaborole).

As used herein, the term “subject in need thereof” refers to a human or a non-human animal having AD (e.g., moderate-to-severe AD or severe AD). In some embodiments, the term “a subject in need thereof” refers to patients with moderate-to-severe or severe AD, wherein the patient is ≥6 months and <6 years of age, e.g., a subject who is ≥6 months and <2 years of age or a subject who is ≥2 and <6 years of age. The terms “subject” and “patient” are used interchangeably herein.

In some embodiments, the term “subject in need thereof” includes patients with moderate-to-severe or severe AD who are ≥6 months and <6 years of age and who have received prior treatment with systemic therapy. As used herein, the term “systemic therapy” refers to systemically administered therapeutic agents (e.g., orally administered corticosteroids). The term includes systemic immunosuppressant or immunomodulatory agents. In the context of the present disclosure, the term “systemic immunosuppressant” includes, but is not limited to, cyclosporine A, methotrexate, mycophenolate mofetil, azathioprine, systemic or oral corticosteroids, and interferon-gamma. In certain embodiments, the term also includes immunobiologics such as tumor necrosis factor alpha (TNFα) inhibitors (e.g., an anti-TNFα antibody such as infliximab), CD11a inhibitors (e.g., an anti-CD11a antibody such as efalizumab), IgE inhibitors (e.g., omalizumab), CD20 inhibitors (e.g., rituximab). Systemic therapy including systemic immunosuppressants may be used for short-term treatment of flares or as a temporary measure to control disease, but their use is limited by significant side-effects, e.g., growth retardation in children, Cushing's syndrome, hypertension, glucose intolerance, myopathy, osteonecrosis, glaucoma and cataracts. Use of systemic immunosuppressants also carries the risk of rebound phenomenon, wherein symptoms of the disease may worsen significantly following cessation of treatment. In certain embodiments, the terms “systemic therapy”, “systemic therapeutic agent” and “systemic immunosuppressant” have been used interchangeably throughout this disclosure.

The term “TCS,” as used herein, includes group I, group II, group III and group IV topical corticosteroids. According to the Anatomical Therapeutic Classification System of World Health Organization, the corticosteroids are classified as weak (group I), moderately potent (Group II) and potent (Group III) and very potent (Group IV), based on their activity as compared to hydrocortisone. Group IV TCS (very potent) are up to 600 times as potent as hydrocortisone and include clobetasol propionate and halcinonide. Group III TCS (potent) are 50 to 100 times as potent as hydrocortisone and include, but are not limited to, betamethasone valerate, betamethasone dipropionate, diflucortolone valerate, hydrocortisone-17-butyrate, mometasone furoate, and methylprednisolone aceponate. Group II TCS (moderately potent; also referred to interchangeably herein as “medium potency”) are 2 to 25 times as potent as hydrocortisone and include, but are not limited to, clobetasone butyrate, and triamcinolone acetonide. Group I TCS (mild; also referred to interchangeably herein as “low potency”) includes hydrocortisone.

Although any methods and materials similar or equivalent to those described herein can be used in the practice of the disclosure, the typical methods and materials are now described. All publications mentioned herein are incorporated herein by reference in their entirety.

### Therapeutic Methods

In one aspect, methods for treating atopic dermatitis (AD) or improving an AD-associated parameter in a subject are provided. In some embodiments, the methods comprise administering to a subject having moderate-to-severe or severe AD, wherein the subject is ≥6 months and <6 years of age, one or more doses of an interleukin-4 receptor (IL-4R) antagonist. In some embodiments, the IL-4R antagonist is administered concomitantly with topical therapy for AD, such as a topical corticosteroid (TCS) or a topical nonsteroidal medication (e.g., a calcineurin inhibitor or crisaborole). In some embodiments, the subject is ≥6 months and <1 year of age. In some embodiments, the subject is ≥6 months and <2 years of age. In some embodiments, the subject is ≥1 and <2 years of age. In some embodiments, the subject is ≥2 and <4 years of age. In some embodiments, the subject is ≥4 and <6 years of age. In some embodiments, the subject is ≥3 and <6 years of age. In some embodiments, the subject is ≥2 and <6 years of age. In some embodiments, the subject is ≥1 and <6 years of age.

In some embodiments, a subject to be treated according to the methods disclosed herein is a subject ≥6 months and <6 years of age (e.g., a subject ≥6 months and <2 years of age or a subject ≥2 and <6 years of age) who has severe AD that is inadequately responsive to topical therapies (e.g., TCS with or without topical calcineurin inhibitors (TCIs)) or for whom topical therapy is inadvisable (e.g., due to adverse side effects or safety risks). In some embodiments, the subject has a documented history of inadequate response to a sufficient course of outpatient treatment with topical AD medication(s). As used herein, “inadequate response” refers to a failure to achieve and maintain remission or a low disease activity state (comparable to Investigator's Global Assessment [IGA] 0=clear to 2=mild) despite treatment for at least 28 days with a topical therapy (e.g., a regimen of TCS of medium to high potency, TCI as appropriate). In some embodiments, a subject has an “inadequate response” if the patient has received documented systemic treatment for AD.

In some embodiments, treatment with an IL-4R antagonist improves, alleviates, or reduces one or more symptoms of AD in a subject, including but not limited to pruritus (i.e., itchiness), xerosis (skin dryness), eczematous lesions, erythema, papulation, edema, oozing/crusting, excoriation, lichenification, sleep disturbance, anxiety, and depression.

In some embodiments, treatment with an IL-4R antagonist improves one or more AD-associated parameters in a subject. Examples of “AD-associated parameters” include, but are not limited to: (a) Investigators Global Assessment (IGA); (b) Body Surface Area Involvement of Atopic Dermatitis (BSA); (c) Eczema Area and Severity Index (EASI); (d) SCORAD; (e) 5-D Pruritus Scale; and (f) Pruritus Numeric Rating Scale (NRS). An “improvement in an AD-associated parameter” means a decrease from baseline of one or more of IGA, BSA, EASI, SCORAD, 5-D Pruritus Scale, NRS/worst itch score, patient global impression of disease, patient global impression of change, Children's Dermatology Life Quality Index (CDLQI), Patient Oriented Eczema Measure (POEM), Dermatitis Family Index (DFI) score, or Patient-Reported Outcomes Measurement Information System (PROMIS) anxiety and/or depression score. The term “baseline,” as used with respect to an AD-associated parameter, means the numerical value of the AD-associated parameter for a subject prior to or at the time of administration of a pharmaceutical composition as disclosed herein.

To determine whether an AD-associated parameter has “improved,” the parameter is quantified at baseline and at one or more time points after administration of the pharmaceutical composition of the present disclosure. For example, an AD-associated parameter may be measured at day 1, day 2, day 3, day 4, day 5, day 6, day 7, day 8, day 9, day 10, day 11, day 12, day 14, day 15, day 22, day 25, day 29, day 36, day 43, day 50, day 57, day 64, day 71, day 85; or at the end of week 1, week 2, week 3, week 4, week 5, week 6, week 7, week 8, week 9, week 10, week 11, week 12, week 13, week 14, week 15, week 16, week 17, week 18, week 19, week 20, week 21, week 22, week 23, week 24, or longer, after the initial treatment with a pharmaceutical composition of the present disclosure. The difference between the value of the parameter at a particular time point following initiation of treatment and the value of the parameter at baseline is used to establish whether there has been an “improvement” (e.g., a decrease) in the AD associated parameter. AD-associated parameters are described in US Patent Publication No. US 2014/0072583, incorporated herein in its entirety.

In some embodiments, an AD-associated parameter is assessed by a caregiver. In some embodiments, a parameter is quantified at baseline and at one or more time points after administration of the pharmaceutical composition based on caregiver assessment of the AD-associated parameter. In some embodiments, a caregiver reported assessment is used to assess an AD-associated parameter in a patient ≥6 months and <6 years of age, e.g., a patient ≥6 months and <4 years of age or a patient ≥6 months and <2 years of age. In some embodiments, a caregiver reported assessment is used to assess improvement in peak pruritus NRS score, patient global impression of disease, patient global impression of change, Children's Dermatology Life Quality Index (CDLQI), Patient Oriented Eczema Measure (POEM), Dermatitis Family Index (DFI) score, or Patient-Reported Outcomes Measurement Information System (PROMIS) anxiety and/or depression score. In some embodiments, improvement in itch is determined based on a caregiver reported assessment. In some embodiments, improvement in itch is assessed by caregiver reported peak pruritus NRS score.

In some embodiments, treatment with an IL-4R antagonist according to the methods of the present disclosure results in an improvement in IGA score for the subject relative to baseline. Methods for determining an IGA score for a subject are described in the Examples section below. In some embodiments, a subject to be treated has a baseline IGA score ≥3 (e.g., an IGA score of 3 or an IGA score of 4). In some embodiments, treatment with an IL-4R antagonist results in a reduction from baseline in IGA score (e.g., from a baseline IGA score ≥3 or a baseline IGA score=4) of at least 1 point by week 3, week 4, week 8, week 12, or week 16 after administration of the first dose of the IL-4R antagonist. In some embodiments, treatment with an IL-4R antagonist results in a reduction from baseline (e.g., from an IGA score ≥3 or an IGA score=4) to an IGA score of 0 or 1 by week 3, week 4, week 8, week 12, or week 16 after administration of the first dose of the IL-4R antagonist.

In some embodiments, treatment with an IL-4R antagonist according to the methods of the present disclosure results in an improvement in an EASI score for a subject relative to baseline. Methods for determining an EASI score for a subject are described in the Examples section below. In some embodiments, a subject to be treated has a baseline EASI score of ≥21 (e.g., an EASI score ≥30). In some embodiments, treatment with an IL-4R antagonist results in a reduction of at least 30%, at least 40%, at least 50%, at least 60%, at least 70%, at least 75%, at least 80%, or at least 90% from baseline in an EASI score by week 3, week 4, week 8, week 12, or week 16 after administration of the first dose of the IL-4R antagonist. In some embodiments, treatment with an IL-4R antagonist results in the subject achieving an EASI-75 response (i.e., a ≥75% improvement from baseline) by week 3, week 4, week 8, week 12, or week 16 after administration of the first dose of the IL-4R antagonist. In some embodiments, treatment with an IL-4R antagonist results in the subject achieving an EASI-50 response (i.e., a ≥50% improvement from baseline) by week 3, week 4, week 8, week 12, or week 16 after administration of the first dose of the IL-4R antagonist.

In some embodiments, treatment with an IL-4R antagonist according to the methods of the present disclosure results in an improvement in a BSA score for a subject relative to baseline. Methods for determining a BSA score for a subject are described in the Examples section below. In some embodiments, a subject to be treated has a baseline BSA score of ≥15% (e.g., ≥20%, ≥30%, ≥40%, ≥50%, ≥75%, or ≥90%). In some embodiments, a subject to be treated has a baseline BSA score of ≥50%. In some embodiments, treatment with an IL-4R antagonist results in a reduction of at least 10%, at least 20%, at least 30%, at least 40%, at least 50% or more from baseline in percent BSA that is affected by AD by week 3, week 4, week 8, week 12, or week 16 after administration of the first dose of the IL-4R antagonist.

In some embodiments, treatment with an IL-4R antagonist according to the methods of the present disclosure results in an improvement in a pruritus score, such as a “worst itch scale” score, also referred to herein as a Peak Pruritus Numeric Rating Scale (NRS) score, for a subject relative to baseline. Methods for determining a pruritus score are described in the Examples section below. In some embodiments, a subject to be treated has a baseline worst itch score weekly average score for maximum itch intensity that is ≥4 (e.g., ≥7). In some embodiments, treatment with an IL-4R antagonist results in a reduction of ≥3 points (e.g., ≥4 points) of a weekly average of a daily pruritus score (e.g., worst itch score) from baseline by week 3, week 4, week 8, week 12, or week 16 after administration of the first dose of the IL-4R antagonist.

In some embodiments, treatment with an IL-4R antagonist according to the methods of the present disclosure results in an improvement in a SCORAD score for the subject relative to baseline. Methods for determining a SCORAD score for a subject are described in the Examples section below. In some embodiments, a subject to be treated has a baseline SCORAD score ≥40 (e.g., a SCORAD score ≥50, ≥60, or ≥70). In some embodiments, treatment with an IL-4R antagonist results in a reduction in SCORAD score of at least 20%, at least 30%, at least 40%, at least 50%, at least 60%, at least 70%, at least 75%, at least 80%, or at least 90% from baseline by week 3, week 4, week 8, week 12, or week 16 after administration of the first dose of the IL-4R antagonist.

In some embodiments, treatment with an IL-4R antagonist enhances the efficacy and/or safety of a topical therapy for AD. As used herein, a topical therapy (e.g., TCS) regimen is “enhanced” if one or more of the following outcomes or phenomena are observed or achieved in a subject: (1) the amount of the topical agent (e.g., TCS) that is concomitantly administered is reduced; (2) the number of days in which the topical agent (e.g., TCS) is concomitantly administered is reduced; (3) the patient is administered a lower potency of the topical agent (e.g., the patient is switched from a medium-potency TCS to a low-potency TCS); (4) there is a reduction in or elimination of one or side effects due to the topical agent (e.g., TCS); or (5) there is a reduction in toxicity due to the topical agent (e.g., TCS). In some embodiments, the amount of the topical agent (e.g., TCS) that is concomitantly administered to the subject is decreased by at least 20%, 30%, 40%, 50%, 60%, 70%, 80% or more as compared to a baseline value for the subject or as compared to a subject that is not administered an IL-4R inhibitor. In some embodiments, treatment with an IL-4R antagonist allows for concomitant treatment with the topical agent (e.g., TCS) to be tapered off or discontinued.

### Interleukin-4 Receptor Antagonists

In some embodiments, the methods of the present disclosure comprise administering to a subject in need thereof (e.g., a subject having moderate-to-severe AD who is ≥6 months and <6 years of age, such as a subject ≥6 months and <2 years of age or a subject ≥2 and <6 years of age) an interleukin-4 receptor (IL-4R) antagonist or a pharmaceutical composition comprising an IL-4R antagonist. As used herein, an “IL-4R antagonist ” (also referred to herein as an “IL-4R inhibitor”, an “IL-4R blocker,” or an “IL-4Rα antagonist”) is any agent that binds to or interacts with IL-4Rα or an IL-4R ligand, and inhibits or attenuates the normal biological signaling function of a type 1 and/or a type 2 IL-4 receptor. Human IL-4Rα has the amino acid sequence of SEQ ID NO:11. A type 1 IL-4 receptor is a dimeric receptor comprising an IL-4Rα chain and a γc chain. A type 2 IL-4 receptor is a dimeric receptor comprising an IL-4Rα chain and an IL-13Rα1 chain. Type 1 IL-4 receptors interact with and are stimulated by IL-4, while type 2 IL-4 receptors interact with and are stimulated by both IL-4 and IL-13. Thus, the IL-4R antagonists that can be used in the methods of the present disclosure may function by blocking IL-4-mediated signaling, IL-13-mediated signaling, or both IL-4- and IL-13-mediated signaling. The IL-4R antagonists of the present disclosure may thus prevent the interaction of IL-4 and/or IL-13 with a type 1 or type 2 receptor.

Non-limiting examples of categories of IL-4R antagonists include small molecule IL-4R inhibitors, anti-IL-4R aptamers, peptide-based IL-4R inhibitors (e.g., “peptibody” molecules), “receptor-bodies” (e.g., engineered molecules comprising the ligand-binding domain of an IL-4R component), and antibodies or antigen-binding fragments of antibodies that specifically bind human IL-4Rα. As used herein, IL-4R antagonists also include antigen-binding proteins that specifically bind IL-4 and/or IL-13.

### Anti-IL-4Rα Antibodies and Antigen-Binding Fragments Thereof

In certain exemplary embodiments of the present disclosure, the IL-4R antagonist is an anti-IL-4Rα antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L}1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In some embodiments, the FRs of the anti-IL-4R antibody (or antigen-binding portion thereof) are identical to the human germline sequences. In some embodiments, one or more FRs of the anti-IL-4R antibody (or antigen-binding portion thereof) are naturally or artificially modified.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed by the term “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) V_{H}-C_{H}1; (ii) V_{H}-C_{H}2; (iii) V_{H}-C_{H}3; (iv) V_{H}-C_{H}1-C_{H}2; (V) V_{H}-C_{H}1-C_{H}2-C_{H}3; (vi) V_{H}-C_{H}2-C_{H}3; (vii) V_{H}-C_{L}; (viii) V_{L}-C_{H}1; (ix) V_{L}-C_{H}2; (x) V_{L}-C_{H}3; (xi) V_{L}-C_{H}1-C_{H}2; (xii) V_{L}-C_{H}1-C_{H}2-C_{H}3; (xiii) V_{L}-C_{H}2-C_{H}3; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

The constant region of an antibody is important in the ability of an antibody to fix complement and mediate cell-dependent cytotoxicity. Thus, in some embodiments the isotype of an antibody may be selected on the basis of whether it is desirable for the antibody to mediate cytotoxicity.

The term “antibody,” as used herein, also includes multispecific (e.g., bispecific) antibodies. A multispecific antibody or antigen-binding fragment of an antibody will typically comprise at least two different variable domains, wherein each variable domain is capable of specifically binding to a separate antigen or to a different epitope on the same antigen. Any multispecific antibody format may be adapted for use in the context of an antibody or antigen-binding fragment of an antibody of the present disclosure using routine techniques available in the art. For example, in some embodiments the methods of the present disclosure comprise the use of bispecific antibodies wherein one arm of an immunoglobulin is specific for IL-4Rα or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab² bispecific formats (see, e.g., Klein et al. 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L3} regions of the recombinant antibodies are sequences that, while derived from and related to human germline V_{H} and V_{L} sequences, may not naturally exist within the human antibody germline repertoire in vivo.

An “isolated antibody” refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

According to certain embodiments, the antibodies used in the methods of the present disclosure specifically bind IL-4Rα. The term “specifically binds,” as used herein, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. In some embodiments, an antibody that “specifically binds” IL-4Rα binds to IL-4Rα or a portion thereof with an equilibrium dissociation constant (KD) of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 1 nM, less than about 0.5 nM, less than about 0.25 nM, less than about 0.1 nM or less than about 0.05 nM, as measured in a surface plasmon resonance assay (e.g., BIAcore™, Biacore Life Sciences division of GE Healthcare, Piscataway, N.J.). In some embodiments, an antibody that specifically binds to a target antigen (e.g., IL-4Rα) can also specifically bind to another antigen, e.g., an ortholog of the target antigen. For example, in some embodiments, an isolated antibody that specifically binds human IL-4Rα exhibits cross-reactivity to other antigens, such as IL-4Rα molecules from other (non-human) species.

In some embodiments, the IL-4R antagonist is an anti-IL-4Rα antibody, or antigen-binding fragment thereof, comprising a heavy chain variable region (HCVR), light chain variable region (LCVR), and/or complementarity determining regions (CDRs) comprising any of the amino acid sequences of the anti-IL-4R antibodies as set forth in U.S. Pat. No. 7,608,693, incorporated by reference herein. In some embodiments, the IL-4R antagonist is an anti-IL-4Rα antibody or antigen-binding fragment thereof that comprises the heavy chain complementarity determining regions (HCDRs) of a heavy chain variable region (HCVR) comprising the amino acid sequence of SEQ ID NO:1 and the light chain complementarity determining regions (LCDRs) of a light chain variable region (LCVR) comprising the amino acid sequence of SEQ ID NO:2. In some embodiments, the IL-4R antagonist is an anti-IL-4Rα antibody or antigen-binding fragment thereof that comprises three HCDRs (HCDR1, HCDR2 and HCDR3) and three LCDRs (LCDR1, LCDR2 and LCDR3), wherein the HCDR1 comprises the amino acid sequence of SEQ ID NO:3; the HCDR2 comprises the amino acid sequence of SEQ ID NO:4; the HCDR3 comprises the amino acid sequence of SEQ ID NO:5; the LCDR1 comprises the amino acid sequence of SEQ ID NO:6; the LCDR2 comprises the amino acid sequence of SEQ ID NO:7; and the LCDR3 comprises the amino acid sequence of SEQ ID NO:8.

In some embodiments, the anti-IL-4R antibody or antigen-binding fragment thereof comprises the HCDR1, HCDR2, HCDR3, LCDR1, LCDR2, and LCDR3 of SEQ ID NOs:3, 4, 5, 6, 7, and 8, respectively, and further comprises an HCVR having at least 85% sequence identity (e.g., at least 90%, 91%, 92%, 93%, 94%, 95%, 96%, 97%, 98%, or 99% sequence identity) to the amino acid sequence of SEQ ID NO:1 and an LCVR having at least 85% sequence identity (e.g., at least 90%, 91%, 92%, 93%, 94%, 95%, 96%, 97%, 98%, or 99% sequence identity) to the amino acid sequence of SEQ ID NO:2. In some embodiments, the anti-IL-4R antibody or antigen-binding fragment thereof comprises an HCVR comprising SEQ ID NO:1 and an LCVR comprising SEQ ID NO:2.

In some embodiments, the anti-IL-4R antibody comprises a heavy chain comprising the amino acid sequence of SEQ ID NO:9. In some embodiments, the anti-IL-4R antibody comprises a light chain comprising the amino acid sequence of SEQ ID NO:10.

An exemplary antibody comprising a heavy chain comprising the amino acid sequence of SEQ ID NO:9 and a light chain comprising the amino acid sequence of SEQ ID NO:10 is the fully human anti-IL-4R antibody known as dupilumab. According to certain exemplary embodiments, the methods of the present disclosure comprise the use of dupilumab. As used herein, “dupilumab” also includes bioequivalents of dupilumab. The term “bioequivalent,” as used herein with reference to dupilumab, refers to anti-IL-4R antibodies or IL-4R-binding proteins or fragments thereof that are pharmaceutical equivalents or pharmaceutical alternatives whose rate and/or extent of absorption do not show a significant difference with that of dupilumab when administered at the same molar dose under similar experimental conditions, either single dose or multiple dose. In some embodiments, the term refers to antigen-binding proteins that bind to IL-4R which do not have clinically meaningful differences with dupilumab in their safety, purity and/or potency.

Other anti-IL-4Rα antibodies that can be used in the context of the methods of the present disclosure include, e.g., the antibody referred to and known in the art as AMG317 (Corren et al., 2010, Am J Respir Crit Care Med., 181(8):788-796), or MEDI 9314, or any of the anti-IL-4Rα antibodies as set forth in U.S. Pat. No. 7,186,809, U.S. Pat. No. 7,605,237, U.S. Pat. No. 7,638,606, U.S. Pat. No. 8,092,804, U.S. Pat. No. 8,679,487, U.S. Pat. No. 8,877,189, U.S. Pat. No. 10,774,141, or International Patent Publication No. WO2020/096381, the contents of each of which are incorporated by reference herein.

In some embodiments, an anti-IL-4Rα antibody or antigen-binding fragment thereof for use in the methods of the present disclosure comprises one or more CDR, HCVR, and/or LCVR sequences set forth in Table 7 below.

In some embodiments, an anti-IL-4Rα antibody comprises (i) an HCVR comprising the amino acid sequence of SEQ ID NO:32 (SCB-VH-59), SEQ ID NO:33 (SCB-VH-60), SEQ ID NO:34 (SCB-VH-61), SEQ ID NO:35 (SCB-VH-62), SEQ ID NO:36 (SCB-VH-63), SEQ ID NO:37 (SCB-VH-64), SEQ ID NO:38 (SCB-VH-65), SEQ ID NO:39 (SCB-VH-66), SEQ ID NO:40 (SCB-VH-67), SEQ ID NO:41 (SCB-VH-68), SEQ ID NO:42 (SCB-VH-69), SEQ ID NO:43 (SCB-VH-70), SEQ ID NO:44 (SCB-VH-71), SEQ ID NO:45 (SCB-VH-72), SEQ ID NO:46 (SCB-VH-73), SEQ ID NO:47 (SCB-VH-74), SEQ ID NO:48 (SCB-VH-75), SEQ ID NO:49 (SCB-VH-76), SEQ ID NO:50 (SCB-VH-77), SEQ ID NO:51 (SCB-VH-78), SEQ ID NO:52 (SCB-VH-79), SEQ ID NO:53 (SCB-VH-80), SEQ ID NO:54 (SCB-VH-81), SEQ ID NO:55 (SCB-VH-82), SEQ ID NO:56 (SCB-VH-83), SEQ ID NO:57 (SCB-VH-84), SEQ ID NO:58 (SCB-VH-85), SEQ ID NO:59 (SCB-VH-86), SEQ ID NO:60 (SCB-VH-87), SEQ ID NO:61 (SCB-VH-88), SEQ ID NO:62 (SCB-VH-89), SEQ ID NO:63 (SCB-VH-90), SEQ ID NO:64 (SCB-VH-91), SEQ ID NO:65 (SCB-VH-92), or SEQ ID NO:66 (SCB-VH-93); and (ii) an LCVR comprising the amino acid sequence of SEQ ID NO:12 (SCB-VL-39), SEQ ID NO:13 (SCB-VL-40), SEQ ID NO:14 (SCB-VL-41), SEQ ID NO:15 (SCB-VL-42), SEQ ID NO:16 (SCB-VL-43), SEQ ID NO:17 (SCB-VL-44), SEQ ID NO:18 (SCB-VL-45), SEQ ID NO:19 (SCB-VL-46), SEQ ID NO:20 (SCB-VL-47), SEQ ID NO:21 (SCB-VL-48), SEQ ID NO:22 (SCB-VL-49), SEQ ID NO:23 (SCB-VL-50), SEQ ID NO:24 (SCB-VL-51), SEQ ID NO:25 (SCB-VL-52), SEQ ID NO:26 (SCB-VL-53), SEQ ID NO:27 (SCB-VL-54), SEQ ID NO:28 (SCB-VL-55), SEQ ID NO:29 (SCB-VL-56), SEQ ID NO:30 (SCB-VL-57), or SEQ ID NO:31 (SCB-VL-58). In some embodiments, the anti-IL-4Rα antibody comprises an HCVR comprising the amino acid sequence of SEQ ID NO:64 (SCB-VH-91) and an LCVR comprising the amino acid sequence of SEQ ID NO:17 (SCB-VL-44), SEQ ID NO:27 (SCB-VL-54), or SEQ ID NO:28 (SCB-VL-55).

In some embodiments, an anti-IL-4Rα antibody comprises an amino acid sequence pair selected from the group consisting of: SEQ ID NOs:67/68 (MEDI-1-VH/MEDI-1-VL); SEQ ID NOs:69/70 (MEDI-2-VH/MEDI-2-VL); SEQ ID NOs:71/72 (MEDI-3-VH/MEDI-3-VL); SEQ ID NOs:73/74 (MEDI-4-VH/MEDI-4-VL); SEQ ID NOs:75/76 (MEDI-5-VH/MEDI-5-VL); SEQ ID NOs:77/78 (MEDI-6-VH/MEDI-6/VL); SEQ ID NOs:79/80 (MEDI-7-VH/MEDI-7-VL); SEQ ID NOs:81/82 (MEDI-8-VH/MEDI-8-VL); SEQ ID NOs:83/84 (MEDI-9-VH/MEDI-9-VL); SEQ ID NOs:85/86 (MEDI-10-VH/MEDI-10-VL); SEQ ID NOs:87/88 (MEDI-11-VH/MEDI-11/VL); SEQ ID NOs:89/90 (MEDI-12-VH/MEDI-12-VL); SEQ ID NOs:91/92 (MEDI-13-VH/MEDI-13-VL); SEQ ID NOs:93/94 (MEDI-14-VH/MEDI-14-VL); SEQ ID NOs:95/96 (MEDI-15-VH/MEDI-15-VL); SEQ ID NOs:97/98 (MEDI-16-VH/MEDI-16/VL); SEQ ID NOs:99/100 (MEDI-17-VH/MEDI-17-VL); SEQ ID NOs:101/102 (MEDI-18-VH/MEDI-18-VL); SEQ ID NOs:103/104 (MEDI-19-VH/MEDI-19-VL); SEQ ID NOs:105/106 (MEDI-20-VH/MEDI-20-VL); SEQ ID NOs:107/108 (MEDI-21-VH/MEDI-21-VL); SEQ ID NOs:109/110 (MEDI-22-VH/MEDI-22-VL); SEQ ID NOs:111/112 (MEDI-23-VH/MEDI-23-VL); SEQ ID NOs:113/114 (MEDI-24-VH/MEDI-24-VL); SEQ ID NOs:115/116 (MEDI-25-VH/MEDI-25-VL); SEQ ID NOs:117/118 (MEDI-26-VH/MEDI-26-VL); SEQ ID NOs:119/120 (MEDI-27-VH/MEDI-27-VL); SEQ ID NOs:121/122 (MEDI-28-VH/MEDI-28-VL); SEQ ID NOs:123/124 (MEDI-29-VH/MEDI-29-VL); SEQ ID NOs:125/126 (MEDI-30-VH/MEDI-30-VL); SEQ ID NOs:127/128 (MEDI-31-VH/MEDI-31-VL); SEQ ID NOs:129/130 (MEDI-32-VH/MEDI-32-VL); SEQ ID NOs:131/132 (MEDI-33-VH/MEDI-33-VL); SEQ ID NOs:133/134 (MEDI-34-VH/MEDI-34-VL); SEQ ID NOs:135/136 (MEDI-35-VH/MEDI-35-VL); SEQ ID NOs:137/138 (MEDI-36-VH/MEDI-36-VL); SEQ ID NOs:139/140 (MEDI-37-VH/MEDI-37-VL); SEQ ID NOs:141/142 (MEDI-38-VH/MEDI-38-VL); SEQ ID NOs:143/144 (MEDI-39-VH/MEDI-39-VL); SEQ ID NOs:145/146 (MEDI-40-VH/MEDI-40-VL); SEQ ID NOs:147/148 (MEDI-41-VH/MEDI-41-VL); SEQ ID NOs:149/150 (MEDI-42-VH/MEDI-42-VL); and SEQ ID NOs:151/152 (MEDI-37GL-VH/MEDI-37GL-VL).

In some embodiments, an anti-IL-4Rα antibody comprises (i) an HCVR comprising the amino acid sequence of SEQ ID NO:153 (AJOU-1-VH), SEQ ID NO:154 (AJOU-2-VH), SEQ ID NO:155 (AJOU-3-VH), SEQ ID NO:156 (AJOU-4-VH), SEQ ID NO:157 (AJOU-5-VH), SEQ ID NO:158 (AJOU-6-VH), SEQ ID NO:159 (AJOU-7-VH), SEQ ID NO:160 (AJOU-8-VH), SEQ ID NO:161 (AJOU-9-VH), SEQ ID NO:162 (AJOU-10-VH), SEQ ID NO:163 (AJOU-69-VH), SEQ ID NO:164 (AJOU-70-VH), SEQ ID NO:165 (AJOU-71-VH), SEQ ID NO:166 (AJOU-72-VH), or SEQ ID NO:167 (AJOU-83-VH); and (ii) an LCVR comprising the amino acid sequence of SEQ ID NO:168 (AJOU-33-V_{L}), SEQ ID NO:169 (AJOU-34-V_{L}), SEQ ID NO:170 (AJOU-35-V_{L}), SEQ ID NO:171 (AJOU-36-V_{L}), SEQ ID NO:172 (AJOU-37-V_{L}), SEQ ID NO:173 (AJOU-38-V_{L}), SEQ ID NO:174 (AJOU-39-V_{L}), SEQ ID NO:175 (AJOU-40-V_{L}), SEQ ID NO:176 (AJOU-41-V_{L}), SEQ ID NO:177 (AJOU-42-V_{L}), SEQ ID NO:178 (AJOU-77-V_{L}), SEQ ID NO:179 (AJOU-78-V_{L}), SEQ ID NO:180 (AJOU-79-V_{L}), SEQ ID NO:181 (AJOU-80-V_{L}), SEQ ID NO:182 (AJOU-86-V_{L}), SEQ ID NO:183 (AJOU-87-V_{L}), SEQ ID NO:184 (AJOU-88-V_{L}), SEQ ID NO:185 (AJOU-89-V_{L}), SEQ ID NO:186 (AJOU-90-V_{L}), or SEQ ID NO:187 (AJOU-91-V_{L}).

In some embodiments, an anti-IL-4Rα antibody comprises (i) an HCVR comprising the amino acid sequence of SEQ ID NO:188 (REGN-VH-3), SEQ ID NO:189 (REGN-VH-19), SEQ ID NO:190 (REGN-VH-35), SEQ ID NO:191 (REGN-VH-51), SEQ ID NO:192 (REGN-VH-67), SEQ ID NO:193 (REGN-VH-83), SEQ ID NO:194 (REGN-VH-99), SEQ ID NO:195 (REGN-VH-115), SEQ ID NO:196 (REGN-VH-147), or SEQ ID NO:197 (REGN-VH-163); and (ii) an LCVR comprising the amino acid sequence of SEQ ID NO:198 (REGN-VL-11), SEQ ID NO:199 (REGN-VL-27), SEQ ID NO:200 (REGN-VL-43), SEQ ID NO:201 (REGN-VL-59), SEQ ID NO:202 (REGN-VL-75), SEQ ID NO:203 (REGN-VL-91), SEQ ID NO:204 (REGN-VL-107), SEQ ID NO:205 (REGN-VL-123), SEQ ID NO:206 (REGN-VL-155), or SEQ ID NO:207 (REGN-VL-171).

In some embodiments, an anti-IL-4Rα antibody used in the methods of the present disclosure can have pH-dependent binding characteristics. For example, an anti-IL-4Rα antibody for use as disclosed herein may exhibit reduced binding to IL-4Rα at acidic pH as compared to neutral pH. Alternatively, an anti-IL-4Rα antibody for use as disclosed herein may exhibit enhanced binding to its antigen at acidic pH as compared to neutral pH. The expression “acidic pH” includes pH values less than about 6.2, e.g., about 6.0, 5.95, 5.9, 5.85, 5.8, 5.75, 5.7, 5.65, 5.6, 5.55, 5.5, 5.45, 5.4, 5.35, 5.3, 5.25, 5.2, 5.15, 5.1, 5.05, 5.0, or less. As used herein, the expression “neutral pH” means a pH of about 7.0 to about 7.4. The expression “neutral pH” includes pH values of about 7.0, 7.05, 7.1, 7.15, 7.2, 7.25, 7.3, 7.35, and 7.4.

In certain instances, “reduced binding to IL-4Rα at acidic pH as compared to neutral pH” is expressed in terms of a ratio of the K_{D} value of the antibody binding to IL-4Rα at acidic pH to the K_{D} value of the antibody binding to IL-4Rα at neutral pH (or vice versa). For example, an antibody or antigen-binding fragment thereof may be regarded as exhibiting “reduced binding to IL-4Rα at acidic pH as compared to neutral pH” for purposes of the present disclosure if the antibody or antigen-binding fragment thereof exhibits an acidic/neutral K_{D} ratio of about 3.0 or greater. In certain exemplary embodiments, the acidic/neutral K_{D} ratio for an antibody or antigen-binding fragment of the present disclosure can be about 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 20.0, 25.0, 30.0, 40.0, 50.0, 60.0, 70.0, 100.0, or greater.

Antibodies with pH-dependent binding characteristics may be obtained, e.g., by screening a population of antibodies for reduced (or enhanced) binding to a particular antigen at acidic pH as compared to neutral pH. Additionally, modifications of the antigen-binding domain at the amino acid level may yield antibodies with pH-dependent characteristics. For example, by substituting one or more amino acids of an antigen-binding domain (e.g., within a CDR) with a histidine residue, an antibody with reduced antigen-binding at acidic pH relative to neutral pH may be obtained.

### Preparation of Human Antibodies

Methods for generating human antibodies in transgenic mice are known in the art. Any such known methods can be used in the context of the present disclosure to make human antibodies that specifically bind to human IL-4R.

Using VELOCIMMUNE™ technology (see, for example, U.S. Pat. No. 6,596,541, Regeneron Pharmaceuticals) or any other known method for generating monoclonal antibodies, high affinity chimeric antibodies to IL-4R are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc., using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds IL-4R and that can be used in the methods disclosed herein comprises the three heavy chain CDRs (HCDR1, HCDR2 and HCDR3) contained within a heavy chain variable region (HCVR) having an amino acid sequence of SEQ ID NO: 1, and the three light chain CDRs (LCVR1, LCVR2, LCVR3) contained within a light chain variable region (LCVR) having an amino acid sequence of SEQ ID NO: 2. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. Biol. 273:927-948 (1997); and Martin et al., Proc. Natl. Acad. Sci. USA 86:9268-9272 (1989). Public databases are also available for identifying CDR sequences within an antibody.

### Pharmaceutical Compositions

In one aspect, the present disclosure provides methods that comprise administering an IL-4R antagonist to a subject, wherein the IL-4R antagonist (e.g., an anti-IL-4R antibody) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of antibody administered to a subject according to the methods of the present disclosure may vary depending upon the age and the size of the subject, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-IL-4R antibodies may be determined empirically; for example, subject progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351). Specific exemplary doses of anti-IL4R antibodies, and administration regimens involving the same, that can be used in the context of the present disclosure are disclosed elsewhere herein.

In some embodiments, an IL-4R antagonist or a pharmaceutical composition of the present disclosure is contained within a container. Thus, in another aspect, containers comprising an IL-4R antagonist or a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. Typically, a reusable pen delivery device utilizes a replaceable cartridge that contains a pharmaceutical composition. Once the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (sanofi-aventis, Frankfurt, Germany). Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park Ill.).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533. Other delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432).

In some embodiments, pharmaceutical compositions for use as described herein are prepared into dosage forms in a unit dose suited to fit a dose of the active ingredients. Such dosage forms in a unit dose include, for example, tablets, pills, capsules, injections (ampoules), suppositories, etc.

Exemplary pharmaceutical compositions comprising an anti-IL-4R antibody that can be used in the context of the present disclosure are disclosed, e.g., in U.S. Pat. No. 8,945,559.

### Dosage and Administration

In some embodiments, an IL-4R antagonist (e.g., anti-IL-4R antibody) is administered to a subject (e.g., a subject ≥6 months and <6 years of age) according to the methods of the present disclosure in a therapeutically effective amount. As used herein with reference to an IL-4R antagonist, the phrase “therapeutically effective amount” means an amount of IL-4R antagonist that results in one or more of: (a) an improvement in one or more AD-associated parameters (as mentioned elsewhere herein); and/or (b) a detectable improvement in one or more symptoms or indicia of atopic dermatitis.

In the case of an anti-IL-4R antibody, a therapeutically effective amount can be from about 0.05 mg to about 600 mg, e.g., about 0.05 mg, about 0.1 mg, about 1.0 mg, about 1.5 mg, about 2.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-IL-4R antibody. In some embodiments, a therapeutically effective amount is from about 50 mg to about 600 mg, or from about 100 mg to about 600 mg, or from about 200 mg to about 600 mg. In certain embodiments, 50 mg, 75 mg, 100 mg, 150 mg, 200 mg, or 300 mg of an anti-IL-4R antibody is administered to a subject.

The amount of IL-4R antagonist (e.g., anti-IL-4R antibody) contained within the individual doses may be expressed in terms of milligrams of antibody per kilogram of subject body weight (i.e., mg/kg). For example, the IL-4R antagonist may be administered to a subject at a dose of about 0.0001 to about 10 mg/kg of subject body weight, e.g., at a dose of about 1 mg/kg to about 10 mg/kg, at a dose of about 2 mg/kg to about 9 mg/kg, or at a dose of about 3 mg/kg to about 8 mg/kg. In some embodiments, the IL-4R antagonist may be administered to a subject at a dose of about 1 mg/kg, 2 mg/kg, 3 mg/kg, 4 mg/kg, 5 mg/kg, 6 mg/kg, 7 mg/kg, 8 mg/kg, 9 mg/kg, or 10 mg/kg. In some embodiments, the IL-4R antagonist is administered to a subject at a dose of about 3 mg/kg. In some embodiments, the IL-4R antagonist is administered to a subject at a dose of about 6 mg/kg. In some embodiments, the IL-4R antagonist is administered to a subject at a dose that is from 5 mg/kg to 10 mg/kg. In some embodiments, the IL-4R antagonist is administered to a subject at a dose that is at least about 5 mg/kg, e.g., at least 6 mg/kg.

In some embodiments, the IL-4R antagonist (e.g., anti-IL-4R antibody) is administered to the subject (e.g., subcutaneously) in an amount that results in a maximum serum concentration of the IL-4R antagonist (i.e., Cₘₐₓ) in the subject of at least 20 mg/L, e.g., at least 25 mg/L, 30 mg/L, at least 35 mg/L, at least 40 mg/L, or at least 45 mg/L. In some embodiments, the IL-4R antagonist (e.g., anti-IL-4R antibody) is administered to the subject (e.g., subcutaneously) in an amount that results in a total exposure to the IL-4R antagonist (i.e., AUC) in the subject of at least 120 day·mg/L, e.g., at least 125 day·mg/L, at least 130 day·mg/L, at least 150 day·mg/L, at least 200 day·mg/L, at least 250 day·mg/L, at least 300 day·mg/L, at least 350 day·mg/L, at least 400 day·mg/L, at least 450 day·mg/L, at least 500 day·mg/L, at least 550 day·mg/L, at least 600 day·mg/L, or at least 650 day·mg/L.

In some embodiments, the methods disclosed herein comprise administering an IL-4R antagonist to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved.

In some embodiments, multiple doses of the IL-4R antagonist are administered (e.g., subcutaneously) to a subject at a dosing frequency that results in the subject maintaining a serum concentration of the IL-4R antagonist over a defined period of time (e.g., over a period of at least 4 weeks, or over a period of at least 1, 2, 3, 4, 5, 6, 7, 8, 9, 10, 11, 12 months or longer) of at least 25 mg/L, e.g., at least 30 mg/L, at least 35 mg/L, at least 40 mg/L, or at least 45 mg/L. In some embodiments, multiple doses of the IL-4R antagonist are administered (e.g., subcutaneously) to a subject at a dosing frequency that results in the subject maintaining a total exposure to the IL-4R antagonist of at least 130 day·mg/L (e.g., at least 150 day·mg/L, at least 200 day·mg/L, at least 250 day·mg/L, at least 300 day·mg/L, at least 350 day·mg/L, at least 400 day·mg/L, at least 450 day·mg/L, at least 500 day·mg/L, at least 550 day·mg/L, at least 600 day·mg/L, or at least 650 day·mg/L) for at least one week, at least two weeks, at least three weeks, at least four weeks, or longer (e.g., for at least 1, 2, 3, 4, 5, 6, 7, 8, 9, 10, 11, or 12 months or longer).

In some embodiments, multiple doses of an IL-4R antagonist are administered to a subject over a defined time course. In some embodiments, the methods of the present disclosure comprise sequentially administering to a subject multiple doses of an IL-4R antagonist. As used herein, “sequentially administering” means that each dose of IL-4R antagonist is administered to the subject at a different point in time, e.g., on different days separated by a predetermined interval (e.g., hours, days, weeks or months). In some embodiments, the methods of the disclosure comprise sequentially administering to the patient a single initial dose of an IL-4R antagonist, followed by one or more secondary doses of the IL-4R antagonist, and optionally followed by one or more tertiary doses of the IL-4R antagonist.

The terms “initial dose,” “secondary doses,” and “tertiary doses,” refer to the temporal sequence of administration of the IL-4R antagonist. Thus, the “initial dose” is the dose which is administered at the beginning of the treatment regimen (also referred to as the “loading dose”); the “secondary doses” are the doses which are administered after the initial dose; and the “tertiary doses” are the doses which are administered after the secondary doses. The initial, secondary, and tertiary doses may all contain the same amount of IL-4R antagonist, but generally may differ from one another in terms of frequency of administration. In certain embodiments, however, the amount of IL-4R antagonist contained in the initial, secondary and/or tertiary doses varies from one another (e.g., adjusted up or down as appropriate) during the course of treatment. In certain embodiments, one or more (e.g., 1, 2, 3, 4, or 5) doses are administered at the beginning of the treatment regimen as “loading doses” followed by subsequent doses that are administered on a less frequent basis (e.g., “maintenance doses”). In some embodiments, the initial dose and the one or more secondary doses each contain the same amount of the IL-4R antagonist. In other embodiments, the initial dose comprises a first amount of the IL-4R antagonist, and the one or more secondary doses each comprise a second amount of the IL-4R antagonist. For example, the first amount of the IL-4R antagonist can be 1.5×, 2×, 2.5×, 3×, 3.5×, 4× or 5× or more than the second amount of the IL-4R antagonist. In some embodiments, one or more maintenance doses of the IL-4R antagonist are administered without a loading dose.

In some embodiments, a loading dose is a “split dose” that is administered as two or more doses (e.g., 2, 3, 4, or 5 doses) that are administered on separate days. In some embodiments, a loading dose is administered as a split dose wherein the two or more doses are administered at least about one week apart. In some embodiments, a loading dose is administered as a split dose wherein the two or more doses are administered about 1 week, 2 weeks, 3 weeks, or 4 weeks apart. In some embodiments, the loading dose is split evenly over the two or more doses (e.g., half of the loading dose is administered as the first portion and half of the loading dose is administered as the second portion). In some embodiments, the loading dose is split unevenly over the two or more doses (e.g., more than half of the loading dose is administered as the first portion and less than half of the loading dose is administered as the second portion).

In some embodiments, each secondary and/or tertiary dose is administered 1 to 14 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, or more) weeks after the immediately preceding dose. The phrase “the immediately preceding dose,” as used herein, means, in a sequence of multiple administrations, the dose of IL-4R antagonist which is administered to a patient prior to the administration of the very next dose in the sequence with no intervening doses.

The methods of the disclosure may comprise administering to a patient any number of secondary and/or tertiary doses of an IL-4R antagonist. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments involving multiple secondary doses, each secondary dose is administered at the same frequency as the other secondary doses. For example, each secondary dose may be administered to the patient 1 to 2 weeks after the immediately preceding dose. Similarly, in some embodiments involving multiple tertiary doses, each tertiary dose is administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### Combination Therapies

In some embodiments, the methods of the present disclosure comprise administering to the subject (e.g., a subject ≥6 months and <6 years of age) an IL-4R antagonist according to the disclosure (e.g., an anti-IL-4R antibody) in combination with one or more additional therapeutic agents. In some embodiments, the additional therapeutic agent is a topical therapeutic agent, e.g., a TCS or a topical nonsteroidal medication such as a TCI or crisaborole. As used herein, the expression “in combination with” means that the topical therapy (e.g., TCS) is administered before, after, or concurrent with the IL-4R inhibitor. The term “in combination with” also includes sequential or concomitant administration of IL-4R inhibitor and the topical therapy (e.g., TCS).

For example, when administered “before” the pharmaceutical composition comprising the IL-4R antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the IL-4R antagonist. When administered “after” the pharmaceutical composition comprising the IL-4R antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the IL-4R antagonist. Administration “concurrent” or with the pharmaceutical composition comprising the IL-4R antagonist means that the additional therapeutic agent is administered to the subject in a separate dosage form within less than 5 minutes (before, after, or at the same time) of administration of the pharmaceutical composition comprising the IL-4R antagonist, or administered to the subject as a single combined dosage formulation comprising both the additional therapeutic agent and the IL-4R antagonist.

In some embodiments, the additional therapeutic agent is a TCS. In some embodiments, the TCS is a medium-potency TCS. In some embodiments, the TCS is a low-potency TCS. In some embodiments, the additional therapeutic agent is a TCI. In some embodiments, the additional therapeutic agent is crisaborole.

### EXAMPLES

The following examples are put forth so as to provide those of ordinary skill in the art with a complete disclosure and description of how to make and use the methods and compositions of the disclosure, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Clinical Trial Investigating the Pharmacokinetics, Efficacy, and Safety of Dupilumab in Children Aged 6 Months to <6 Years with Severe Uncontrolled Atopic Dermatitis

### Study Design and Objectives

This was an open-label, multicenter, phase 2, sequential, two-age cohort, two dose level study (LIBERTY AD PRE-SCHOOL; NCT03346434) investigating the PK, safety and efficacy of subcutaneous dupilumab. Dupilumab is a fully human anti-IL-4R antibody comprising a heavy chain comprising the amino acid sequence of SEQ ID NO:9 and a light chain comprising the amino acid sequence of SEQ ID NO:10; an HCVR/LCVR amino acid sequence pair comprising SEQ ID NOs:1/2; and heavy and light chain CDR sequences comprising SEQ ID NOs:3-8. Older patients (aged ≥2 to <6 years) were enrolled first, followed by the younger cohort (aged ≥6 months to <2 years). A subgroup of 10 patients in each cohort was treated with the lower weight-based dose (3 mg/kg) followed by another subgroup treated with the higher dose (6 mg/kg). To ensure adequate distribution of patients within each age cohort, the maximum number of patients enrolled at a given dose level was restricted to 7 patients in each of the subgroups: 2 to <4 years and 4 to <6 years in the older cohort, and 6 months to <1 year and 1 to <2 years in the younger cohort.

The study consisted of a screening period (Day −35 to Day −1), baseline visit (Day 1) and single-dose treatment on Day 1, followed by a 4-week PK sampling period. Patients were then offered the opportunity to enroll in an open-label extension (OLE) study R668-AD-1434 (LIBERTY AD PED-OLE, NCT02612454). Those who declined or were ineligible to participate in the OLE were followed for an additional 4 weeks.

This study was conducted in accordance with the provisions of the Declaration of Helsinki, the International Conference on Harmonization Good Clinical Practices guideline, and applicable regulatory requirements. The protocol was reviewed and approved by institutional review boards/ethics committees at all sites. For all patients, written informed consent was obtained from a parent or legal guardian.

### Patient Population

This study enrolled pediatric patients (male or female aged ≥6 months to <6 years at the time of screening) having severe AD with a documented recent history of inadequate response to topical AD medication(s).

Inclusion Criteria: A patient had to meet the following criteria to be eligible for inclusion in the study: (1) male or female, ≥6 months to <6 years of age at time of screening visit; (2) diagnosis of AD according to the American Academy of Dermatology consensus criteria (Eichenfield 2003) at screening visit; (3) documented recent history (within 6 months before the screening visit) of inadequate response to topical AD medication(s); (4) IGA=4 at screening and baseline visits; (5) EASI≥21 at the screening and baseline visits; (6) BSA≥15% at screening and baseline visits; (7) have applied a stable dose of topical emollient (moisturizer) twice daily for at least the 7 consecutive days immediately before the baseline visit (for part B of the study only); (8) parent or legal guardians, as appropriate, are able to understand and complete the study requirements and study-related questionnaires.

NOTE: Patients who are unable to achieve and/or maintain remission and low disease activity (an IGA score of less than 3) despite treatment with a daily regimen of medium to higher potency TCS (±TCI as appropriate), applied for ≥28 days of use, or for the maximum duration recommended by the product prescribing information, whichever is shorter, will meet the definition of inadequate response for the purpose of this study. Patients with documented systemic treatment for AD in the past 6 months are also considered as inadequate responders to topical treatments and are potentially eligible for treatment with dupilumab after appropriate washout. Acceptable documentation includes contemporaneous chart notes that record topical medication prescription and treatment outcome, or investigator documentation based on communication with the patient's treating physician. If documentation is inadequate, potential patients may be offered a course of treatment with a daily regimen of TCS of medium or higher potency (±TCI as appropriate), applied for at least 28 days during the screening period, or for the maximum duration recommended by the product prescribing information, whichever is shorter. Patients who demonstrate inadequate response during this period, as defined above, will still be eligible for inclusion in the study.

Exclusion Criteria: The following were exclusion criteria for the study: (1) Participation in a prior dupilumab clinical study; (2) History of important side effects of medium potency topical corticosteroids (e.g., intolerance to treatment, hypersensitivity reactions, significant skin atrophy, systemic effects), as assessed by the investigator or patient's treating physician; (3) ≥30% of the total lesional surface located on areas of thin skin that cannot be safely treated with medium-potency TCS (e.g. face, neck, intertriginous areas, genital areas, areas of skin atrophy) at baseline (only applicable for part B of the study); (4) Treatment with an investigational drug at any time before the baseline visit; (5) Treatment with a TCI within 2 weeks prior to the baseline visit (only applicable for part B of the study); (6) Having used any of the following treatments within 4 weeks before the baseline visit, or within a period equal to 5 times the half-life of the drug, before the baseline visit, whichever is longer: (a) immunosuppressive/immunomodulating drugs (e.g., systemic corticosteroids, cyclosporine, mycophenolate-mofetil, interferon gamma, Janus kinase inhibitors, azathioprine, methotrexate, etc.); (b) phototherapy for AD; (7) Treatment with biologics, as follows: (a) any cell-depleting agents including but not limited to rituximab: within 6 months before the baseline visit, or until lymphocyte and CD 19+ lymphocyte count returns to normal, whichever is longer; (b) other biologics: within 5 half-lives (if known) or 16 weeks before the baseline visit, whichever is longer; (8) Treatment with crisaborole within 2 weeks prior to the baseline visit; (9) Treatment with a live (attenuated) vaccine within 4 weeks before the baseline visit. [Note: For patients who have vaccination with live, attenuated vaccines planned during the course of the study (based on national vaccination schedule/local guidelines), it will be determined, after consultation with a pediatrician, whether the administration of vaccine can be postponed until after the end of study, or preponed to before the start of the study, without compromising the health of the patient: (a) Patients for whom administration of live (attenuated) vaccine can be safely postponed would be eligible to enroll into the study. (b) Patients who have their vaccination preponed can enroll in the study only after a gap of 4 weeks following administration of the vaccine.] (10) Planned or anticipated use of any prohibited medications and procedures during study treatment; (11) Initiation of treatment of AD with prescription moisturizers or moisturizers containing additives such as ceramide, hyaluronic acid, urea, or filaggrin degradation products during the screening period (patients may continue using stable doses of such moisturizers if initiated before the screening visit) (for part B of the study only); (12) Active chronic or acute infection requiring treatment with systemic antibiotics, antivirals, antiprotozoals, or antifungals within 2 weeks before the baseline visit. [Note: patients may be rescreened after infection resolves. A patient with mild, localized superficial infection can be included in the study based on investigator discretion.]; (13) Established diagnosis of a primary immunodeficiency disorder (e.g., severe combined immunodeficiency, Wiskott Aldrich Syndrome, DiGeorge Syndrome, X-linked Agammaglobulinemia, common variable immunodeficiency), or secondary immunodeficiency. Patients suspected to have immunodeficiency based on their clinical presentation (history of invasive opportunistic infections e.g. tuberculosis, histoplasmosis, listeriosis, coccidioidomycosis, pneumocystosis, chronic mucocutaneous candidiasis etc. or otherwise recurrent infections of abnormal frequency or prolonged duration suggesting an immune compromised status, as judged by the investigator) will also be excluded from the study; (14) Eczema as part of a genodermatosis syndrome like Netherton's syndrome, Hyper IgE syndrome, Wiskott-Aldrich Syndrome, etc.; (15) Known history of human immunodeficiency virus (HIV) infection or HIV seropositivity at the screening visit; (16) Established diagnosis of hepatitis B viral infection at the time of screening or is positive for hepatitis B surface antigen (HBsAg) or hepatitis B core antibody (HBcAb) at the time of screening. [Note: Patients who are HBsAg negative and HBsAb positive are considered immune after a natural infection has cleared or they have been vaccinated against hepatitis B. Therefore, they are acceptable for the study. These patients will be allowed to enroll into the study, but will be followed using routine clinical and liver function tests.]; (17) Established diagnosis of hepatitis C viral infection at the time of screening or is positive for hepatitis C antibody at the screening visit; (18) History past or current tuberculosis or other mycobacterial infection; (19) Have known hepatic disease or are on current treatment for hepatic disease including but not limited to acute or chronic hepatitis, cirrhosis, or hepatic failure, or has evidence of liver disease as indicated by persistent (confirmed by repeated tests ≥2 weeks apart) elevated transaminases (alanine aminotransferase [ALT] and/or aspartate aminotransferase [AST]) >3 times the upper limit of normal (ULN) during the screening period; (20) Presence of any one or more of the following abnormalities in laboratory test results at screening: (i) Platelets ≤100×10³/μL; (ii) Neutrophils ≤1.0×10³/μL for patients <1 year of age; neutrophils ≤1.5×10³/μL for patients 1 year to <6 years of age; (iii) Eosinophils >5000/μL; (iv) Creatine phosphokinase (CPK) >5×ULN; (v) Serum creatinine >1.5×ULN. [Note: If an abnormal value is detected at screening, a repeat test should be performed to confirm the abnormality. If the repeat test confirms the abnormality, the patient will be categorized as a screen failure.]; (21) Presence of skin comorbidities that may interfere with study assessments, including but not limited to conditions like scabies, seborrheic dermatitis, cutaneous T cell lymphoma, psoriasis, etc.; (22) History of malignancy before the baseline visit; (23) Diagnosed active endoparasitic infections; suspected or high risk of endoparasitic infection, unless clinical and (if necessary) laboratory assessment have ruled out active infection before randomization; (24) Severe concomitant illness(es) that, in the investigator's judgment, would adversely affect the patient's participation in the study. Examples include, but are not limited to patients with short life expectancy, patients with major congenital malformations, patients with cardiovascular conditions (e.g. major, clinically significant congenital cardiovascular abnormalities), severe renal conditions, hepato-biliary conditions (e.g. Child-Pugh class B or C), active major autoimmune diseases (e.g. lupus, inflammatory bowel disease etc.), other severe endocrinological, gastrointestinal, metabolic, pulmonary, neurological or lymphatic diseases. The specific justification for patients excluded under this criterion will be noted in study documents (chart notes, case report forms [CRF], etc.). (25) Any other medical or psychological condition including relevant laboratory abnormalities at screening that, in the opinion of the investigator, suggest a new and/or insufficiently understood disease, may present an unreasonable risk to the study patient as a result of his/her participation in this clinical trial, may make patient's participation unreliable, or may interfere with study assessments; (26) Planned major surgical procedure during the patient's participation in this study; (27) Patient or his/her immediate family is a member of the dupilumab investigational team.

### Study Treatments

The 6 mg/kg dose was anticipated to provide drug exposure comparable to a single dose of dupilumab 300 mg in adult patients. The 3 mg/kg dose was evaluated first within each age group, to allow safety evaluation before progressing to the 6 mg/kg dose. The results from this phase 2 study were planned to inform dose selection for the pivotal, randomized, double-blinded, parallel-group, placebo-controlled, phase 3 study (LIBERTY AD INFANT) to evaluate the efficacy, safety, and immunogenicity of multiple doses of dupilumab administered concomitantly with TCS over 16 weeks. Standardized, low-to-medium potency TCS with or without TCI were allowed; high-potency TCS, systemic non-steroidal immunosuppressants, and systemic corticosteroids could be used only as rescue treatment. The use of crisaborole was also permitted, with the exception of the 2-week period leading up to the baseline visit, and consistent with local country guidelines and product prescribing information. Use of prescription moisturizers and moisturizers containing additives such as ceramide, hyaluronic acid, urea, or filaggrin degradation products were allowed as long as the use of such moisturizers had already been initiated before the screening visit. Initiation of treatment of AD with such moisturizers during the study was not allowed. Medications used to treat chronic disease such as diabetes, hypertension, and asthma were also permitted.

### Outcomes Assessed

Primary endpoints were: the concentration of functional dupilumab in serum over time and PK parameters (summary statistics of drug concentration and PK parameters); incidence and severity of treatment-emergent adverse events (TEAEs) throughout the study.

Secondary endpoints were: incidence of serious adverse events (SAEs) and severe TEAEs up to Week 4; percentage change in EASI (scale of 0-72) and SCORing Atopic Dermatitis (SCORAD) score (scale 0-103) from baseline to Week 4; proportion of patients with an IGA score of 0 or 1 (on a 5-point scale) at Week 4.

Other endpoints were: proportions of patients with ≥75% improvement from baseline in EASI (EASI-75) or ≥50% improvement from baseline in EASI (EASI-50) at Week 4; percentage change in caregiver-reported Peak Pruritus numerical rating scale (NRS) (scale 0-10) from baseline to Week 4; change in BSA affected from baseline to Week 4. Per protocol, the study visit for Week 3 was defined as Day 18±3 days, and Week 4 as Day 29±3 days.

Procedures for assessing efficacy (e.g., using EASI, SCORAD, IGA, BSA, NRS, or other methods of assessment) are described below and are also described in WO 2018/057776, incorporated by reference herein.

Investigator's Global Assessment: The IGA is an assessment instrument used in clinical studies to rate the severity of AD globally, based on a 5-point scale ranging from 0 (clear) to 4 (severe). The IGA score can be assessed at screening, baseline and on specified days during and/or after treatment.

Eczema Area and Severity Index: The EASI is a validated measure used in clinical practice and clinical trials to assess the severity and extent of AD (Hanifin et al 2001, Exp. Dermatol. 10: 11-18). The EASI is a composite index with scores ranging from 0 to 72. Four AD disease characteristics (erythema, thickness [induration, papulation, edema], scratching [excoriation], and lichenification) each are assessed for severity by the investigator or designee on a scale of “0” (absent) through “3” (severe). In addition, the area of AD involvement is assessed as a percentage by body area of head, trunk, upper limbs, and lower limbs, and converted to a score of 0 to 6. In each body region, the area is expressed as 0, 1 (1% to 9%), 2 (10% to 29%), 3 (30% to 49%), 4 (50% to 69%), 5 (70% to 89%), or 6 (90% to 100%). The EASI score can be assessed at screening, baseline and on specified days during and/or after treatment.

SCORing Atopic Dermatitis: The SCORing Atopic Dermatitis (SCORAD) is a validated tool used in clinical research and clinical practice that was developed to standardize the evaluation of the extent and severity of AD (European Task Force on Atopic Dermatitis 1993, Dermatol. 186: 23-31). There are 3 components to the assessment: A=extent or affected BSA, B=severity, and C=subjective symptoms. The extent of AD is assessed as a percentage of each defined body area and reported as the sum of all areas, with a maximum score of 100% (assigned as “A” in the overall SCORAD calculation). The severity of 6 specific symptoms of AD (redness, swelling, oozing/crusting, excoriation, skin thickening/lichenification, and dryness) is assessed using the following scale: none (0), mild (1), moderate (2), or severe (3) (for a maximum of 18 total points, assigned as “B” in the overall SCORAD calculation). Subjective assessment of itch and sleeplessness is recorded for each symptom by the patient or relative on a Visual Analogue Scale, where 0 is no itch (or sleeplessness) and 10 is the worst imaginable itch (or sleeplessness), with a maximum possible score of 20. This parameter is assigned as “C” in the overall SCORAD calculation. The SCORAD is calculated as: A/5+7B/2+C where the maximum is 103. The SCORAD score can be assessed at screening, baseline and on specified days during and/or after treatment.

Body Surface Area Involvement of Atopic Dermatitis: Body surface area (BSA) affected by AD is assessed for each section of the body using the rule of nines (the possible highest score for each region is: head and neck [9%], anterior trunk [18%], back [18%], upper limbs [18%], lower limbs [36%], and genitals [1%]) and is reported as a percentage of all major body sections combined. BSA can be assessed at screening, baseline and on specified days during and/or after treatment.

Peak Pruritus Numeric Rating Scale: Peak Pruritus Numeric Rating Scale (NRS) is a validated patient-reported measure for evaluating worst itch intensity (Yosipovitch et al., Br J Dermatol, 2019, 181:761-769). This is an 11-point scale (0 to 10), in which 0 indicates no itching while 10 indicates worst itching possible, in which the patient (or caregiver) assesses the intensity of peak (worst) pruritus (itch) during the past 24 hours.

### Pharmacokinetic Analysis

Functional dupilumab concentrations in serum were analyzed using a validated enzyme-linked immunosorbent assay (ELISA) as previously described. The lower limit of quantitation (LLoQ) for dupilumab in undiluted human serum is 0.0780 mg/L. Serum for PK analyses was collected at baseline (before dupilumab injection) and on study days 3, 8, 18, and 29.

PK parameters, including maximum concentration (Cₘₐₓ), dose-normalized Cₘₐₓ (Cₘₐₓ/Dose), time to maximum concentration (tₘₐₓ), last observed concentration (Cₗₐₛₜ), time to last observed concentration area under the curve (AUC) from time zero to the last (tₗₐₛₜ), observed concentration (AUCₗₐₛₜ), and dose-normalized AUCₗₐₛₜ (AUCₗₐₛₜ/Dose), were determined using non-compartmental methods and actual sampling times. Mean concentration-time profiles are presented using nominal sampling times.

### Biomarker Analysis

Serum samples were assayed for the measurement of CCL17/TARC, using a validated commercial ELISA (human CCL17/TARC Quantikine ELISA Kit #SDN00, R&D Systems Inc., Minneapolis, Minn., USA) according to manufacturer's instructions, and for total IgE using the immunonephelometry methodology on the BN II instrument. Blood eosinophil counts were measured using the Coulter LH 750 Hematology Analyzer instrument, using the volume, conductivity and scatter (VCS) flow technology.

### Statistical Analysis

Because the primary objective was to evaluate safety and PK, no formal power calculations based on efficacy endpoints were performed. A total of 10 patients in each dose group was considered adequate to characterize the safety and PK profiles. Descriptive statistics of functional dupilumab serum concentration at each time point by dose were reported from the PK analysis set (all treated patients who received any study drug and who had ≥1 non-missing functional dupilumab measurement post-dose). Safety and efficacy were assessed in the safety analysis set consisting of all treated patients who received ≥1 dose of dupilumab. Efficacy analyses were performed using an observed method, without censoring. Because of the small cohorts, we report no inferential statistical analyses; all efficacy outcomes are summarized by descriptive statistics. All analyses were performed using SAS Version 9.4 (Cary, N.C., USA) or higher.

### Results

40 patients (20 aged ≥2 to <6 years and 20 aged ≥6 months to <2 years; 10 at each dose level within an age cohort) were screened and enrolled. Patient screening was done in 21 of 30 sites initiated in the USA, UK, and Germany. All patients in the older cohort completed the study and transitioned to the OLE study; in the younger cohort, 1 patient withdrew consent and was discontinued from study prematurely during the safety follow-up period, and 2 patients completed the study but did not continue into the OLE. All patients were included in the safety analysis set.

Of all study patients, 10 were aged ≥4 to <6 years, 10 were ≥2 to <4 years old, 14 were ≥1 to <2 years old, and 6 were ≥6 months to <1 year. Baseline demographics and characteristics were, in general, comparable between the treatment groups within each age cohort. Overall, disease characteristics were consistent with severe AD (Table 1). In the older cohort, 40% had used systemic AD medications including 25% who had used corticosteroids and 20% non-steroidal immunosuppressants. All patients had ≥1 concurrent atopic/allergic comorbidity at baseline; more than half had food allergy or allergic rhinitis. In the younger cohort, 40% of patients had previously used systemic medications for AD, including 35% who had used corticosteroids and 5% non-steroidal immunosuppressants (Table 1). Most patients had ≥1 concurrent atopic/allergic comorbidity at baseline, of which more than half had food allergy (Table 1).

TABLE 1
Baseline demographics and clinical characteristics

| | ≥2 to <6 years of age | | ≥6 months to <2 years of age | |
|---|---|---|---|---|
| | Dupilumab | Dupilumab | Dupilumab | Dupilumab |
| | 3 mg/kg | 6 mg/kg | 3 mg/kg | 6 mg/kg |
| | (n = 10) | (n = 10) | (n = 10) | (n = 10) |
| Age, mean (SD), months | 45.8 (16.13) | 53.2 (11.23) | 15.4 (6.96) | 15.9(5.51) |

Age category, n (%), months

| | | | | |
|---|---|---|---|---|
| ≥48 to <72 | 4 (40.0) | 6 (60.0) | N/A | N/A |
| ≥24 to <48 | 6 (60.0) | 4 (40.0) | N/A | N/A |
| ≥12 to <24 | N/A | N/A | 7 (70.0) | 3 (30.0) |
| ≥6 to <12 | N/A | N/A | 3 (30.0) | 7 (70.0) |
| Male, n (%) | 6 (60.0) | 7 (70.0) | 9 (90.0) | 8 (80.0) |

Race, n (%)

| | | | | |
|---|---|---|---|---|
| White | 7 (70.0) | 8 (80.0) | 6 (60.0) | 7 (70.0) |
| Black or African American | 3 (30.0) | 1 (10.0) | 1 (10.0) | 2 (20.0) |
| Asian | 0 | 1 (10.0) | 2 (20.0) | 1 (10.0) |
| Weight, mean (SD), kg | 16.57 (3.83) | 17.61 (3.59) | 9.66 (1.67) | 9.89 (1.96) |
| BMI, mean (SD), kg/m² | 16.15 (1.93) | 16.29 (1.72) | 17.11 (1.81) | 17.53 (2.59) |
| Duration of AD, mean (SD), | 40.9 (14.32) | 50.6 (10.73) | 13.7 (6.55) | 13.4 (5.44) |
| months | | | | |
| EASI, mean (SD), | 35.2 (9.21) | 40.2 (11.81) | 34.4 (14.25) | 36.1 (12.94) |
| scale 0-72 | | | | |
| Caregiver-reported Peak | 8.4 (1.24) | 8.1 (1.45) | 7.6 (2.55) | 8.5 (0.71) |
| Pruritus NRS, mean (SD), | | | | |
| scale 0-10 | | | | |
| BSA involvement, mean (SD), | 58.1 (11.09) | 67.5 (16.05) | 55.3 (25.66) | 57.9 (21.37) |
| % | | | | |
| SCORAD score, mean (SD), | 73.5 (10.20) | 75.1 (8.08) | 69.8 (13.10) | 75.9 (11.74) |
| range 0-103 | | | | |
| Prior systemic medication use | 5 (50.0) | 3 (30.0) | 3 (30.0) | 5 (50.0) |
| for AD, | | | | |
| n (%) | | | | |
| Oral corticosteroids | 4 (40.0) | 1 (10.0) | 2 (20.0) | 5 (50.0) |
| Non-steroidal | 1 (10.0) | 3 (30.0) | 1 (10.0) | 0 |
| immunosuppressants | | | | |
| Cyclosporine | 1 (10.0) | 1 (10.0) | 0 | 0 |
| Methotrexate | 0 | 2 (20.0) | 1 (10.0) | 0 |
| Proportions of patients with ≥1 | 10 (100.0) | 10 (100.0) | 6 (60.0) | 8 (80.0) |
| current history of | | | | |
| atopic/allergic diseases other | | | | |
| than AD,^{†} n (%) | | | | |
| Food allergy | 10 (100.0) | 8 (80.0) | 5 (50.0) | 7 (70.0) |
| Allergic rhinitis | 7 (70.0) | 6 (60.0) | 3 (30.0) | 3 (30.0) |
| Other allergies | 6 (60.0) | 4 (40.0) | 1 (10.0) | 5 (50.0) |
| Asthma | 3 (30.0) | 3 (30.0) | 0 | 0 |
| Hives | 3 (30.0) | 2 (20.0) | 1 (10.0) | 1 (10.0) |
| Allergic conjunctivitis | 0 | 2 (20.0) | 0 | 0 |
| (keratoconjunctivitis) | | | | |

^{†}Comorbidities were documented based on history provided by the caregiver.
AD, atopic dermatitis;
BMI, body mass index;
BSA, body surface area;
EASI, Eczema Area and Severity Index;
N/A, not applicable;
NRS, numerical rating scale;
SCORAD, SCORing Atopic Dermatitis;
SD, standard deviation.

### Dupilumab Pharmacokinetics

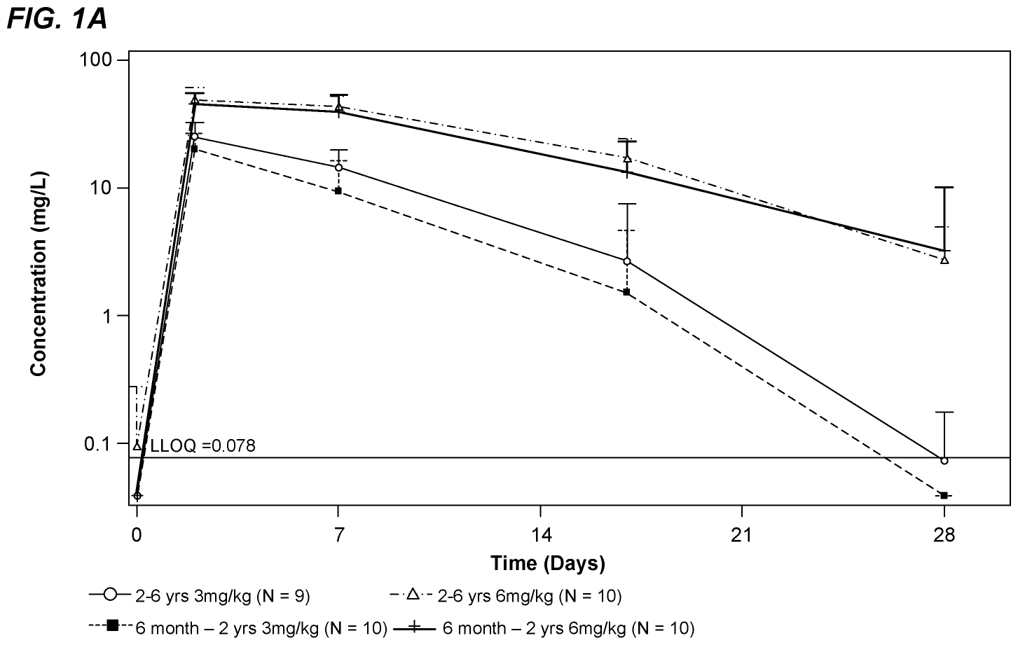

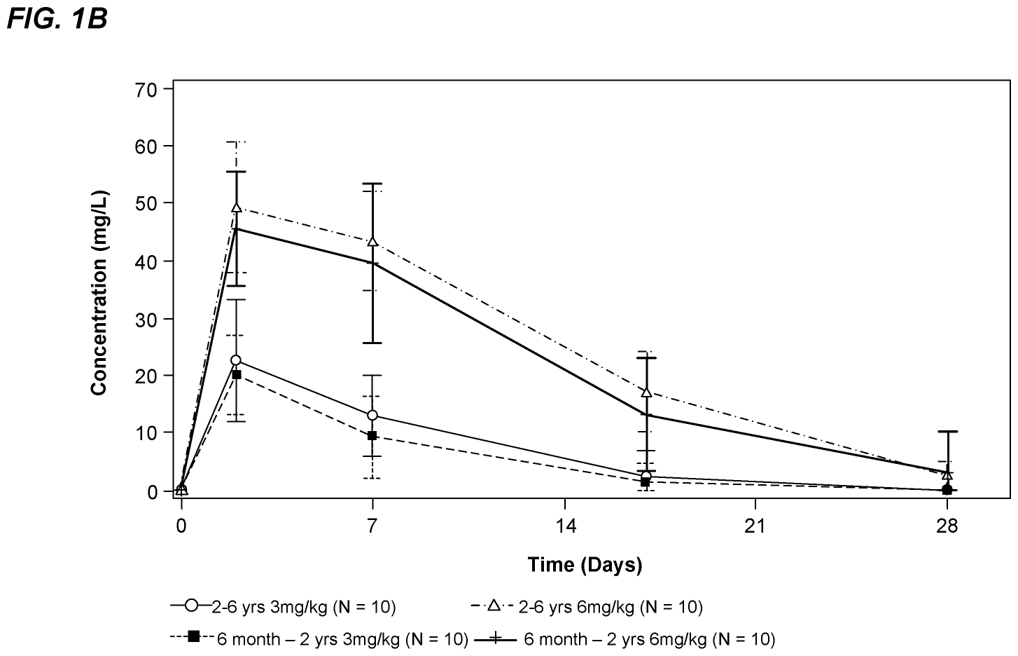

Within each age cohort, the higher 6 mg/kg dupilumab dose led to higher concentrations in serum that persisted for longer periods of time than the lower 3 mg/kg dose. Maximum concentrations of dupilumab in serum were similar between age cohorts at each dose level and were observed 2 days after injection in most patients (FIG. 1A). Mean Cₘₐₓ in the 3 mg/kg and 6 mg/kg dose groups of the older cohort were 25.2 mg/L and 49.8 mg/L, respectively, and in the younger cohort 20.1 mg/L and 46.1 mg/L, respectively (Table 2). Total exposure of dupilumab increased in a greater than dose-proportional manner between dose levels within each age cohort and was slightly higher in the older cohort at each dose level. Mean AUCₗₐₛₜ increased from 215 day·mg/L for the 3 mg/kg dose to 670 day·mg/L for the 6 mg/kg dose in the older cohort, and from 133 day·mg/L for the 3 mg/kg dose to 519 day·mg/L for the 6 mg/kg dose in the younger cohort (Table 2, FIG. 1B). Mean concentrations of dupilumab in serum were below the LLoQ by Week 4 in the 3 mg/kg dose groups, but remained measurable in the 6 mg/kg groups.

TABLE 2
Non-compartmental pharmacokinetic parameters of
functional dupilumab in serum

| | ≥2 to <6 | | ≥2 to <6 | | ≥6 months | | ≥6 months | |
|---|---|---|---|---|---|---|---|---|
| | years of | | years of | | to <2 years | | to <2 years | |
| | age | | age | | of age | | of age | |
| | 3 mg/kg | | 6 mg/kg | | 3 mg/kg | | 6 mg/kg | |
| | (n = 9)^{†} | | (n = 10) | | (n = 10) | | (n = 10) | |
| | | Mean | | Mean | | Mean | | Mean |
| Parameter | n | (SD) | n | (SD) | n | (SD) | n | (SD) |
| Cₘₐₓ (mg/L) | 9 | 25.2 (7.44) | 10 | 49.8 (11.3) | 10 | 20.1 (6.81) | 10 | 46.1 (11.1) |
| Cₘₐₓ/dose | 9 | 8.39 (2.48) | 10 | 8.30 (1.89) | 10 | 6.70 (2.27) | 10 | 7.68 (1.86) |
| [mg/L/(mg/kg)] | | | | | | | | |
| tₘₐₓ (day)^{‡} | 9 | 1.92 (1.72-3.02) | 10 | 1.97 (1.87-7.82) | 10 | 1.95 (1.75-3.08) | 10 | 2.10 (1.80-7.99) |
| Cₗₐₛₜ (mg/L) | 9 | 6.64 (6.16) | 10 | 6.14 (4.69) | 10 | 5.64 (4.52) | 10 | 15.1 (9.48) |
| tₗₐₛₜ (day)^{‡} | 9 | 14.8 (6.79-28.0) | 10 | 26.5 (15.0-32.0) | 10 | 13.9 (6.88-16.8) | 10 | 16.0 (6.95-28.0) |
| AUCₗₐₛₜ (day · mg/L) | 9 | 215 (138) | 10 | 670 (197) | 10 | 133 (87.0) | 10 | 519 (304) |
| AUCₗₐₛₜ/dose | 9 | 71.5 (45.9) | 10 | 112 (32.8) | 10 | 44.3 (29.0) | 10 | 86.5 (50.6) |
| [day · mg/L/(mg/kg)] | | | | | | | | |

^{†}One patient in the ≥2 to <6 year 3 mg/kg dose group was excluded due to undetectable concentrations at all time points.
^{‡}Median (range).
AUCₗₐₛₜ, area under the curve from time zero to the last observed concentration; Cₘₐₓ, maximum concentration; Cₗₐₛₜ, last observed concentration; n, number of patients; SD, standard deviation; tₘₐₓ, time to maximum concentration; tₗₐₛₜ, time to last observed concentration.

### Efficacy

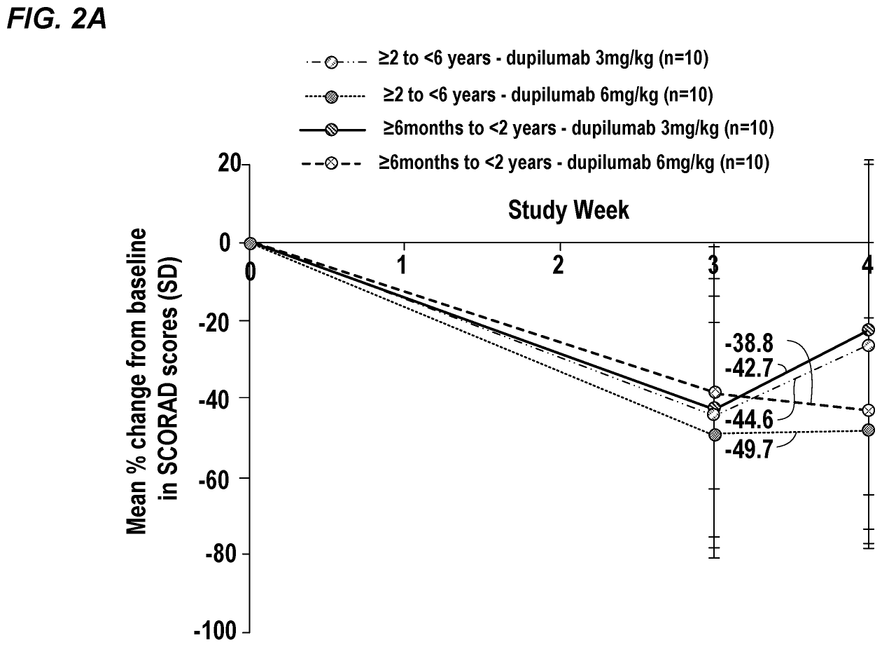

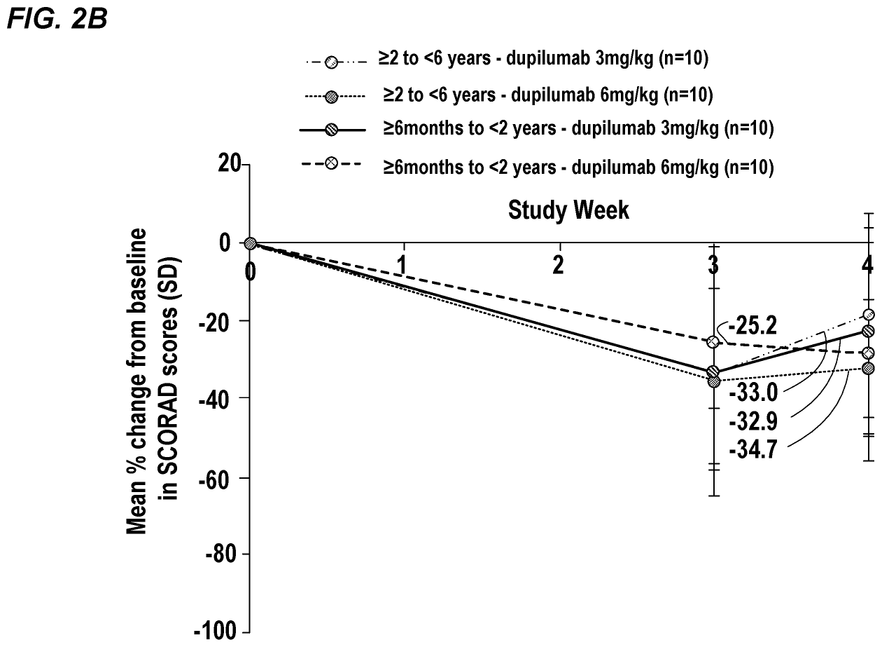

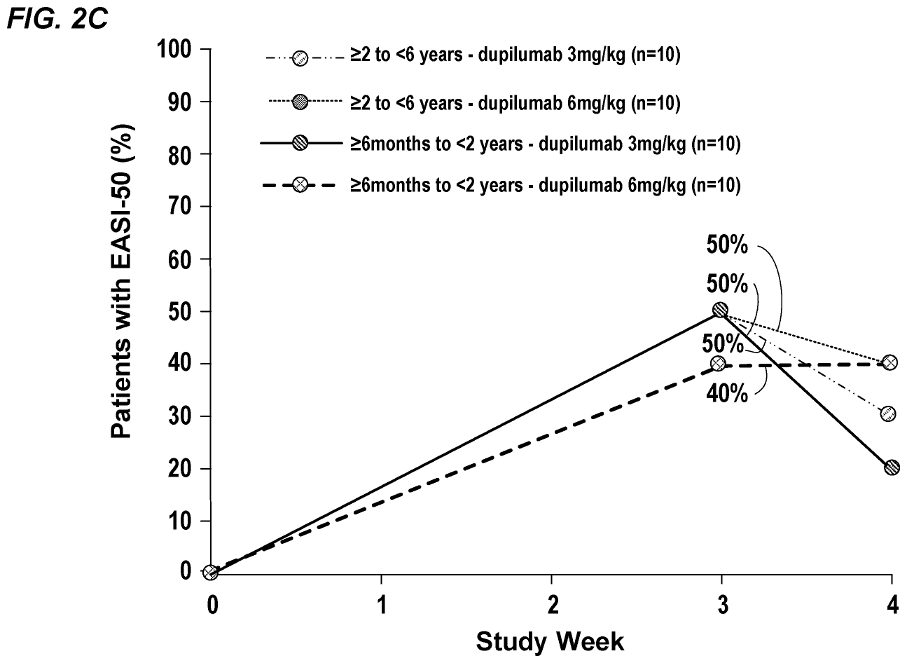

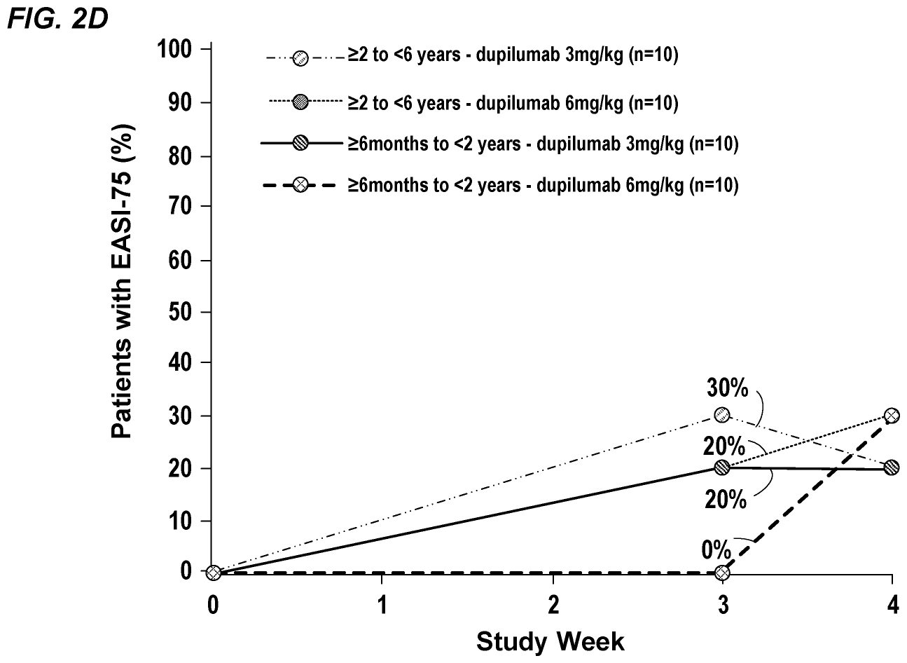

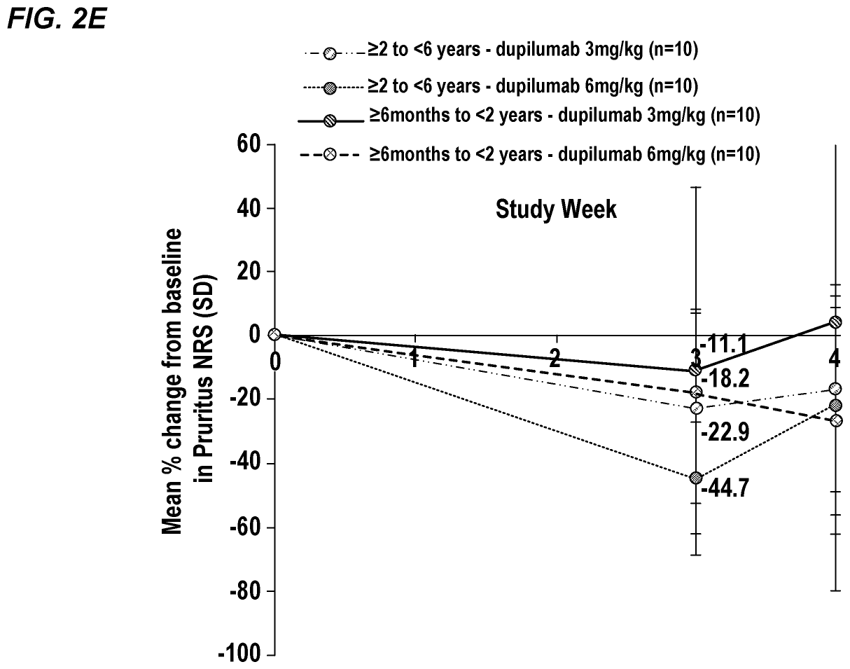

In the older cohort, both dupilumab doses led to improvements in clinical AD signs and symptoms at Week 3, as assessed by reductions from baseline in mean EASI, total SCORAD, SCORAD Visual Analog Scale (VAS) itch scores (Table 3, FIG. 2A-2B) and extent of BSA involvement. SCORAD VAS sleep scores also improved at Week 3, but only with the 6 mg/kg dose; the apparent lack of response with the 3 mg/kg dose was driven by 1 patient with outlier values (Table 3). EASI scores decreased by 44.6% with the 3 mg/kg and 49.7% with the 6 mg/kg dose (Table 3). Improvement in AD signs was also shown by the proportions of patients with EASI-50 (50% and 50%) and EASI-75 (30% and 20%) at Week 3 after the single dose of 3 and 6 mg/kg, respectively (Table 3; FIG. 2C-2D). Itch was also improved, as shown by mean reductions in caregiver-reported Peak Pruritus NRS of 22.9% and 44.7% from baseline at Week 3 for the 3 mg/kg and 6 mg/kg doses, respectively (Table 3, FIG. 2E).

AD clinical signs improved in both dose groups of the younger cohort. EASI scores decreased by a mean 42.7% and 38.8% at Week 3 with the 3 mg/kg and 6 mg/kg doses, respectively (Table 3, FIG. 2A). Total SCORAD scores, as well as SCORAD VAS scores for sleep and itch, and percentage of BSA affected also reduced with both dupilumab doses at Week 3 (Table 3 and FIG. 2B). The proportion of patients with EASI-50 was 50% and 40%, and with EASI-75 was 20% and 0% at Week 3 after the 3 mg/kg and 6 mg/kg doses, respectively while caregiver-reported Peak Pruritus NRS scores decreased by a mean of 11.1% and 18.2% (Table 3, FIG. 2C-2E).

At Week 4, the reduction in efficacy outcomes such as EASI, SCORAD, and caregiver-reported Peak Pruritus NRS scores, started to reverse, but was better sustained in the higher dose groups in both age groups. All efficacy outcomes were overall improved compared to baseline (Table 3, FIG. 2A-2E) and, in general, were numerically higher in the 6 mg/kg cohorts (Table 3).

TABLE 3
Efficacy outcomes

| | ≥2 to <6 years of age | | | | | | | | | | | |
|---|---|---|---|---|---|---|---|---|---|---|---|---|
| | Dupilumab 3 mg/kg | | | | | | Dupilumab 6 mg/kg | | | | | |
| | (n = 10) | | | | | | (n = 10) | | | | | |
| | Baseline | | Week 3 | | Week 4 | | Baseline | | Week 3 | | Week 4 | |
| EASI score, | 35.2 | (9.21) | 20.9 | (17.31) | 26.2 | (18.44) | 40.2 | (11.81) | 20.1 | (12.25) | 20.9 | (12.87) |
| mean (SD) | | | | | | | | | | | | |
| EASI score % | N/A | | −44.6 | (36.77) | −26.6 | (47.37) | 40.2 | (11.81) | −49.7 | (29.05) | −48.7 | (28.89) |
| change from | | | | | | | | | | | | |
| baseline, | | | | | | | | | | | | |
| mean (SD) | | | | | | | | | | | | |
| 95% CI of | N/A | | −70.9, | | −60.5, | | N/A | | −70.5, | | −69.3, | |
| mean | | | −18.3 | | 7.3 | | | | −29.0 | | −28.0 | |
| Total | 73.5 | (10.20) | 50.1 | (26.88) | 60.1 | (22.02) | 75.1 | (8.08) | 49.5 | (19.26) | 51.6 | (15.79) |
| SCORAD | | | | | | | | | | | | |
| score, | | | | | | | | | | | | |
| mean (SD) | | | | | | | | | | | | |
| Total | N/A | | −33.0 | (32.09) | −18.6 | (26.18) | N/A | | −34.7 | (23.18) | −31.9 | (17.45) |
| SCORAD % | | | | | | | | | | | | |
| change from | | | | | | | | | | | | |
| baseline, | | | | | | | | | | | | |
| mean (SD) | | | | | | | | | | | | |
| 95% CI of | | | −55.9, | | −37.4, | | | | −51.3, | | −44.4, | |
| mean | | | −10.0 | | 0.1 | | | | −18.1 | | −19.4 | |
| SCORAD | 6.3 | (2.54) | 5.5 | (3.87) | 5.1 | (3.76) | 6.3 | (2.82) | 3.2 | (2.39) | 4.0 | (3.01) |
| VAS sleep | | | | | | | | | | | | |
| score, mean | | | | | | | | | | | | |
| (SD) | | | | | | | | | | | | |
| SCORAD | N/A | | −2.8 | (92.32)^{†} | 11.8 | (136.08)^{†} | N/A | | −44.1 | (48.42) | −37.9 | (35.08) |
| VAS sleep | | | | | | | | | | | | |
| score % | | | | | | | | | | | | |
| change from | | | | | | | | | | | | |
| baseline, | | | | | | | | | | | | |
| mean (SD) | | | | | | | | | | | | |
| 95% CI of | | | −68.9, | | −85.5, | | | | −78.8, | | −63.0, | |
| mean | | | 63.2 | | 109.2 | | | | −9.5 | | −12.8 | |
| SCORAD | 8.4 | (1.18) | 6.0 | (3.29) | 7.2 | (2.81) | 7.6 | (1.78) | 4.2 | (1.92) | 5.2 | (1.84) |
| VAS itch | | | | | | | | | | | | |
| score, | | | | | | | | | | | | |
| mean (SD) | | | | | | | | | | | | |
| SCORAD | N/A | | −30.0 | (34.68) | −14.1 | (30.22) | N/A | | −44.5 | (26.04) | −27.1 | (35.17) |
| VAS itch | | | | | | | | | | | | |
| score % | | | | | | | | | | | | |
| change from | | | | | | | | | | | | |
| baseline, | | | | | | | | | | | | |
| mean (SD) | | | | | | | | | | | | |
| 95% CI of | | | −54.8, | | −35.7, | | | | −63.1, | | −52.2, | |
| mean | | | −5.2 | | 7.5 | | | | −25.8 | | −1.9 | |
| Patients with | N/A | | 0 | | 1 | (10) | N/A | | 0 | | 0 | |
| IGA 0 or 1, | | | | | | | | | | | | |
| n/N (%) | | | | | | | | | | | | |
| 95% CI of | | | 0, 30.85 | | 0.25, | | | | 0, | | 0, | |
| mean | | | | | 44.50 | | | | 30.85 | | 30.85 | |
| Patients with | N/A | | 5 | (50.0) | 3 | (30.0) | N/A | | 5 | (50.0) | 4 | (40.0) |
| EASI-50, n/N | | | | | | | | | | | | |
| (%) | | | | | | | | | | | | |
| 95% CI of | | | 18.71, | | 6.67, | | | | 18.71, | | 12.16, | |
| mean | | | 81.29 | | 65.25 | | | | 81.29 | | 73.76 | |
| Patients with | N/A | | 3 | (30.0) | 2 | (20.0) | N/A | | 2 | (20.0) | 3 | (30.0) |
| EASI-75, n/N | | | | | | | | | | | | |
| (%) | | | | | | | | | | | | |
| 95% CI of | | | 6.67, | | 2.52, | | | | 2.52, | | 6.67, | |
| mean | | | 65.25 | | 55.61 | | | | 55.61 | | 65.25 | |
| Caregiver- | 8.4 | (1.2) | 6.7 | (2.8) | 7.1 | (3.1) | 8.1 | (1.4) | 4.5 | (1.6) | 6.1 | (2.3) |
| reported Peak | | | | | | | | | | | | |
| Pruritus NRS | | | | | | | | | | | | |
| score, | | | | | | | | | | | | |
| mean (SD) | | | | | | | | | | | | |
| Caregiver- | N/A | | −22.9 | (29.9) | −16.7 | (32.5) | N/A | | −44.7 | (17.5) | −22.0 | (34.5) |
| reported Peak | | | | | | | | | | | | |
| Pruritus NRS | | | | | | | | | | | | |
| score, % | | | | | | | | | | | | |
| change from | | | | | | | | | | | | |
| baseline, | | | | | | | | | | | | |
| mean (SD) | | | | | | | | | | | | |
| % BSA | 58.1 | (11.09) | 38.1 | (20.74) | 42.2 | (20.95) | 67.5 | (16.05) | 39.5 | (17.46) | 37.2 | (22.03) |
| affected, | | | | | | | | | | | | |
| mean (SD) | | | | | | | | | | | | |
| % BSA | N/A | | −34.5 | (33.66) | −23.8 | (41.82) | N/A | | −40.8 | (25.72) | −43.6 | (29.56) |
| affected, % | | | | | | | | | | | | |
| change from | | | | | | | | | | | | |
| baseline, | | | | | | | | | | | | |
| mean (SD) | | | | | | | | | | | | |
| 95% CI of | | | −58.6, | | −53.8, | | | | −59.2, | | −64.7, | |
| mean | | | −10.4 | | 6.1 | | | | −22.4 | | −22.4 | |
| | ≥6 months to <2 years of age | | | | | | | | | | | |
| | Dupilumab 3 mg/kg | | | | | | Dupilumab 6 mg/kg | | | | | |
| | (n = 10) | | | | | | (n = 10) | | | | | |
| | Baseline | | Week 3 | | Week 4 | | Baseline | | Week 3 | | Week 4 | |
| EASI score, | 34.4 | (14.25) | 22.4 | (19.70) | 27.0 | (15.75) | 36.1 | (12.94) | 22.4 | (12.23) | 21.2 | (17.44) |
| mean (SD) | | | | | | | | | | | | |
| EASI score % | N/A | | −42.7 | (33.13) | −22.4 | (42.52) | N/A | | −38.8 | (24.98) | −43.2 | (35.55) |
| change from | | | | | | | | | | | | |
| baseline, | | | | | | | | | | | | |
| mean (SD) | | | | | | | | | | | | |
| 95% CI of | N/A | | −66.4, | | −52.9, | | N/A | | −56.7, | | −68.6, | |
| mean | | | −19.0 | | 8.0 | | | | −20.9 | | −17.8 | |
| Total | 69.8 | (13.10) | 48.7 | (23.21) | 55.2 | (21.34) | 75.9 | (11.74) | 56.2 | (13.84) | 54.9 | (24.50) |
| SCORAD | | | | | | | | | | | | |
| score, | | | | | | | | | | | | |
| mean (SD) | | | | | | | | | | | | |
| Total | N/A | | −32.9 | (23.76) | −22.4 | (26.44) | N/A | | −25.2 | (17.18) | −28.1 | (27.84) |
| SCORAD % | | | | | | | | | | | | |
| change from | | | | | | | | | | | | |
| baseline, | | | | | | | | | | | | |
| mean (SD) | | | | | | | | | | | | |
| 95% CI of | | | −49.9, | | −41.3, | | | | −37.5, | | −48.1, | |
| mean | | | −15.9 | | −3.5 | | | | −12.9 | | −8.2 | |
| SCORAD | 6.0 | (2.83) | 4.2 | (2.96) | 4.3 | (3.00) | 7.8 | (1.90) | 4.8 | (2.52) | 6.0 | (3.49) |
| VAS sleep | | | | | | | | | | | | |
| score, mean | | | | | | | | | | | | |
| (SD) | | | | | | | | | | | | |
| SCORAD | N/A | | −32.8 | (39.83) | −25.2 | (53.14) | N/A | | −34.5 | (39.75) | −24.3 | (46.68) |
| VAS sleep | | | | | | | | | | | | |
| score % | | | | | | | | | | | | |
| change from | | | | | | | | | | | | |
| baseline, | | | | | | | | | | | | |
| mean (SD) | | | | | | | | | | | | |
| 95% CI of | | | −61.3, | | −63.2, | | | | −62.9, | | −57.7, | |
| mean | | | −4.3 | | 12.9 | | | | −6.1 | | 9.1 | |
| SCORAD | 7.5 | (2.40) | 5.1 | (2.31) | 5.9 | (2.57) | 8.2 | (1.42) | 6.3 | (2.77) | 6.4 | (3.20) |
| VAS itch | | | | | | | | | | | | |
| score, | | | | | | | | | | | | |
| mean (SD) | | | | | | | | | | | | |
| SCORAD | N/A | | −27.0 | (33.73) | −11.7 | (48.44) | N/A | | −19.2 | (43.49) | −24.7 | (31.09) |
| VAS itch | | | | | | | | | | | | |
| score % | | | | | | | | | | | | |
| change from | | | | | | | | | | | | |
| baseline, | | | | | | | | | | | | |
| mean (SD) | | | | | | | | | | | | |
| 95% CI of | | | −51.1, | | −46.4, | | | | −50.4, | | −47.0, | |
| mean | | | −2.9 | | 22.9 | | | | 11.9 | | −2.5 | |
| Patients with | N/A | | 0 | | 1 | (10.0) | N/A | | 0 | | 1 | (10.0) |
| IGA 0 or 1, | | | | | | | | | | | | |
| n/N (%) | | | | | | | | | | | | |
| 95% CI of | | | 0, | | 0.25, | | | | 0, | | 0.25, | |
| mean | | | 30.85 | | 44.50 | | | | 30.85 | | 44.50 | |
| Patients with | N/A | | 5 | (50.0) | 2 | (20.0) | N/A | | 4 | (40.0) | 4 | (40.0) |
| EASI-50, n/N | | | | | | | | | | | | |
| (%) | | | | | | | | | | | | |
| 95% CI of | | | 18.71, | | 2.52, | | | | 12.16, | | 12.16, | |
| mean | | | 81.29 | | 55.61 | | | | 73.76 | | 73.76 | |
| Patients with | N/A | | 2 | (20.0) | 2 | (20.0) | N/A | | 0 | | 3 | (30.0) |
| EASI-75, n/N | | | | | | | | | | | | |
| (%) | | | | | | | | | | | | |
| 95% CI of | | | 2.52, | | 2.52, | | | | 0, | | 6.67, | |
| mean | | | 55.61 | | 55.61 | | | | 30.85 | | 65.25 | |
| Caregiver- | 7.6 | (2.5) | 5.8 | (2.2) | 6.5 | (2.1) | 8.5 | (0.7) | 7.0 | (2.4) | 6.3 | (3.2) |
| reported Peak | | | | | | | | | | | | |
| Pruritus NRS | | | | | | | | | | | | |
| score, | | | | | | | | | | | | |
| mean (SD) | | | | | | | | | | | | |
| Caregiver- | N/A | | −11.1 | (57.5)^{‡} | 4.1 | (84.2)^{‡} | N/A | | −18.2 | (26.4) | −26.7 | (35.5) |
| reported Peak | | | | | | | | | | | | |
| Pruritus NRS | | | | | | | | | | | | |
| score, % | | | | | | | | | | | | |
| change from | | | | | | | | | | | | |
| baseline, | | | | | | | | | | | | |
| mean (SD) | | | | | | | | | | | | |
| % BSA | 55.3 | (25.66) | 46.2 | (30.41) | 50.1 | (29.54) | 57.9 | (21.37) | 35.0 | (18.79) | 33.0 | (24.70) |

| | | | | | | | | | | |
|---|---|---|---|---|---|---|---|---|---|---|
| affected, | | | | | | | | | | |
| mean (SD) | | | | | | | | | | |
| % BSA | N/A | −22.1 | (31.57) | −12.3 | (38.38) | N/A | −40.3 | (21.21) | −44.8 | (31.75) |

| | | | | |
|---|---|---|---|---|
| affected, % | | | | |
| change from | | | | |
| baseline, | | | | |
| mean (SD) | | | | |
| 95% CI of | −44.6, | −39.8, | −55.4, | −67.5, |
| mean | 0.5 | 15.1 | −25.1 | −22.0 |

^{†}The apparent lack of efficacy in this age and dose subgroup is due to an outlier patient with a SCORAD VAS sleep loss score of 1.8, 5.6, 8.4 at baseline, Week 3, and Week 4, respectively, with a corresponding percentage change from baseline of 211.1% and 366.7% for Weeks 3 and 4, respectively.
^{‡}The apparent lack of efficacy in this age and dose subgroup is due to an outlier patient with a caregiver-reported Peak Pruritus NRS score of 3, 7, and 10 at baseline, Week 3, and Week 4, respectively, with a corresponding percentage change from baseline of 133.3% and 233.3% for Weeks 3 and 4, respectively.

### Safety

In the older cohort, five TEAEs were reported in the 3 mg/kg group and three TEAEs in the 6 mg/kg group. The incidence of TEAEs was similar across treatment groups (Table 4), and the severity of all TEAEs was mild or moderate. One SAE (anaphylactic reaction) was reported with dupilumab 3 mg/kg in a patient with history of anaphylaxis to peanuts and documented egg, peanut, dairy, and soy food allergies, immediately after a meal suspected to contain nuts. This SAE was not considered treatment-related based on the history of food allergies and anaphylaxis, as well as the temporal onset of the event after dosing. No AE was reported in more than 1 patient per treatment group, and none were considered treatment-related. No conjunctivitis or other superficial eye disorder, herpes viral infection, or injection-site reactions were reported.

The number of TEAEs was higher in the younger cohorts (11 in each dose group; Table 4). Most were mild to moderate. Two patients in the 6 mg/kg dose group had an AE related to study drug (diarrhea and injection-site erythema), neither of which was severe or serious. One patient in the 3 mg/kg dose group had a serious TEAE, an anaphylactic reaction immediately after eating crab and >2 weeks after dosing, so deemed unrelated to dupilumab. Besides nasopharyngitis, no AE was reported in >1 patient in either treatment group (Table 3). No conjunctivitis or other superficial eye disorders or herpes viral infections were reported. No deaths occurred during the study.

TABLE 4
Safety assessment at Week 4

| | ≥2 to <6 years | | ≥6 months to | |
|---|---|---|---|---|
| | of age | | <2 years of age | |
| | Dupilumab | Dupilumab | Dupilumab | Dupilumab |
| | 3 mg/kg | 6 mg/kg | 3 mg/kg | 6 mg/kg |
| | (n = 10) | (n = 10) | (n = 10) | (n = 10) |
| TEAEs, n | | | | |
| Total number of TEAEs | 5 | 3 | 11 | 11 |
| Total number of serious TEAEs | 1 | 0 | 1 | 0 |
| Total number of TEAEs related to treatment | 0 | 0 | 0 | 2 |
| Patients with TEAEs, n (%) | | | | |
| ≥1 TEAE | 3 (30.0) | 2 (20.0) | 7 (70.0) | 7 (70.0) |
| ≥1 serious TEAE | 1 (10.0) | 0 | 1 (10.0) | 0 |
| ≥1 severe TEAE | 0 | 0 | 1 (10.0) | 0 |
| Any infection (SOC) | 2 (20.0) | 1 (10.0) | 3 (30.0) | 4 (40.0) |
| Skin infection^{†} | 1 (10.0) | 0 | 2 (20.0) | 1 (10.0) |
| Non-herpetic skin infection^{†} | 1 (10.0) | 0 | 2 (20.0) | 1 (10.0) |
| Impetigo (PT) | 1 (10.0) | 0 | 1 (10.0) | 1 (10.0) |
| Folliculitis (PT) | 0 | 0 | 1 (10.0) | 0 |
| Herpes viral infections (HLT) | 0 | 0 | 0 | 0 |
| Injection-site reactions (HLT) | 0 | 0 | 0 | 1 (10.0) |
| Injection-site erythema (PT) | 0 | 0 | 0 | 1 (10.0) |
| TEAEs (PT), n (%)^{‡} | | | | |
| Nasopharyngitis | 1 (10.0) | 1 (10.0) | 1 (10.0) | 2 (20.0) |
| Diarrhea | 0 | 0 | 1 (10.0) | 1 (10.0) |
| Upper respiratory tract infection | 0 | 0 | 1 (10.0) | 1 (10.0) |
| Urticaria | 0 | 0 | 1 (10.0) | 1 (10.0) |
| Dermatitis atopic | 1 (10.0) | 0 | 0 | 1 (10.0) |
| Cough | 0 | 1 (10.0) | 0 | 1 (10.0) |
| Pyrexia | 1 (10.0) | 0 | 1 (10.0) | 0 |
| Anaphylactic reaction | 1 (10.0) | 0 | 1 (10.0) | 0 |
| Constipation | 0 | 0 | 1 (10.0) | 0 |
| Folliculitis | 0 | 0 | 1 (10.0) | 0 |
| Joint swelling | 0 | 0 | 1 (10.0) | 0 |
| Lacrimation increased | 0 | 0 | 0 | 1 (10.0) |
| Skin abrasion | 0 | 1 (10.0) | 0 | 0 |
| Teething | 0 | 0 | 1 (10.0) | 0 |
| Thrombocytosis | 0 | 0 | 0 | 1 (10.0) |
| Conjunctivitis | 0 | 0 | 0 | 0 |
| Herpes simplex | 0 | 0 | 0 | 0 |

Adverse events reported according to MedDRA PTs unless otherwise specified.
^{†}Adjudicated.
^{‡}Includes all MedDRA PTs reported in ≥10% of patients in any treatment group of the study.
HLT, MedDRA High Level Term;
MedDRA, Medical Dictionary for Regulatory Activities;
PT, MedDRA Preferred Term;
SOC, MedDRA System Organ Class;
TEAE, treatment-emergent adverse event.

### Biomarker Analysis

In both older and younger patients, dupilumab at both doses markedly suppressed serum TARC and total IgE (Table 5). Blood eosinophil count at Week 4 marginally increased in older patients but decreased in younger patients in the 3 mg/kg groups; it remained unchanged after 6 mg/kg dupilumab in the older and younger cohorts (Table 5).

TABLE 5
Blood serum biomarkers

| | ≥2 to <6 years of age | | | | | | | |
|---|---|---|---|---|---|---|---|---|
| | Dupilumab 3 mg/kg | | | | Dupilumab 6 mg/kg | | | |
| | (n = 10) | | | | (n = 10) | | | |
| | Baseline | | Week 4 | | Baseline | | Week 4 | |
| n | 10 | | 10 | | 10 | | 10 | |
| CCL17/TARC | 6340.0 | (2830.0, 18100.0) | 4625.0 | (1680.0, 8420.0) | 5750.0 | (1690.0, 9610.0) | 730.0 | (385.0, 1820.0) |
| concentration, | | | | | | | | |
| median (Q1, Q3), pg/mL | | | | | | | | |
| CCL17/TARC, | N/A | | −26.4 | (−53.5, 7.1) | N/A | | −66.4 | (−90.4, −59.0) |
| median % | | | | | | | | |
| change from baseline | | | | | | | | |
| (Q1, Q3), pg/mL | | | | | | | | |
| n | 9 | | 9 | | 10 | | 10 | |
| Total IgE concentration, | 3990.0 | (1590.0, 8910.0) | 5000.0 | (2460.0, 7230.0) | 3070.0 | (708.0, 11100.0) | 2275.0 | (259.0, 6870.0) |
| median (Q1, Q3), | | | | | | | | |
| IU/mL | | | | | | | | |
| Total IgE, median % | N/A | | −20.38 | (−28.24, −6.01) | N/A | | −32.85 | (−44.96, −11.52) |
| change from baseline | | | | | | | | |
| (Q1, Q3), IU/mL | | | | | | | | |
| n | 10 | | 10 | | 10 | | 9 | |
| Blood eosinophil count, | 1.05 | (0.60, 2.30) | 1.40 | (1.20, 2.20) | 0.80 | (0.40, 1.30) | 0.90 | (0.60, 1.70) |
| median | | | | | | | | |
| (Q1, Q3), ×10⁹/L | | | | | | | | |
| Blood eosinophil count, | N/A | | 0.10 | (−0.40, 0.80) | N/A | | 0.00 | (−0.20, 0.10) |
| median change from | | | | | | | | |
| baseline | | | | | | | | |
| (Q1, Q3), ×10⁹/L | | | | | | | | |
| | ≥6 months to <2 years of age | | | | | | | |
| | Dupilumab 3 mg/kg | | | | Dupilumab 6 mg/kg | | | |
| | (n = 10) | | | | (n = 10) | | | |
| | Baseline | | Week 4 | | Baseline | | Week 4 | |
| n | 10 | | 10 | | 4 | | 10 | |
| CCL17/TARC | 1625.0 | (546.0, 6720.0) | 3045.0 | (1650.0, 6520.0) | 2595.0 | (1303.0, 6610.0) | 1700.0 | (431.0, 5190.0) |
| concentration, | | | | | | | | |
| median (Q1, Q3), pg/mL | | | | | | | | |
| CCL17/TARC, | N/A | | −21.1 | (−56.4, 464.4) | N/A | | −57.6 | (−77.2, 10.5) |
| median % | | | | | | | | |
| change from baseline | | | | | | | | |
| (Q1, Q3), pg/mL | | | | | | | | |
| n | 10 | | 9 | | 10 | | 8 | |
| Total IgE concentration, | 1680.0 | (509.0, 3920.0) | 944.0 | (439.0, 2860.0) | 500.0 | (165.0, 3610.0) | 631.0 | (82.30, 4555.0) |
| median (Q1, Q3), | | | | | | | | |
| IU/mL | | | | | | | | |
| Total IgE, median % | N/A | | −23.25 | (−27.04, −13.79) | N/A | | −37.27 | (−53.58, −5.43) |
| change from baseline | | | | | | | | |
| (Q1, Q3), IU/mL | | | | | | | | |
| n | 9 | | 9 | | 10 | | 9 | |
| Blood eosinophil count, | 1.60 | (0.40, 1.90) | 1.10 | (0.90, 1.60) | 1.45 | (0.80, 2.60) | 1.00 | (0.80, 1.60) |

| | | | | | | |
|---|---|---|---|---|---|---|
| median | | | | | | |
| (Q1, Q3), ×10⁹/L | | | | | | |
| Blood eosinophil count, | N/A | −0.10 | (−0.70, 0.20) | N/A | 0.00 | (−0.50, 0.20) |
| median change from | | | | | | |
| baseline | | | | | | |
| (Q1, Q3), ×10⁹/L | | | | | | |

N/A, not applicable; Q1, first quartile; Q3, third quartile; TARC, thymus and activation-regulated chemokine.

### Concomitant TCS Use for AD

The majority of patients in both the younger (80% and 60% with 3 mg/kg and 6 mg/kg, respectively) and older cohort (90% and 80% with 3 mg/kg and 6 mg/kg, respectively) used concomitant TCS for AD during the study (Table 6). Most of the concomitant TCS used was of moderate potency (group II); none of the patients used very potent (group IV) TCS during the study (Table 6).

TABLE 6
Concomitant TCS use for AD

| | ≥2 to <6 years of age | | ≥6 months to <2 years of age | |
|---|---|---|---|---|
| | Dupilumab | Dupilumab | Dupilumab | Dupilumab |
| | 3 mg/kg | 6 mg/kg | 3 mg/kg | 6 mg/kg |
| n (%) | (n = 10) | (n = 10) | (n = 10) | (n = 10) |
| Patients with | 9 (90.0) | 8 (80.0) | 8 (80.0) | 6 (60.0) |
| concomitant TCS | | | | |
| use for AD | | | | |
| No concomitant | 1 (10.0) | 2 (20.0) | 2 (20.0) | 4 (40.0) |
| TCS usage | | | | |
| Any use | 9 (90.0) | 8 (80.0) | 8 (80.0) | 6 (60.0) |
| Mildly potent | 1 (10.0) | 1 (10.0) | 4 (40.0) | 2 (20.0) |
| TCS (group I) | | | | |
| Moderately | 7 (70.0) | 7 (70.0) | 7 (70.0) | 3 (30.0) |
| potent TCS | | | | |
| (group II) | | | | |
| Potent TCS | 3 (30.0) | 4 (40.0) | 3 (30.0) | 4 (40.0) |
| (group III) | | | | |
| Very potent | 0 | 0 | 0 | 0 |
| TCS (group | | | | |
| IV) | | | | |

AD, atopic dermatitis;
TCS, topical corticosteroid(s).

### Discussion

Both dose groups in both age cohorts experienced improvement in AD signs and symptoms as measured by EASI, total SCORAD, SCORAD VAS for sleep and itch, and caregiver-reported Peak Pruritus NRS scores at Week 3. However, there was a trend toward slightly better responses in the older versus younger age cohort, particularly when comparing the 6 mg/kg dose groups. At Week 4, not all benefits were sustained in either age cohort, and loss of efficacy was more pronounced in the lower dose group. These findings suggest a potential benefit of repeated administration.

Dupilumab exhibits non-linear, target-mediated PK as previously characterized in adult and adolescent patients with moderate-to-severe AD and supported by the greater than dose-proportional increases in AUC observed in the current study. Slightly lower exposures were observed in the younger patients than in the older patients at the same mg/kg dose level in this study. It has been described previously for monoclonal antibodies in general, and for dupilumab, in particular, that clearance of drug does not scale linearly with body weight. This manifests as faster clearance on a per kilogram of total body weight in smaller individuals. Accordingly, using the same mg/kg dose regimen across a wide weight range in a pediatric population over-corrects dose for the impact of body weight and results in lower exposures in younger patients.

Maintaining sufficient concentrations of antagonistic antibodies like dupilumab is important for blocking target pathways throughout the intended duration of treatment. When administered under similar multiple dosing scenarios, the faster elimination on a per kilogram of total body weight basis may require larger body weight-normalized doses in younger populations to maintain similar trough concentrations. This is supported by the fact that a single dupilumab 300 mg dose in adults, equivalent to ≤5 mg/kg in a ≥60 kg adult, leads to a similar exposure to the 6 mg/kg dose used in this study. Other mechanisms such as higher levels of IL-13 gene expression in non-lesional AD skin in children than adults may also contribute to more rapid removal of drug by receptor-mediated pathways in young children.

Overall, single-dose dupilumab treatment suppressed serum type 2 inflammatory biomarkers TARC and total IgE, consistent with findings in adolescents and adults, suggesting a shared underlying mechanism of inflammation involving interleukin-4 and interleukin-13 as mediators. Indeed, even pediatric patients aged <2 years with recent-onset AD have shown a strong Th2-skewed immune response. There was no clear effect of single-dose dupilumab on blood eosinophil count.

The safety profile of dupilumab in children aged ≥6 months to <6 years was comparable to that seen in adults, adolescents and children >6 years. There were no dupilumab-related events of serious infection or systemic hypersensitivity.

The greater number of TEAEs in the younger cohort appeared not to be driven by any particular event, and majority of events were deemed unrelated to dupilumab. The acceptable safety profile was also reassuring, given theoretical concerns about use of immunomodulating treatment in young children. Supporting safety data after multiple-dose treatment would differentiate use of a targeted immunomodulator such as dupilumab from broad immunosuppressants currently used off-label in young children.

### CONCLUSION

A single subcutaneous dose of dupilumab in children ≥6 months to <6 years with severe AD yielded substantial clinical benefit in reducing signs and symptoms of AD, with no clear dose-response observed at Week 3. However, at Week 4, improvements in most efficacy responses started to reverse, particularly in the lower dose group. There was a trend toward slightly higher exposure and efficacy in the older (≥2 to <6 years) versus younger age group (≥6 months to <2 years). Dupilumab was generally well tolerated in this pediatric population, and its safety profile was similar to that in adults, adolescents and children >6 years.

TABLE 7
Sequences

| SEQ | | |
|---|---|---|
| ID NO | Sequence | Description |
| 1 | EVQLVESGGGLEQPGGSLRLSCAGSGFTFRDYAMTWVRQAPGKGLE | Dupilumab HCVR |
| | WVSSISGSGGNTYYADSVKGRFTISRDNSKNTLYLQMNSLRAEDTA | amino acid sequence |
| | VYYCAKDRLSITIRPRYYGLDVWGQGTTVTVS | |
| 2 | DIVMTQSPLSLPVTPGEPASISCRSSQSLLYSIGYNYLDWYLQKSGQS | Dupilumab LCVR |
| | PQLLIYLGSNRASGVPDRFSGSGSGTDFTLKISRVEAEDVGFYYCMQ | amino acid sequence |
| | ALQTPYTFGQGTKLEIK | |
| 3 | GFTFRDYA | Dupilumab HCDR1 |
| | | amino acid sequence |
| 4 | ISGSGGNT | Dupilumab HCDR2 |
| | | amino acid sequence |
| 5 | AKDRLSITIRPRYYGLDV | Dupilumab HCDR3 |
| | | amino acid sequence |
| 6 | QSLLYSIGYNY | Dupilumab LCDR1 |
| | | amino acid sequence |
| 7 | LGS | Dupilumab LCDR2 |
| | | amino acid sequence |
| 8 | MQALQTPYT | Dupilumab LCDR3 |
| | | amino acid sequence |
| 9 | EVQLVESGGGLEQPGGSLRLSCAGSGFTFRDYAMTWVRQAPGKGLE | Dupilumab heavy |
| | WVSSISGSGGNTYYADSVKGRFTISRDNSKNTLYLQMNSLRAEDTA | chain amino acid |
| | VYYCAKDRLSITIRPRYYGLDVWGQGTTVTVSSASTKGPSVFPLAPC | sequence |
| | SRSTSESTAALGCLVKDYFPEPVTVSWNSGALTSGVHTFPAVLQSSG | |
| | LYSLSSVVTVPSSSLGTKTYTCNVDHKPSNTKVDKRVESKYGPPCPP | |
| | CPAPEFLGGPSVFLFPPKPKDTLMISRTPEVTCVVVDVSQEDPEVQFN | |
| | WYVDGVEVHNAKTKPREEQFNSTYRVVSVLTVLHQDWLNGKEYK | |
| | CKVSNKGLPSSIEKTISKAKGQPREPQVYTLPPSQEEMTKNQVSLTCL | |
| | VKGFYPSDIAVEWESNGQPENNYKTTPPVLDSDGSFFLYSRLTVDKS | |
| | RWQEGNVFSCSVMHEALHNHYTQKSLSLSLGK | |
| 10 | DIVMTQSPLSLPVTPGEPASISCRSSQSLLYSIGYNYLDWYLQKSGQS | Dupilumab light |
| | PQLLIYLGSNRASGVPDRFSGSGSGTDFTLKISRVEAEDVGFYYCMQ | chain amino acid |
| | ALQTPYTFGQGTKLEIKRTVAAPSVFIFPPSDEQLKSGTASVVCLLNN | sequence |
| | FYPREAKVQWKVDNALQSGNSQESVTEQDSKDSTYSLSSTLTLSKA | |
| | DYEKHKVYACEVTHQGLSSPVTKSFNRGEC | |
| 11 | MKVLQEPTCVSDYMSISTCEWKMNGPTNCSTELRLLYQLVFLLSEA | Human IL-4Rα |
| | HTCIPENNGGAGCVCHLLMDDVVSADNYTLDLWAGQQLLWKGSF | |
| | KPSEHVKPRAPGNLTVHTNVSDTLLLTWSNPYPPDNYLYNHLTYAV | |
| | NTWSENDPADFRIYNVTYLEPSLRIAASTLKSGISYRARVRAWAQCY | |
| | NTTWSEWSPSTKWHNSYREPFEQH | |
| 12 | EIVLTQSPGTLSLSPGERATLSCRASQSVSNSYLAWYQQKPGQAPRL | SCB-VL-39 |
| | LIFGASSRATGIPDRFSGSGSGTDFTLTISRLEPEDFAVYYCQQYGSSP | |
| | PWTFGQGTKVEIK | |
| 13 | EIVLTQSPGTLSLSPGERATLSCRASQSVSSSYLAWYQQKPGQAPRLL | SCB-VL-40 |
| | IYGASSRATGIPDRFSGSGSGTDFTLTISRLEPEDFAVYYCQQYGSSPP | |
| | WTFGQGTKVEIK | |
| 14 | EIVLTQSPGTLSLSPGERATLSCRASQSVSSSYLAWYQQKPGQAPRLL | SCB-VL-41 |
| | IFGASSRAPGIPDRFSGSGSGTDFTLTISRLEPEDFAVYYCQQYGSSPP | |
| | WTFGQGTKVEIK | |
| 15 | EIVLTQSPGTLSLSPGERATLSCRASQSVSNSYLAWYQQKPGQAPRL | SCB-VL-42 |
| | LIYGASSRATGIPDRFSGSGSGTDFTLTISRLEPEDFAVYYCQQYGSSP | |
| | PWTFGQGTKVEIK | |
| 16 | EIVLTQSPGTLSLSPGERATLSCRASQSVSNSYLAWYQQKPGQAPRL | SCB-VL-43 |
| | LIFGASSRAPGIPDRFSGSGSGTDFTLTISRLEPEDFAVYYCQQYGSSP | |
| | PWTFGQGTKVEIK | |
| 17 | EIVLTQSPGTLSLSPGERATLSCRASQSVSSSYLAWYQQKPGQAPRLL | SCB-VL-44 |
| | IYGASSRAPGIPDRFSGSGSGTDFTLTISRLEPEDFAVYYCQQYGSSPP | |
| | WTFGQGTKVEIK | |
| 18 | EIVLTQSPGTLSLSPGERATLSCRASQSVSSSYLAWYQQKPGQAPRLL | SCB-VL-45 |
| | IFGASSRATGIPDRFSGSGSGTDFTLTISRLEPEDFAVYYCQQYDHSPP | |
| | WTFGQGTKVEIK | |
| 19 | EIVLTQSPGTLSLSPGERATLSCRASQSVSSSYLAWYQQKPGQAPRLL | SCB-VL-46 |
| | IFGASSRATGIPDRFSGSGSGTDFTLTISRLEPEDFAVYYCQQYGSSAG | |
| | WTFGQGTKVEIK | |
| 20 | EIVLTQSPGTLSLSPGERATLSCRASQSVSSSYLAWYQQKPGQAPRLL | SCB-VL-47 |
| | IFGASSRATGIPDRFSGSGSGTDFTLTISRLEPEDFAVYYCQQYDHSA | |
| | GWTFGQGTKVEIK | |
| 21 | EIVLTQSPGTLSLSPGERATLSCRASQSVSNSYLAWYQQKPGQAPRL | SCB-VL-48 |
| | LIFGASSRATGIPDRFSGSGSGTDFTLTISRLEPEDFAVYYCQQYDHSP | |
| | PWTFGQGTKVEIK | |
| 22 | EIVLTQSPGTLSLSPGERATLSCRASQSVSSSYLAWYQQKPGQAPRLL | SCB-VL-49 |
| | IYGASSRATGIPDRFSGSGSGTDFTLTISRLEPEDFAVYYCQQYDHSPP | |
| | WTFGQGTKVEIK | |
| 23 | EIVLTQSPGTLSLSPGERATLSCRASQSVSSSYLAWYQQKPGQAPRLL | SCB-VL-50 |
| | IFGASSRAPGIPDRFSGSGSGTDFTLTISRLEPEDFAVYYCQQYDHSPP | |
| | WTFGQGTKVEIK | |
| 24 | EIVLTQSPGTLSLSPGERATLSCRASQSVSSSYLAWYQQKPGQAPRLL | SCB-VL-51 |
| | IYGASSRAPGIPDRFSGSGSGTDFTLTISRLEPEDFAVYYCQQYDHSA | |
| | GWTFGQGTKVEIK | |
| 25 | EIVLTQSPGTLSLSPGERATLSCRASQSVSNSYLAWYQQKPGQAPRL | SCB-VL-52 |
| | LIFGASSRAPGIPDRFSGSGSGTDFTLTISRLEPEDFAVYYCQQYDHSA | |
| | GWTFGQGTKVEIK | |
| 26 | EIVLTQSPGTLSLSPGERATLSCRASQSVSNSYLAWYQQKPGQAPRL | SCB-VL-53 |
| | LIYGASSRATGIPDRFSGSGSGTDFTLTISRLEPEDFAVYYCQQYDHS | |
| | AGWTFGQGTKVEIK | |
| 27 | EIVLTQSPGTLSLSPGERATLSCRASQSVSSSYLAWYQQKPGQAPRLL | SCB-VL-54 |
| | IFGASSRAPGIPDRFSGSGSGTDFTLTISRLEPEDFAVYYCQQYDHSA | |
| | GWTFGQGTKVEIK | |
| 28 | EIVLTQSPGTLSLSPGERATLSCRASQSVSSSYLAWYQQKPGQAPRLL | SCB-VL-55 |
| | IYGASSRATGIPDRFSGSGSGTDFTLTISRLEPEDFAVYYCQQYDHSA | |
| | GWTFGQGTKVEIK | |
| 29 | EIVLTQSPGTLSLSPGERATLSCRASQSVSNSYLAWYQQKPGQAPRL | SCB-VL-56 |
| | LIFGASSRATGIPDRFSGSGSGTDFTLTISRLEPEDFAVYYCQQYDHSA | |
| | GWTFGQGTKVEIK | |
| 30 | EIVLTQSPGTLSLSPGERATLSCRASQSVSSSYLAWYQQKPGQAPRLL | SCB-VL-57 |
| | IFGASSRATGIPDRFSGSGSGTDFTLTISRLEPEDFAVYYCQQYGSSPP | |
| | WTFGQGTKVEIK | |
| 31 | EIVLTQSPGTLSLSPGERATLSCRASQSVSNSYLAWYQQKPGQAPRL | SCB-VL-58 |
| | LIYGASSRAPGIPDRFSGSGSGTDFTLTISRLEPEDFAVYYCQQYDHS | |
| | AGWTFGQGTKVEIK | |
| 32 | EVQLVESGGGLVHPGGSLRLSCAGSGFTFSRNAMFWVRQAPGKGLE | SCB-VH-59 |
| | WVSGIGTGGATNYADSVKGRFTISRDNAKNSLYLQMNSLRAEDMA | |
| | VYYCARGRYYFDYWGQGTLVTVSS | |
| 33 | EVQLVQSGGGLVQPGGSLRLSCAGSGFTFSRNAMFWVRQAPGKGLE | SCB-VH-60 |
| | WVSGIGTGGATNYADSVKGRFTISRDNAKNSLYLQMNSLRAEDMA | |
| | VYYCARGRYYFDYWGQGTLVTVSS | |
| 34 | EVQLVQSGGGLVHPGGSLRLSCAASGFTFSRNAMFWVRQAPGKGLE | SCB-VH-61 |
| | WVSGIGTGGATNYADSVKGRFTISRDNAKNSLYLQMNSLRAEDMA | |
| | VYYCARGRYYFDYWGQGTLVTVSS | |
| 35 | EVQLVQSGGGLVHPGGSLRLSCAGSGFTFSRNAMFWVRQAPGKGLE | SCB-VH-62 |
| | WVSGIGTGGATSYADSVKGRFTISRDNAKNSLYLQMNSLRAEDMA | |
| | VYYCARGRYYFDYWGQGTLVTVSS | |
| 36 | EVQLVQSGGGLVHPGGSLRLSCAGSGFTFSRNAMFWVRQAPGKGLE | SCB-VH-63 |
| | WVSGIGTGGATNYADSVKGRFTISRDNAKNSLYLQMNSLRAEDTAV | |
| | YYCARGRYYFDYWGQGTLVTVSS | |
| 37 | EVQLVESGGGLVQPGGSLRLSCAGSGFTFSRNAMFWVRQAPGKGLE | SCB-VH-64 |
| | WVSGIGTGGATNYADSVKGRFTISRDNAKNSLYLQMNSLRAEDMA | |
| | VYYCARGRYYFDYWGQGTLVTVSS | |
| 38 | EVQLVESGGGLVHPGGSLRLSCAASGFTFSRNAMFWVRQAPGKGLE | SCB-VH-65 |
| | WVSGIGTGGATNYADSVKGRFTISRDNAKNSLYLQMNSLRAEDMA | |
| | VYYCARGRYYFDYWGQGTLVTVSS | |
| 39 | EVQLVQSGGGLVQPGGSLRLSCAASGFTFSRNAMFWVRQAPGKGLE | SCB-VH-66 |
| | WVSGIGTGGATNYADSVKGRFTISRDNAKNSLYLQMNSLRAEDMA | |
| | VYYCARGRYYFDYWGQGTLVTVSS | |
| 40 | EVQLVQSGGGLVHPGGSLRLSCAGSGFTFSRNAMFWVRQAPGKGLE | SCB-VH-67 |
| | WVSGIGTGGATSYADSVKGRFTISRDNAKNSLYLQMNSLRAEDTAV | |
| | YYCARGRYYFDYWGQGTLVTVSS | |
| 41 | EVQLVQSGGGLVHPGGSLRLSCAGSGFTFSRNAMFWVRQAPGKGLE | SCB-VH-68 |
| | WVSGIGTGGATNYADSVKGRFTISRDNAKNSLYLQMNSLRAEDMA | |
| | VYYCARGRYYFPWWGQGTLVTVSS | |
| 42 | EVQLVESGGGLVHPGGSLRLSCAGSGFTFSRNAMFWVRQAPGKGLE | SCB-VH-69 |
| | WVSGIGTGGATNYADSVKGRFTISRDNAKNSLYLQMNSLRAEDMA | |
| | VYYCARGRYYFPWWGQGTLVTVSS | |
| 43 | EVQLVQSGGGLVQPGGSLRLSCAGSGFTFSRNAMFWVRQAPGKGLE | SCB-VH-70 |
| | WVSGIGTGGATNYADSVKGRFTISRDNAKNSLYLQMNSLRAEDMA | |
| | VYYCARGRYYFPWWGQGTLVTVSS | |
| 44 | EVQLVQSGGGLVHPGGSLRLSCAASGFTFSRNAMFWVRQAPGKGLE | SCB-VH-71 |
| | WVSGIGTGGATNYADSVKGRFTISRDNAKNSLYLQMNSLRAEDMA | |
| | VYYCARGRYYFPWWGQGTLVTVSS | |
| 45 | EVQLVQSGGGLVHPGGSLRLSCAGSGFTFSRNAMFWVRQAPGKGLE | SCB-VH-72 |
| | WVSGIGTGGATSYADSVKGRFTISRDNAKNSLYLQMNSLRAEDMA | |
| | VYYCARGRYYFPWWGQGTLVTVSS | |
| 46 | EVQLVQSGGGLVHPGGSLRLSCAGSGFTFSRNAMFWVRQAPGKGLE | SCB-VH-73 |
| | WVSGIGTGGATNYADSVKGRFTISRDNAKNSLYLQMNSLRAEDTAV | |
| | YYCARGRYYFPWWGQGTLVTVSS | |
| 47 | EVQLVQSGGGLVHPGRSLRLSCAGSGFTFSRNAMFWVRQAPGKGLE | SCB-VH-74 |
| | WVSGIGTGGATNYADSVKGRFTISRDNAKNSLYLQMNSLRAEDMA | |
| | VYYCARGRYYFDYWGQGTLVTVSS | |
| 48 | EVQLVQSGGGLVHPGGSLRLTCAGSGFTFSRNAMFWVRQAPGKGL | SCB-VH-75 |
| | EWVSGIGTGGATNYADSVKGRFTISRDNAKNSLYLQMNSLRAEDM | |
| | AVYYCARGRYYFDYWGQGTLVTVSS | |
| 49 | EVQLVQSGGGLVHPGGSLRLSCAGSGFTFSRNAMHWVRQAPGKGL | SCB-VH-76 |
| | EWVSGIGTGGATNYADSVKGRFTISRDNAKNSLYLQMNSLRAEDM | |
| | AVYYCARGRYYFDYWGQGTLVTVSS | |
| 50 | EVQLVQSGGGLVHPGGSLRLSCAGSGFTFSRNAMFWVRQAPGEGLE | SCB-VH-77 |
| | WVSGIGTGGATNYADSVKGRFTISRDNAKNSLYLQMNSLRAEDMA | |
| | VYYCARGRYYFDYWGQGTLVTVSS | |
| 51 | EVQLVQSGGGLVHPGGSLRLSCAGSGFTFSRNAMFWVRQAPGKGLE | SCB-VH-78 |
| | WVSGIGTGGATNYADSVKGRFTISRDEAKNSLYLQMNSLRAEDMA | |
| | VYYCARGRYYFDYWGQGTLVTVSS | |
| 52 | EVQLVQSGGGLVHPGGSLRLSCAGSGFTFSRNAMFWVRQAPGKGLE | SCB-VH-79 |
| | WVSGIGTGGATNYADSVKGRFTISRDNAKNSLYLQMNSLRAGDMA | |
| | VYYCARGRYYFDYWGQGTLVTVSS | |
| 53 | EVQLVQSGGGLVHPGGSLRLSCAGSGFTFDDYAMFWVRQAPGKGL | SCB-VH-80 |
| | EWVSGIGTGGATNYADSVKGRFTISRDNAKNSLYLQMNSLRAEDM | |
| | AVYYCARGRYYFDYWGQGTLVTVSS | |
| 54 | EVQLVQSGGGLVQPGGSLRLSCAASGFTFSRNAMFWVRQAPGKGLE | SCB-VH-81 |
| | WVSGIGTGGATSYADSVKGRFTISRDNAKNSLYLQMNSLRAEDTAV | |
| | YYCARGRYYFPWWGQGTLVTVSS | |
| 55 | EVQLVESGGGLVHPGGSLRLSCAASGFTFSRNAMFWVRQAPGKGLE | SCB-VH-82 |
| | WVSGIGTGGATSYADSVKGRFTISRDNAKNSLYLQMNSLRAEDTAV | |
| | YYCARGRYYFPWWGQGTLVTVSS | |
| 56 | EVQLVESGGGLVQPGGSLRLSCAGSGFTFSRNAMFWVRQAPGKGLE | SCB-VH-83 |
| | WVSGIGTGGATSYADSVKGRFTISRDNAKNSLYLQMNSLRAEDTAV | |
| | YYCARGRYYFPWWGQGTLVTVSS | |
| 57 | EVQLVESGGGLVQPGGSLRLSCAASGFTFSRNAMFWVRQAPGKGLE | SCB-VH-84 |
| | WVSGIGTGGATNYADSVKGRFTISRDNAKNSLYLQMNSLRAEDTAV | |
| | YYCARGRYYFPWWGQGTLVTVSS | |
| 58 | EVQLVESGGGLVQPGGSLRLSCAASGFTFSRNAMFWVRQAPGKGLE | SCB-VH-85 |
| | WVSGIGTGGATSYADSVKGRFTISRDNAKNSLYLQMNSLRAEDMA | |
| | VYYCARGRYYFPWWGQGTLVTVSS | |
| 59 | EVQLVQSGGGLVHPGGSLRLSCAASGFTFSRNAMFWVRQAPGKGLE | SCB-VH-86 |
| | WVSGIGTGGATSYADSVKGRFTISRDNAKNSLYLQMNSLRAEDTAV | |
| | YYCARGRYYFPWWGQGTLVTVSS | |
| 60 | EVQLVQSGGGLVQPGGSLRLSCAGSGFTFSRNAMFWVRQAPGKGLE | SCB-VH-87 |
| | WVSGIGTGGATSYADSVKGRFTISRDNAKNSLYLQMNSLRAEDTAV | |
| | YYCARGRYYFPWWGQGTLVTVSS | |
| 61 | EVQLVESGGGLVHPGGSLRLSCAGSGFTFSRNAMFWVRQAPGKGLE | SCB-VH-88 |
| | WVSGIGTGGATSYADSVKGRFTISRDNAKNSLYLQMNSLRAEDTAV | |
| | YYCARGRYYFPWWGQGTLVTVSS | |
| 62 | EVQLVQSGGGLVHPGGSLRLSCAGSGFTFSRNAMFWVRQAPGKGLE | SCB-VH-89 |
| | WVSGIGTGGATSYADSVKGRFTISRDNAKNSLYLQMNSLRAEDTAV | |
| | YYCARGRYYFPWWGQGTLVTVSS | |
| 63 | EVQLVESGGGLVQPGGSLRLSCAASGFTFSRNAMFWVRQAPGKGLE | SCB-VH-90 |
| | WVSGIGTGGATNYADSVKGRFTISRDNAKNSLYLQMNSLRAEDMA | |
| | VYYCARGRYYFPWWGQGTLVTVSS | |
| 64 | EVQLVESGGGLVQPGGSLRLSCAASGFTFSRNAMFWVRQAPGKGLE | SCB-VH-91 |
| | WVSGIGTGGATSYADSVKGRFTISRDNAKNSLYLQMNSLRAEDTAV | |
| | YYCARGRYYFDYWGQGTLVTVSS | |
| 65 | EVQLVQSGGGLVHPGGSLRLSCAGSGFTFSRNAMFWVRQAPGKGLE | SCB-VH-92 |
| | WVSGIGTGGATNYADSVKGRFTISRDNAKNSLYLQMNSLRAEDMA | |
| | VYYCARGRYYFDYWGQGTLVTVSS | |
| 66 | EVQLVESGGGLVQPGGSLRLSCAASGFTFSRNAMFWVRQAPGKGLE | SCB-VH-93 |
| | WVSGIGTGGATSYADSVKGRFTISRDNAKNSLYLQMNSLRAEDTAV | |
| | YYCARGRYYFPWWGQGTLVTVSS | |
| 67 | QVQLVQSGAEVKKPGASVKVSCKASGYAFTSYYMHWARQAPGQG | MEDI-1-VH |
| | LEWMGIINPSGGSTSYAQKFQGRVTMTRDTSTSTVYMELSSLRSEDT | |
| | AVYYCARGKWWLDYWGKGTLVTVSS | |
| 68 | QSVLTQPPSVSAAPGQKVTISCSGGSSNIGNSYVSWYQQLPGTAPKL | MEDI-1-VL |
| | LIYDNNKRPSGIPDRFSGSKSGTSATLAITGLQTGDEADYYCGTWDT | |
| | SLSANYVFGTGTKLTVL | |
| 69 | QVQLVQSGAEVKKPGASVKVSCKASGYAFTSYYMHWARQAPGQG | MEDI-2-VH |
| | LEWMGIINPSGGSTSYAQKFQGRVTMTRDTSTSTVYMELSSLRSEDT | |
| | AVYYCARGKWWLYNWGKGTLVTVSS | |
| 70 | QSVLTQPPSVSAAPGQKVTISCSGGSSNIGNSYVSWYQQLPGTAPKL | MEDI-2-VL |
| | LIYDNNKRPSGIPDRFSGSKSGTSATLAITGLQTGDEADYYCGTWDT | |
| | SQPPNPLFGTGTKLTVL | |
| 71 | QVQLVQSGAEVKKPGASVKVSCKASGYAFTSYYMHWARQAPGQG | MEDI-3-VH |
| | LEWMGIINPSGGSTSYAQKFQGRVTMTRDTSTSTVYMELSSLRSEDT | |
| | AVYYCARGKLLKNPWGKGTLVTVSS | |
| 72 | QSVLTQPPSVSAAPGQKVTISCSGGSSNIGNSYVSWYQQLPGTAPKL | MEDI-3-VL |
| | LIYDNNKRPSGIPDRFSGSKSGTSATLAITGLQTGDEADYYCGTWFG | |
| | TPASNYVFGTGTKLTVL | |
| 73 | QVQLVQSGAEVKKPGASVKVSCKASGYAFTSYYMHWARQAPGQG | MEDI-4-VH |
| | LEWMGIINPSGGSTSYAQKFQGRVIMTRDTSTSTVYMELSSLRSEDT | |
| | AVYYCARGKWWLYNWGKGTLVTVSS | |
| 74 | QSVLTQPPSVSAAPGQKVTISCSGGSSNIGNSYVSWYQQLPGTAPKL | MEDI-4-VL |
| | LIYDNNKRPSGIPDRFSGSKSGTSATLAITGLQTGDEADYYCGTWDT | |
| | SSPPQPIFGTGTKLTVL | |
| 75 | QVQLVQSGAEVKKPGASVKVSCKASGYAFTSYYMHWARQAPGQG | MEDI-5-VH |
| | LEWMGIINPSGGSTSYAQKFQGRVIMTRDTSTSTVYMELSSLRSEDT | |
| | AVYYCARGKWWLYDWGKGILVTVSS | |
| 76 | QSVLTQPPSVSAAPGQKVTISCSGGSSNIGNSYVSWYQQLPGTAPKL | MEDI-5-VL |
| | LIYDNNKRPSGIPDRFSGSKSGTSATLAITGLQTGDEADYYCGTWDT | |
| | SSPPQPIFGTGTKLTVL | |
| 77 | QVQLVQSGAEVKKPGASVKVSCKASGYAFTSYYMHWARQAPGQG | MEDI-6-VH |
| | LEWMGIINPSGGSTSYAQKFQGRVTMTRDTSTSTVYMELSSLRSEDT | |
| | AVYYCARGKYWMYDWGKGTLVTVSS | |
| 78 | QSVLTQPPSVSAAPGQKVTISCSGGSSNIGNSYVSWYQQLPGTAPKL | MEDI-6-VL |
| | LIYDNNKRPSGIPDRFSGSKSGTSATLAITGLQTGDEADYYCGTWDT | |
| | STTYHPIFGTGTKLTVL | |
| 79 | QVQLVQSGAEVKKPGASVKVSCKASGYAFTSYYMHWARQAPGQG | MEDI-7-VH |
| | LEWMGIINPSGGSTSYAQKFQGRVTMTRDTSTSTVYMELSSLRSEDT | |
| | AVYYCARGKWWWQYWGKGTLVTVSS | |
| 80 | QSVLTQPPSVSAAPGQKVTISCSGGSSNIGNSYVSWYQQLPGTAPKL | MEDI-7-VL |
| | LIYDNNKRPSGIPDRFSGSKSGTSATLAITGLQTGDEADYYCGTWDT | |
| | SSPPQPIFGTGTKLTVL | |
| 81 | QVQLVQSGAEVKKPGASVKVSCKASGYAFTSYYMHWARQAPGQG | MEDI-8-VH |
| | LEWMGIINPSGGSTSYAQKFQGRVTMTRDTSTSTVYMELSSLRSEDT | |
| | AVYYCARGKWWWQYWGKGTLVTVSS | |
| 82 | QSVLTQPPSVSAAPGQKVTISCSGGSSNIGNSYVSWYQQLPGTAPKL | MEDI-8-VL |
| | LIYDNNKRPSGIPDRFSGSKSGTSATLAITGLQTGDEADYYCGTWDT | |
| | STTYHPIFGTGTKLTVL | |
| 83 | QVQLVQSGAEVKKPGASVKVSCKASGYAFTSYYMHWARQAPGQG | MEDI-9-VH |
| | LEWMGIINPSGGSTSYAQKFQGRVTMTRDTSTSTVYMELSSLRSEDT | |
| | AVYYCARGKWWLYNWGKGTLVTVSS | |
| 84 | QSVLTQPPSVSAAPGQKVTISCSGGSSNIGNSYVSWYQQLPGTAPKL | MEDI-9-VL |
| | LIYDNNKRPSGIPDRFSGSKSGTSATLAITGLQTGDEADYYCGTWDT | |
| | STTMYPLFGTGTKLTVL | |
| 85 | QVQLVQSGAEVKKPGASVKVSCKASGYAFTSYYMHWARQAPGQG | MEDI-10-VH |
| | LEWMGIINPSGGSTSYAQKFQGRVTMTRDTSTSTVYMELSSLRSEDT | |
| | AVYYCARGKWWLYDWGKGILVTVSS | |
| 86 | QSVLTQPPSVSAAPGQKVTISCSGGSSNIGNSYVSWYQQLPGTAPKL | MEDI-10-VL |
| | LIYDNNKRPSGIPDRFSGSKSGTSAILAITGLQTGDEADYYCGTWDT | |
| | STVLTPIFGTGTKLTVL | |
| 87 | QVQLVQSGAEVKKPGASVKVSCKASGYAFTSYYMHWARQAPGQG | MEDI-11-VH |
| | LEWMGIINPSGGSTSYAQKFQGRVTMTRDTSTSTVYMELSSLRSEDT | |
| | AVYYCARGKWWFYDWGKGTLVTVSS | |
| 88 | QSVLTQPPSVSAAPGQKVTISCSGGSSNIGNSYVSWYQQLPGTAPKL | MEDI-11-VL |
| | LIYDNNKRPSGIPDRFSGSKSGTSATLAITGLQTGDEADYYCGTWDT | |
| | SPSMIPLFGTGTKLTVL | |
| 89 | QVQLVQSGAEVKKPGASVKVSCKASGYAFTSYYMHWARQAPGQG | MEDI-12-VH |
| | LEWMGIINPSGGSTSYAQKFQGRVTMTRDTSTSTVYMELSSLRSEDT | |
| | AVYYCARGKWWFYDWGKGTLVTVSS | |
| 90 | QSVLTQPPSVSAAPGQKVTISCSGGSSNIGNSYVSWYQQLPGTAPKL | MEDI-12-VL |
| | LIYDNNKRPSGIPDRFSGSKSGTSATLAITGLQTGDEADYYCGTWDT | |
| | STTMYPLFGTGTKLTVL | |
| 91 | QVQLVQSGAEVKKPGASVKVSCKASGYAFTSYYMHWARQAPGQG | MEDI-13-VH |
| | LEWMGIINPSGGSTSYAQKFQGRVTMTRDTSTSTVYMELSSLRSEDT | |
| | AVYYCARGKWWLYDWGKGTLVTVSS | |
| 92 | QSVLTQPPSVSAAPGQKVTISCSGGSSNIGNSYVSWYQQLPGTAPKL | MEDI-13-VL |
| | LIYDNNKRPSGIPDRFSGSKSGTSATLAITGLQTGDEADYYCGTWDT | |
| | STTLQPLFGTGTKLTVL | |
| 93 | QVQLVQSGAEVKKPGASVKVSCKASGYAFTSYYMHWARQAPGQG | MEDI-14-VH |
| | LEWMGIINPSGGSTSYAQKFQGRVTMTRDTSTSTVYMELSSLRSEDT | |
| | AVYYCARGKWWLYNWGKGTLVTVSS | |
| 94 | QSVLTQPPSVSAAPGQKVTISCSGGSSNIGNSYVSWYQQLPGTAPKL | MEDI-14-VL |
| | LIYDNNKRPSGIPDRFSGSKSGTSATLAITGLQTGDEADYYCGTWDT | |
| | SPPTKPLFGTGTKLTVL | |
| 95 | QVQLVQSGAEVKKPGASVKVSCKASGYAFTSYYMHWARQAPGQG | MEDI-15-VH |
| | LEWMGIINPSGGSTSYAQKFQGRVTMTRDTSTSTVYMELSSLRSEDT | |
| | AVYYCARGKWWLYNWGKGTLVTVSS | |
| 96 | QSVLTQPPSVSAAPGQKVTISCSGGSSNIGNSYVSWYQQLPGTAPKL | MEDI-15-VL |
| | LIYDNNKRPSGIPDRFSGSKSGTSATLAITGLQTGDEADYYCGTWDT | |
| | STHRHPLFGTGTKLTVL | |
| 97 | QVQLVQSGAEVKKPGASVKVSCKASGYAFTSYYMHWARQAPGQG | MEDI-16-VH |
| | LEWMGIINPSGGSTSYAQKFQGRVTMTRDTSTSTVYMELSSLRSEDT | |
| | AVYYCARGKWWLYNWGKGTLVTVSS | |
| 98 | QSVLTQPPSVSAAPGQKVTISCSGGSSNIGNSYVSWYQQLPGTAPKL | MEDI-16-VL |
| | LIYDNNKRPSGIPDRFSGSKSGTSATLAITGLQTGDEADYYCGTWDT | |
| | STTYHPIFGTGTKLTVL | |
| 99 | QVQLVQSGAEVKKPGASVKVSCKASGYAFTSYYMHWARQAPGQG | MEDI-17-VH |
| | LEWMGIINPSGGSTSYAQKFQGRVTMTRDTSTSTVYMELSSLRSEDT | |
| | AVYYCARGKWWWQHWGKGTLVTVSS | |
| 100 | QSVLTQPPSVSAAPGQKVTISCSGGSSNIGNSYVSWYQQLPGTAPKL | MEDI-17-VL |
| | LIYDNNKRPSGIPDRFSGSKSGTSATLAITGLQTGDEADYYCGTWDT | |
| | SPVDRPIFGTGTKLTVL | |
| 101 | QVQLVQSGAEVKKPGASVKVSCKASGYAFTSYYMHWARQAPGQG | MEDI-18-VH |
| | LEWMGIINPSGGSTSYAQKFQGRVTMTRDTSTSTVYMELSSLRSEDT | |
| | AVYYCARGKWWWQHWGKGTLVTVSS | |
| 102 | QSVLTQPPSVSAAPGQKVTISCSGGSSNIGNSYVSWYQQLPGTAPKL | MEDI-18-VL |
| | LIYDNNKRPSGIPDRFSGSKSGTSATLAITGLQTGDEADYYCGTWDT | |
| | STTPMPVFGTGTKLTVL | |
| 103 | QVQLVQSGAEVKKPGASVKVSCKASGYAFTSYYMHWARQAPGQG | MEDI-19-VH |
| | LEWMGIINPSGGSTSYAQKFQGRVTMTRDTSTSTVYMELSSLRSEDT | |
| | AVYYCARGKWWWQHWGKGTLVTVSS | |
| 104 | QSVLTQPPSVSAAPGQKVTISCSGGSSNIGNSYVSWYQQLPGTAPKL | MEDI-19-VL |
| | LIYDNNKRPSGIPDRFSGSKSGTSATLAITGLQTGDEADYYCGTWDT | |
| | STTYHPIFGTGTKLTVL | |
| 105 | QVQLVQSGAEVKKPGASVKVSCKASGYAFTSYYMHWARQAPGQG | MEDI-20-VH |
| | LEWMGIINPSGGSTSYAQKFQGRVTMTRDTSTSTVYMELSSLRSEDT | |
| | AVYYCARGKYWMYDWGKGTLVTVSS | |
| 106 | QSVLTQPPSVSAAPGQKVTISCSGGSSNIGNSYVSWYQQLPGTAPKL | MEDI-20-VL |
| | LIYDNNKRPSGIPDRFSGSKSGTSATLAITGLQTGDEADYYCGTWDT | |
| | STVWEWPFGTGTKLTVL | |
| 107 | QVQLVQSGAEVKKPGASVKVSCKASGYAFTSYYMHWARQAPGQG | MEDI-21-VH |
| | LEWMGIINPSGGSASYAQKFQGRVTMTRDTSTSTVYMELSSLRSEDT | |
| | AVYYCARGKYWMYDWGKGTLVTVSS | |
| 108 | QSVLTQPPSVSAAPGQKVTISCSGGSSNIGNSYVSWYQQLPGTAPKL | MEDI-21-VL |
| | LIYDNNKRPSGIPDRFSGSKSGTSATLAITGLQTGDEAVYFCGTWDTS | |
| | TVWEWPFGTGTKLTVL | |
| 109 | QVQLVQSGAEVKKPGASVKVSCKASGYAFTSYYMHWARQAPGQG | MEDI-22-VH |
| | LEWMGIINPSGGSTSYAQKFQGRVTMTRDTSTSTVYMELSSLRSEDT | |
| | AVYYCARGKYWMYDWGKGTLVTVSS | |
| 110 | QPVLTQPPSVSAAPGQKVTISCSGGSSNIGNSYVSWYQQLPGTAPKL | MEDI-22-VL |
| | LIYDNNKRPSGIPDRFSGSKSGTSATLAITGLQTGDEADYFCGTWDTS | |
| | TVWEWPFGTGTKLTVL | |
| 111 | QVQLVQSGAEVRKPGASVKVSCKASGYAFTSYYMHWARQAPGQG | MEDI-23-VH |
| | LEWMGIINPSGGSTSYAQKFQGRVTMTRDTSTSTVYMELSSLRSEDT | |
| | AVYYCARGKYWMYDWGKGTLVTVSS | |
| 112 | QSVLTQPPSVSAAPGQKVTISCSGGSSNIGNNYVSWYQQLPGTAPKL | MEDI-23-VL |
| | LIYDNNKRPPGIPDRFSGSKSGTSATLAITGLQTGDEADYYCGTWDT | |
| | STVWEWPFGTGTKLTVL | |
| 113 | QVQLVQSGAEVKKPGASVKVSCKASGYAFTSYYMHWARQAPGQG | MEDI-24-VH |
| | LEWMGIINPRGGSTSYAQKFQGRVTMTRDTSTSTVYMELSSLRSEDT | |
| | AVYYCARGKYWMYDWGKGTLVTVSS | |
| 114 | QSVLTQPPSVSAAPGQKVTISCSGGSSNIGNSYVSWYQQLPGTAPKL | MEDI-24-VL |
| | LIYDNNKRPSGIPDRFSGSKSGTSATLAITGLQTGDEADYFCGTWDTS | |
| | TVWEWPFGTGTKLTVL | |
| 115 | QVQLVQSGAEVKKPGASVKVSCKASGYAFTSYYMHWARQAPGQG | MEDI-25-VH |
| | LEWMGIINPRGGSASYAQKFQGRVSMTRDTSTSTVYMELSSLRSEDT | |
| | AVYYCARGKYWMYDWGKGTLVTVSS | |
| 116 | QSVLTQPPSVSAAPGQKVTISCSGGSSNIGNSYVSWYQQLPGTAPKL | MEDI-25-VL |
| | LIYDNNKRPSGIPDRFSGSKSGTTATLAITGLQTGDEADYYCGTWVT | |
| | STVWEWPFGTGTKLTVL | |
| 117 | QVQLVQSGAEVKKPGASVKVSCKASGYAFTSYYMHWARQAPGQG | MEDI-26-VH |
| | LEWMGIINPSGGSTSYAQKFQGRVTMTRDTSTSTVYMELSSLRSEDT | |
| | AVYYCARGKYWMYDWGKGTLVTVSS | |
| 118 | QSVLTQPPSVSAAPGQKVTISCSGGSSNIGNSYVSWYQQLPGTAPKL | MEDI-26-VL |
| | LIYDNNKRPSGIPDRFSGSKSGTSATLAITGLQTGDEADYFCGTWDTS | |
| | TVWEWPFGTGTKLTVL | |
| 119 | QVQLVQSGAEVRKPGASVKVSCKASGYAFTSYYMHWARQAPGQG | MEDI-27-VH |
| | LEWMGIINPSGGSTSYAQKFQGRVTMTRDTSTSTVYMELSSLRPEDT | |
| | AVYYCARGKYWMYDWGKGTQVTVSS | |
| 120 | QSVLTQPPLVSAAPGQKVTISCSGGSSNIGNSYVSWYQRLPGTAPKL | MEDI-27-VL |
| | LIYDNNKRPSGIPDRFSGSKSGTSATLAITGLQTGDEADYYCGTWDT | |
| | STVWEWPFGTGTKLTVL | |
| 121 | QVQLVQSGAEVKKPGASVKVSCKASGYAFTSYYMHWARQAPGQG | MEDI-28-VH |
| | LEWMGIINPSGGSTSYAQKFQGRVTMTRDTSTSTVYMELSSLRSEDT | |
| | AVYYCARGKYWMYDWGNGTLVTVSS | |
| 122 | LPVLTQPPSVSAAPGQKVTISCSGGSSSIGNSYVSWYQQLPGAAPKLL | MEDI-28-VL |
| | IYDNNKRPSGIPDRFSGFRSGTSATLAITGLQTGDEADYYCGTWDTSP | |
| | VWEWPFGTGTKLTVL | |
| 123 | QVQLVQSGAEVKKPGASVKVSCKASGYAFTSYYMHWARQAPGQG | MEDI-29-VH |
| | LEWMGIINPSGGSTSYAQKFQGRVTMTRDTSTSTVYMELSSLRSEDT | |
| | AVYYCARGKYWMYDWGKGTRVTVSS | |
| 124 | QSVLTQPPSVSAAPGQKVTISCSGGSSNIGNSYVSWYQQLPGTAPKL | MEDI-29-VL |
| | LIYDNNKRPSGIPDRFSGSKSGTSATLAITGLQTGDEADYYCGTWDT | |
| | SPVWEWPFGTGTKLTVL | |
| 125 | QVQLVQSGAEVKKPGASVKVSCKASGYAFTSYYMHWARQAPGQG | MEDI-30-VH |
| | LEWMGIINPSGGSTSYAQKFQGRVTMTRDTSTSTVYMELSSLRSEDT | |
| | AVYYCARGKYWMYDWGKGTLVTVSS | |
| 126 | QSVLTQPPSVSAAPGQKVTISCSGGSSNIGNSYVSWYQRLPGAAPKL | MEDI-30-VL |
| | LIYDNNKRPSGIPDRFSGSKSGTSATLAITGLQTGDEADYYCGTWDT | |
| | STVWEWPFGTGTKLTVL | |
| 127 | QVQLVQSGAEVKKPGASVKVSCKASGYAFTSYYMHWARQAPGQG | MEDI-31-VH |
| | LEWMGIINPSGGSTSYAQKFQGRVTMTRDTSTSTVYMELSSLRSEDT | |
| | AVYYCARGKYWMYDWGKGTLVTVSS | |
| 128 | QSVLTQPPSVSAAPGQKVTISCSGGSSSIGNSYVSWYQQLPGTAPKLL | MEDI-31-VL |
| | IYDNNKRPSGIPDRFSGSKSGTSATLAITGLQTGDEADYYCGTWATS | |
| | PVWEWPFGTGTKLTVL | |
| 129 | QVQLVQSGAEVKKPGASVKVSCKASGYAFTSYYMHWARQAPGQG | MEDI-32-VH |
| | LEWMGIINPSGGSTSYAQKFQGRVTMTRDTSTSTVYMELSSLRSEDT | |
| | AVYYCARGKYWMYDWGKGTLVTVSS | |
| 130 | QSVLTQPPSVSAAPGQKVTISCSGGSSNIGNSYVSWYQQLPGTAPKL | MEDI-32-VL |
| | LIYDNNKRPSGIPDRFSGSKSGTSATLAITGLQTGDEADYFCGTWDTS | |
| | TAWEWPFGTGTKLTVL | |
| 131 | QVQLVQSGAEEKKPGASVKVSCKASGYAFTSYYMHWARQAPGQG | MEDI-33-VH |
| | LEWMGIINPSGGSTSYAQKFQGRVTMTRDTSTSTVYMELSSLRSEDT | |
| | AVYYCARGKYWMYDWGKGTLVTVSS | |
| 132 | QSALTQPPSVSAAPGQKVTISCSGGSSNIGNSYVSWYQQLPGTAPKL | MEDI-33-VL |
| | LIYDNNKRPSGIPDRFSGSKSGTSATLAITGLQTGDEADYFCGTWDTS | |
| | TVWEWPFGTGTKLTVL | |
| 133 | QVQLVQSGAEVKKPGASVKVSCKASGYAFTSYYMHWARQAPGQG | MEDI-34-VH |
| | LEWMGIINPSGGSTSYAQKFQGRVSMTRDTSTSTVYMELSSLRSEDT | |
| | AVYYCARGKYWMYDWGKGTLVTVSS | |
| 134 | QSVLTQPPSVSAAPGQKVTISCSGGSSNIGNSYVSWYQQLPGTAPKL | MEDI-34-VL |
| | LIYDNNKRPSGIPDRFSGSKSGTSATLAITGLQTGDEADYFCGTWDTS | |
| | TVWEWPFGTGTKLTVL | |
| 135 | QVQLVQSGAEVKKPGASVKVSCKASGYAFTSYYMHWARQAPGQG | MEDI-35-VH |
| | LEWMGIINPSGGSTSYAQKFQGRVTMTRDTSTSTVYMELSSLRSEDT | |
| | AVYYCARGKYWMYDWGKGTLVTVSS | |
| 136 | QSVLTQPPSVSAAPGQKVTISCSGGSSNIGNSYVSWYQQLPGTAPKL | MEDI-35-VL |
| | LIYDNNKRPSGIPDRFSGSKSGTSATLAITGLQTGDEADYYCGTWDT | |
| | SPVWEWPFGTGTKLTVL | |
| 137 | QVQLVQSGAEVKKPGASVKVSCKASGYAFTSYYMHWARQAPGQG | MEDI-36-VH |
| | LEWMGIINPSGGSASYAQKFQGRVTMTRDTSTSTVYMELSSLRSEDT | |
| | AVYYCARGKYWMYDWGKGTLVTVSS | |
| 138 | QSVLTQPPSVSAAPGQKVTISCSGGSSNIGNSYVSWYQQLPGTAPKL | MEDI-36-VL |
| | LIYDNNKRPSGIPDRFSGSKSGTSATLAITGLQTGDEADYYCGTWDS | |
| | STVWEWPFGTGTKLTVL | |
| 139 | QVQLVQSGAEVKKPGASVKVSCKASGYAFTSYYMHWARQAPGQG | MEDI-37-VH |
| | LEWMGIINPRGGSTSYAQKFQGRVAMTRDTSTSTVYMELSSLRPEDT | |
| | AVYYCARGKYWMYDWGKGTLVTVSS | |
| 140 | QSVLTQPPSVSAAPGQKVTISCSGGGSSIGNSYVSWYQQLPGTAPKL | MEDI-37-VL |
| | LIYDNNKRPSGVPDRFSGSKSGTSATLAITGLQTGDEADYYCGTWDT | |
| | SPVWEWPFGTGTKLTVL | |
| 141 | QVQLVQSGAEVKKPGASVKVSCKASGYAFTSYYMHWARQAPGQG | MEDI-38-VH |
| | LEWMGIINPSGGSASYAQKFQGRVTMTRDTSTSTVYMELSSLRSEDT | |
| | AVYYCARGKYWMYDWGKGTLVTVSS | |
| 142 | QSVLTQPPSVSAAPGQKVTISCSGGSSNIGNSYVSWYQQLPGTAPKL | MEDI-38-VL |
| | LIYDNNKRPSGIPDRFSGSKSGTSATLAITGLQTGDEADYFCGTWDTS | |
| | TVWEWPFGTGTKLTVL | |
| 143 | QVQLVQSGAEVKKPGASVKVSCKASGYAFTSYYMHWARQAPGQG | MEDI-39-VH |
| | LEWMGIINPRGGSTSYAQKFQGRVTMTRDTSTSTVYMELSSLRSEDT | |
| | AVYYCARGKYWMYDWGKGTLVTVSS | |
| 144 | QSVLTQPPSVSAAPGQKVTISCSGGSSNIGNSYVSWYQQLPGTAPKL | MEDI-39-VL |
| | LIYDNNKRPSGIPDRFSGSKSGTSATLAITGLQTGDEADYYCGTWDT | |
| | STAWEWPFGTGTKLTVL | |
| 145 | QVQLVQSGAEVKKPGASVKVSCKASGYAFTSYYMHWARQAPGQG | MEDI-40-VH |
| | LEWMGIINPSGGSTSYAQKFQGRVTMTRDTSTSTVYMELSSLRSEDT | |
| | AVYYCARGKYWMYDWGKGTLVTVSS | |
| 146 | QSVLTQPPSVSAAPGQKVTISCSGGSSNIGNSYVSWYQQLPGTAPKL | MEDI-40-VL |
| | LIYDNNKRPSGIPDRFSGSKSGTSATLAITGLQTGDEADYYCGTWDS | |
| | STVWEWPFGTGTKLTVL | |
| 147 | QVQLVQSGAEVRKPGASVKVSCKASGYAFTSYYMHWARQAPGQG | MEDI-41-VH |
| | LEWMGIINPSGGSTSYAQKFQGRVTMTRDTSTSTVYMELSSLRPEDT | |
| | AVYYCARGKYWMYDWGKGTLVTVSG | |
| 148 | QSVLTQPPSVSAAPGQKVTISCSGGSTNIGNSYVSWYQRLPGTAPKL | MEDI-41-VL |
| | LIYDNNKRPPGIPDRFSGSKSGTSATLAITGLQTGDEADYYCGTWDT | |
| | STVWEWPFGTGTKLTVL | |
| 149 | QVQLVQSGAEVKKPGASVKVSCKASGYAFTSYYMHWARQAPGQG | MEDI-42-VH |
| | LEWVGIINPSGGSTSYAQKFQGRVTMTRDTSTSTVYMELSSLRSGDT | |
| | AVYYCARGKYWMYDWGKGTLVTVSS | |
| 150 | QAVLTQPPSVSAAPGQKVTISCSGGSSNIGNSYVSWYQRLPGAAPKL | MEDI-42-VL |
| | LIYDNNKRPSGIPDRFSGSKSGTSATLAITGLQTGDEADYYCGTWDT | |
| | STGWEWPFGTGTKLTVL | |
| 151 | QVQLVQSGAEVKKPGASVKVSCKASGYAFTSYYMHWVRQAPGQG | MEDI-37GL-VH |
| | LEWMGIINPRGGSTSYAQKFQGRVTMTRDTSTSTVYMELSSLRSEDT | |
| | AVYYCARGKYWMYDWGKGTLVTVSS | |
| 152 | QSVLTQPPSVSAAPGQKVTISCSGGGSSIGNSYVSWYQQLPGTAPKL | MEDI-37GL-VL |
| | LIYDNNKRPSGIPDRFSGSKSGTSATLGITGLQTGDEADYYCGTWDT | |
| | SPVWEWPFGTGTKLTVL | |
| 153 | EVQLLESGGGLVQPGGSLRLSCAVSGFTFSNYAMSWVRQAPGKGLE | AJOU-1-VH |
| | WVSAISSGGGNIYYADSVKGRFTISRDNSKNTLYLQMNSLRAEDTA | |
| | VYYCAKLRRYFDYWGQGTLVTVSS | |
| 154 | EVQLLESGGGLVQPGGSLRLSCAASGFTFSDYAMSWVRQAPGKGLE | AJOU-2-VH |
| | WVSAISSGGSSIYYADSVKGRFTISRDNSKNTLHLQMNSLRAEDTAV | |
| | YYCARGPQRSATAVFDYWGQGTLVTVSS | |
| 155 | EVQLLESGGGLVQPGGSLRLSCAASGFTFSNYAMSWVRQAPGKGLE | AJOU-3-VH |
| | WVSWISPNSGNIYYADSVKGRFTISRDNSKNTLYLQMNSLRAEDTA | |
| | VYYCARRPLSAAWSHSSYYNAMDVWGQGTLVTVSS | |
| 156 | EVQLLESGGGLVQPGGSLRLSCAASGFTFSGYAMSWVRQAPGKGLE | AJOU-4-VH |
| | WVSLISHSGSNTYYADSVKGRFTISRDNSKNTLYLQMNSLRAEDTA | |
| | VYYCARPHRAFDYWGQGTLVTVSS | |
| 157 | EVQLLESGGGLVQPGGSLRLSCAASGFTFSNYAMSWVRQAPGKGLE | AJOU-5-VH |
| | WVSGISHGSGSIYYADSVKGRFTISRDNSKNTLYLQMNSLRAEDTAV | |
| | YYCARPHRAFDYWGQGTLVTVSS | |
| 158 | EVQLLESGGGLVQPGGSLRLSCAASGFTFSNYAMSWVRQAPGKGLE | AJOU-6-VH |
| | WVSGISHGNGSIYYADSVKGRFTISRDNSKNTLYLQMNSLRAEDTA | |
| | VYYCAKTGRHFDYWGQGTLVTVSS | |
| 159 | EVQLLESGGGLVQPGGSLRLSCAASGFTFSNYAMSWVRQAPGKGLE | AJOU-7-VH |
| | WVSSISPSGSSIYYADSVKGRFTISRDNSKNTLYLQMNSLRAEDTAV | |
| | YYCARSYRAFDYWGQGTLVTVSS | |
| 160 | EVQLLESGGGLVQPGGSLRLSCAASGFTFSNYAMSWVRQAPGKGLE | AJOU-8-VH |
| | WVSAISPSGGSIYYADSVKGRFTISRDNSKNILYLQMNSLRAEDTAV | |
| | YYCARAKRAFDYWGQGTLVTVSS | |
| 161 | EVQLLESGGGLVQPGGSLRLSCAASGFTFSNYAMSWVRQAPGKGLE | AJOU-9-VH |
| | WVSAISPGSGSTYYADSVKGRFTISRDNSKNTLYLQMNSLRAEDTAV | |
| | YYCAKFRRHFDYWGQGTLVTVSS | |
| 162 | EVQLLESGGGLVQPGGSLRLSCAASGFTFSNYAMSWVRQAPGKGLE | AJOU-10-VH |
| | WVSAISSGGGNIYYADSVKGRFTISRDNSKNTLYLQMNSLRAEDTA | |
| | VYYCARVHRAFDYWGQGTLVTVSS | |
| 163 | EVQLLESGGGLVQPGGSLRLSCAASGFTFSNYAMSWVRQAPGKGLE | AJOU-69-VH |
| | WVSAITSSGRSIYYADSVKGRFTISRDNSKNTLYLQMNSLRAEDTAV | |
| | YYCARVHRAFDYWGQGTLVTVSs | |
| 164 | EVQLLESGGGLVQPGGSLRLSCAASGFTFSNYAMSWVRQAPGKGLE | AJOU-70-VH |
| | WVSAITSSGANIYYADSVKGRFTISRDNSKNTLYLQMNSLRAEDTAV | |
| | YYCARVHRAFDYWGQGTLVTVSS | |
| 165 | EVQLLESGGGLVQPGGSLRLSCAASGFTFSNYAMSWVRQAPGKGLE | AJOU-71-VH |
| | WVSAITSSGGNIYYADSVKGRFTISRDNSKNTLYLQMNSLRAEDTAV | |
| | YYCARVHRAFDYWGQGTLVTVSS | |
| 166 | EVQLLESGGGLVQPGGSLRLSCAASGFTFSNYAMSWVRQAPGKGLE | AJOU-72-VH |
| | WVSAITAGGGSIYYADSVKGRFTISRDNSKNTLYLQMNSLRAEDTA | |
| | VYYCARVHRAFDYWGQGTLVTVSS | |
| 167 | EVQLLESGGGLVQPGGSLRLSCAASGFTFSRHAMAWVRQAPGKGLE | AJOU-83-VH |
| | WVSAITSSGRSIYYADSVKGRFTISRDNSKNTLYLQMNSLRAEDTAV | |
| | YYCARVHRAFDYWGQGTLVTVSS | |
| 168 | QSVLTQPPSASGTPGQRVTISCSGSSSNIGNNYVNWYQQLPGTAPKL | AJOU-33-VL |
| | LIYDNSHRPSGVPDRFSGSKSGTSASLAISGLRSEDEADYYCGTWDA | |
| | SLSAYVFGGGTKLTVL | |
| 169 | QSVLTQPPSASGTPGQRVTISCSGSSSNIGNNNVSWYQQLPGTAPKLL | AJOU-34-VL |
| | IYANSKRPSGVPDRFSGSKSGTSASLAISGLRSEDEADYYCGSWDDS | |
| | LSAYVFGGGTKLTVL | |
| 170 | QSVLTQPPSAPGTPGQRVTISCTGSSSNIGSNSVNWYQQLPGTAPKLL | AJOU-35-VL |
| | IYDDSHRPSGVPDRFSGSKSGTSASLAISGLRSEDEADYYCDAWDSS | |
| | LSAYVFGGGTKLTVL | |
| 171 | QSVLTQPPSASGTPGQRVTLSCTGSSSNIGSNYVSWYQQLPGTAPKL | AJOU-36-VL |
| | LIYADSQRPSGVPDRFSGSKSGTSASLAISGLRSEDEADYYCGTWDD | |
| | SLSGYVFGGGTKLTVL | |
| 172 | QSVLTQPPSASGTPGQRVTISCSSSSSNIGSNYVSWYQQLPGTAPKLLI | AJOU-37-VL |
| | YSDSHRPSGVPDRFSGSKSGTSASLAISGLRSEDEADYYCGSWDYSL | |
| | SAYVFGGGTKLTVL | |
| 173 | QSVLTQPPSASGTPGQRVTISCTGSSSNIGNNTVSWYQQLPGTAPKLL | AJOU-38-VL |
| | IYDNSHRPSGVPDRFSGSKSGTSASLAISGLQSEDEADYYCGSWDYS | |
| | LSAYVFGGGTKLTVL | |
| 174 | QSVLTQPPSASGTPGQRVTISCTGSSSNIGNNDVNWYQQLPGTAPKL | AJOU-39-VL |
| | LIYYDSQRPSGVPDRFSGSKSGTSASLAISGLRSEDEADYYCATWDA | |
| | SLSAYVFGGGTKLTVL | |
| 175 | QSVLTQPPSASGTPGQRVTISCSGSSSNIGSNAVNWYQQLPGTAPKLL | AJOU-40-VL |
| | IYYDNQRPSGVPDRFSGSKSGTSASLAISGLRSEDEADYYCGTWDDS | |
| | LNGYVFGGGTKLTVL | |
| 176 | QSVLTQPPSASGTPGQRVTISCSGSSSNIGNNAVTWYQQLPGTAPKLL | AJOU-41-VL |
| | IYDDSHRPSGVPDRFSGSKSGTSASLAISGLRSEDEADYYCGSWDYS | |
| | LSAYVFGGGTKLTVL | |
| 177 | QSVLTQPPSASGTPGQRVTISCSGSSSNIGSNTFNWYQQLPGTAPKLLI | AJOU-42-VL |
| | YADSHRPSGVPDRFSGSKSGTSASLAISGLRSEDEADYYCGTWDYSL | |
| | SGYVLGGGTKLTVL | |
| 178 | QSVLTQPPSASGTPGQRVTISCSGSSSNIGSNTFNWYQQLPGTAPKLLI | AJOU-77-VL |
| | YADSHRPSGVPDRFSGSKSGTSASLAISGLRSEDEADYYCGTWDYSL | |
| | SGYVLGGGTKLTVL | |
| 179 | QSVLTQPPSASGTPGQRVTISCSGSSSNIGSNTFNWYQQLPGTAPKLLI | AJOU-78-VL |
| | YADSHRPSGVPDRFSGSKSGTSASLAISGLRSEDEADYYCGTWDYSL | |
| | RGYVLGGGTKLTVL | |
| 180 | QSVLTQPPSASGTPGQRVTISCSGSSSNIGSNTFNWYQQLPGTAPKLLI | AJOU-79-VL |
| | YADSHRPSGVPDRFSGSKSGTSASLAISGLRSEDEADYYCGYWDYSL | |
| | SGYVLGGGTKLTVL | |
| 181 | QSVLTQPPSASGTPGQRVTISCSGSSSNIGSNTFNWYQQLPGTAPKLLI | AJOU-80-VL |
| | YADSHRPSGVPDRFSGSKSGTSASLAISGLRSEDEADYYCGTWDYSL | |
| | SGYVLGGGTKLTVL | |
| 182 | QSVLTQPPSASGTPGQRVTISCSGSSANSRTDGFNWYQQLPGTAPKL | AJOU-86-VL |
| | LIYADSHRPSGVPDRFSGSKSGTSASLAISGLRSEDEADYYCGTWDY | |
| | SLSGYVLGGGTKLTVLG | |
| 183 | QSVLTQPPSASGTPGQRVTISCSGSAQFGSRDNFNWYQQLPGTAPKL | AJOU-87-VL |
| | LIYADSHRPSGVPDRFSGSKSGTSASLAISGLRSEDEADYYCGTWDY | |
| | SLSGYVLGGGTKLTVLG | |
| 184 | QSVLTQPPSASGTPGQRVTISCSGSTKQMHNYQFNWYQQLPGTAPK | AJOU-88-VL |
| | LLIYADSHRPSGVPDRFSGSKSGTSASLAISGLRSEDEADYYCGTWD | |
| | YSLSGYVLGGGTKLTVLG | |
| 185 | QSVLTQPPSASGTPGQRVTISCSGSLLRGENLQFNWYQQLPGTAPKL | AJOU-89-VL |
| | LIYADSHRPSGVPDRFSGSKSGTSASLAISGLRSEDEADYYCGTWDY | |
| | SLSGYVLGGGTKLTVLG | |
| 186 | QSVLTQPPSASGTPGQRVTISCSGSPLFPDSGSFNWYQQLPGTAPKLL | AJOU-90-VL |
| | IYADSHRPSGVPDRFSGSKSGTSASLAISGLRSEDEADYYCGTWDYS | |
| | LSGYVLGGGTKLTVLG | |
| 187 | QSVLTQPPSASGTPGQRVTISCSGSAALDLSPSFNWYQQLPGTAPKLL | AJOU-91-VL |
| | IYADSHRPSGVPDRFSGSKSGTSASLAISGLRSEDEADYYCGTWDYS | |
| | LSGYVLGGGTKLTVLG | |
| 188 | QVQLVQSGAEVKKPGASVKVSCKASGYTFTNYGISWVRQAPGQGL | REGN-VH-3 |
| | EWMGWISVYNGKTNYAQKLQGRVTMTTDTSTTTAYMEMRSLRSD | |
| | DTAVYYCARGSGYDLDYWGQGTLVSVSS | |
| 189 | EVQLVESGGGLVQPGGSLRLSCAASGFTFSSFWMTWVRQAPGKGLE | REGN-VH-19 |
| | WVANIKQDGSEKYYVDSVKGRFTISRDNAKNSLYLQMNSLRAEDT | |
| | AVYYCARDPGRTMVRGGIRYYYGMDVWGQGTTVTVSS | |
| 190 | EVKLAESGGGLVQPGGSLRLSCAASGFTFSSHWMNWVRQAPGKGL | REGN-VH-3 5 |
| | EWVANIKQDGSDKYYVDSVKGRFTISRDNAKNSLYLQLNSLIAEDT | |
| | AVYYCARDRGVRPPRGAFDIWGQGTMVTVSS | |
| 191 | QVQLVQSGAEVKKPGASVKVSCKASGYTFNSYGISWVRQAPGQGL | REGN-VH-51 |
| | EWMGWIRTYNGNTNYAQKLQGRVTMTTDTSTSTAYMELRSLRSDD | |
| | TAVYYCARDEARIVVAGTTPYYYGMDVWGQGTTVTVSS | |
| 192 | QVQLVESGGGLVQPGGSLRLSCAVSGFTISDHYMSWIRQAPGKGLE | REGN-VH-67 |
| | WISYISSSGSKIYYADSVKGRFTISRDNAKNSLFLQMNSLRAEDTAVY | |
| | YCARTRQLVGDYWGQGTLVTVSS | |
| 193 | EVQLVESGGGLVQPGRSLRLSCAASGFTFDNYAMHWVRQAPGKGL | REGN-VH-83 |
| | EWVSGIRWNSGSIGYADSVKGRFTISRDNAKNSLYLQMNSLRAEDT | |
| | ALYYCAKEGGYSGYRPGPFFDYWGQGTLVTVSS | |
| 194 | QVQLVQSGAEVKKPGASVKVSCKASGYTFTNYGISWVRQAPGQGL | REGN-VH-99 |
| | EWMGWISVYNGHTNYAQKLQGRVTMTTDTSTSTAYMELRSLRSDD | |
| | TAVYYCARGSGYDFDSWGQGTLVTVSS | |
| 195 | QVQLVQSGAEVKKPGASVKVSCKASRYTFTSYDINWVRQATGQGL | REGN-VH-115 |
| | EWMGWMNPNSGNTGYAQKFQGRVTMTRNTSTSTAYMELSSLRSE | |
| | DTAVYYCARVRRFFDYWGQGTLVTVSS | |
| 196 | QVQLVQSGPEVKKPGASVKVSCKASGYTFTNYGISWVRQAPGQGLE | REGN-VH-147 |
| | WMGWISVYNGNINYAQKLQGRVTMTTDTSTSTAYMDLRSLRSDDT | |
| | AVYYCARGSGYDFDYWGQGTLVTVSS | |
| 197 | QVQLVQSGAEVKKPGASVKVSCKDSAYTFNRYGISWVRQAPGQGL | REGN-VH-163 |
| | EWMGWISAYTGNTVYAQKLQGRVTMTTDNSTSTAYMELRSLRSDD | |
| | TAVYYCARDKSIFGVVRGFDYWGQGTLVTVSS | |
| 198 | AIQMTQSPSSLSASVGDRVTITCRASQGIRNALGWYQQKPGKAPKLL | REGN-VL-11 |
| | IYAASSLQSGVPSRFSGSGSGTDFTLTFSSLQPEDFATYYCLQDFNYP | |
| | YTFGQGTKLEIK | |
| 199 | DIQMTQSPSSVSASVGDRVTISCRASQGVSSWLAWYQQKPGNAPKL | REGN-VL-27 |
| | LISAASSIQSGVPSRFSGSGSGTDFTLTISSLQPEDFATYYCQQANSFPL | |
| | TFGGGTKVEIK | |
| 200 | DIQMTQSPSSVSASVGDRVTITCRASQGISSWLAWYQQKPGKAPKLL | REGN-VL-43 |
| | IYAASSFQSGVPSRFSGSGSGTDFTLTISSLQPEDFATYFCQQANSFPL | |
| | TFGGGTTVEIK | |
| 201 | DIQMTQSPSSVSASVGDRVTITCRASQDISIWLAWYQQSPGKAPKLLI | REGN-VL-59 |
| | NVASRLQSGVPSRFSGSGSGTDFTLTINSLQPEDFVTYYCQQANSFPI | |
| | TFGQGTRLATK | |
| 202 | DIQLTQSPSFLSASVGDRVTITCWASQGISSYLAWYQQKPGKAPKLLI | REGN-VE-75 |
| | FAASTLQSGVPSRFSGSGSGTEFTLTISSLQPEDFATYYCQQLNSYPLT | |
| | FGGGTKVEIR | |
| 203 | ElVMTQSPATESVSPGERATLSCRASQSVNYNLAWYQHKPGQAPRL | REGN-VE-91 |
| | LIYGASTRATGIPARFSGSGSGTEFTLTISSLQSEDFAVYYCQQYNNW | |
| | PLTFGGGTKVEIK | |
| 204 | AIQMTQSSSSLSASVGDRVTITCRASQAIRNALGWYQQKPGKAPKVL | REGN-VL-107 |
| | IYAASSLQSGIPSRFSGSGSGTDFTLTISSLQPEDFATYYCLQDYDYPY | |
| | TFGQGTKLEIK | |
| 205 | DIQLTQSPSFLSASVGDRVTITCWASQGIISYLAWYQQKPGKAPKLLI | REGN-VL-123 |
| | YAASTLHSGVPSRFSGSGSGTEFTLTISSLQPEDFATYYCHQLKSYPIT | |
| | FGQGTRLEIK | |
| 206 | AIQMTQSPSSLSASVGDRVTITCRASQDIRNALGWYQQKPGKAPKLL | REGN-VL-155 |
| | IYAASSLQSGVPSRFSGSASGTDFTLTISSLQPEDFAAYYCLQDYNYP | |
| | YTFGQGTKLEIK | |
| 207 | EIVMTQSPVIESLSPGERATLPCRASQSVSSSLAWYQQKAGQSPRLLI | REGN-VL-171 |
| | YGASTRATGIPARFSGSGSGTEFTLTISNLQSEDFAVYYCQQYNNWP | |
| | LTFGGGTKVEIK | |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

1. A method for treating atopic dermatitis (AD) or improving an AD-associated parameter in a subject, the method comprising:
(a) selecting a subject with moderate-to-severe or severe AD that is not adequately controlled by topical AD medications, wherein the subject is ≥6 months to <6 years of age; and
(b) administering to the subject one or more doses of an interleukin-4 receptor (IL-4R) antagonist, wherein the IL-4R antagonist is an anti-IL-4R antibody, or an antigen-binding fragment thereof, that comprises three HCDRs (HCDR1, HCDR2 and HCDR3) and three LCDRs (LCDR1, LCDR2 and LCDR3), wherein the HCDR1 comprises the amino acid sequence of SEQ ID NO:3, the HCDR2 comprises the amino acid sequence of SEQ ID NO:4, the HCDR3 comprises the amino acid sequence of SEQ ID NO:5, the LCDR1 comprises the amino acid sequence of SEQ ID NO:6, the LCDR2 comprises the amino acid sequence of SEQ ID NO:7, and the LCDR3 comprises the amino acid sequence of SEQ ID NO:8.

2. The method of claim 1, wherein the subject is a subject with severe AD.

3. The method of claim 1, wherein the subject is inadequately responsive to treatment with a topical corticosteroid (TCS) of medium or higher potency.

4. The method of claim 1, wherein the subject previously was administered a systemic AD medication.

5. The method of claim 1, wherein the subject is aged ≥6 months to <2 years.

6. The method of claim 1, wherein the subject is aged ≥2 to <6 years.

7. The method of claim 1, wherein the subject:
(i) has a baseline Investigator's Global Assessment (IGA) score=4;
(ii) has a baseline Eczema Area and Severity Index (EAST) score≥21; and/or
(iii) has a baseline Body Surface Area (BSA) affected by AD≥15%.

8. The method of claim 1, wherein the IL-4R antagonist is subcutaneously administered at a dose of 3 mg/kg.

9. The method of claim 1, wherein the IL-4R antagonist is subcutaneously administered at a dose of at least 6 mg/kg.

10. The method of claim 9, wherein the IL-4R antagonist is subcutaneously administered at a dose of 6 mg/kg.

11. The method of claim 1, wherein the IL-4R antagonist is subcutaneously administered in an amount that results in a total exposure to the IL-4R antagonist in the subject of at least 130 day·mg/L.

12. The method of claim 1, wherein the method comprises administering multiple doses of the IL-4R antagonist to the subject.

13. The method of claim 12, wherein the IL-4R antagonist is subcutaneously administered in an amount that results in a total exposure to the IL-4R antagonist in the subject of at least 130 day·mg/L for a period of at least two weeks.

14. The method of claim 1, wherein the IL-4R antagonist is administered to the subject once a week or once every two weeks.

15. The method of claim 1, wherein the subject has a concurrent atopic or allergic condition selected from the group consisting of allergic rhinitis, asthma, food allergy, allergic conjunctivitis, hives, chronic rhinosinusitis, nasal polyps, and eosinophilic esophagitis.

16. The method of claim 15, wherein the subject has a food allergy.

17. The method of claim 1, wherein the IL-4R antagonist is administered in combination with a topical AD medication.

18. The method of claim 17, wherein the topical AD medication is a medium-potency TCS or a low-potency TCS.

19. The method of claim 1, wherein treatment with the IL-4R antagonist results in a reduction in the level of one or more type 2 inflammatory biomarkers in the subject relative to a baseline value.

20. The method of claim 19, wherein treatment with the IL-4R antagonist results in a reduction in the level of serum TARC and/or serum total IgE in the subject relative to a baseline value.

21. The method of claim 1, wherein treatment with the IL-4R antagonist results in an improvement of one or more AD-associated parameters selected from:
(i) a reduction from baseline in IGA score to achieve an IGA score of 0 or 1 by week 4 after administration of the first dose of the IL-4R antagonist;
(ii) a reduction of at least 50% from baseline in an EASI score (EASI-50) by week 3 after administration of the first dose of the IL-4R antagonist;
(iii) a reduction of at least 75% from baseline in an EASI score (EASI-75) by week 3 after administration of the first dose of the IL-4R antagonist;
(iv) a reduction in percentage of BSA affected by AD to less than 40% of BSA by week 3 after administration of the first dose of the IL-4R antagonist; and
(v) a reduction of at 35% from baseline in BSA affected by AD by week 3 after administration of the first dose of the IL-4R antagonist.

22. The method of claim 1, wherein the AD-associated parameter is determined based on a caregiver reported assessment.

23. The method of claim 1, wherein treatment with the IL-4R antagonist results in an improvement in caregiver reported peak pruritus numerical rating scale (NRS) score.

24. The method of claim 1, wherein treatment with the IL-4R antagonist results in an improvement in itch.

25. The method of claim 24, wherein the improvement in itch is assessed by caregiver reported peak pruritus NRS score.

26. The method of claim 1, wherein the anti-IL-4R antibody or antigen-binding fragment thereof comprises a heavy chain variable region (HCVR) comprising the amino acid sequence of SEQ ID NO:1 and comprises a light chain variable region (LCVR) comprising the amino acid sequence of SEQ ID NO:2.

27. The method of claim 1, wherein the anti-IL-4R antibody comprises a heavy chain comprising the amino acid sequence of SEQ ID NO:9 and a light chain comprising the amino acid sequence of SEQ ID NO:10.

28. The method of claim 1, wherein the IL-4R antagonist is dupilumab or a bioequivalent thereof.

29. The method of claim 1, wherein the IL-4R antagonist is contained in a container selected from the group consisting of a glass vial, a syringe, a pre-filled syringe, a pen delivery device, and an autoinjector.

30. The method of claim 29, wherein the IL-4R antagonist is contained in a pre-filled syringe.

31. The method of claim 30, wherein the pre-filled syringe is a single-dose pre-filled syringe.

32. The method of claim 29, wherein the IL-4R antagonist is contained in an autoinjector.

33. The method of claim 29, wherein the IL-4R antagonist is contained in a pen delivery device.


SEQUENCE LISTING (207 sequences)

SEQ ID NO: 1 - protein, 124 aa, Artificial Sequence
  source: /note=“Description of Artificial Sequence Synthetic polypeptide”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVS

SEQ ID NO: 2 - protein, 112 aa, Artificial Sequence [sequence omitted]

SEQ ID NO: 3 - protein, 8 aa, Artificial Sequence
  source: /note=“Description of Artificial Sequence Synthetic peptide”
GFTFRDYA

SEQ ID NO: 4 - protein, 8 aa, Artificial Sequence
  source: /note=“Description of Artificial Sequence Synthetic peptide”
ISGSGGNT

SEQ ID NO: 5 - protein, 18 aa, Artificial Sequence
  source: /note=“Description of Artificial Sequence Synthetic peptide”
AKDRLSITIRPRYYGLDV

SEQ ID NO: 6 - protein, 11 aa, Artificial Sequence
  source: /note=“Description of Artificial Sequence Synthetic peptide”
QSLLYSIGYNY

SEQ ID NO: 7 - protein, 3 aa, Artificial Sequence
  source: /note=“Description of Artificial Sequence Synthetic peptide”
LGS

SEQ ID NO: 8 - protein, 9 aa, Artificial Sequence
  source: /note=“Description of Artificial Sequence Synthetic peptide”
MQALQTPYT

SEQ ID NO: 9 - protein, 452 aa, Artificial Sequence
  source: /note=“Description of Artificial Sequence Synthetic polypeptide”
EVQLVESGGGLEQPGGSLRLSCAGSGFTFRDYAMTWVRQAPGKGLEWVSSISGSGGNTYYADSVKGRFTISRDNSKNTLYLQMNSLRAEDTAVYYCAKDRLSITIRPRYYGLDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 219 aa, Artificial Sequence
  source: /note=“Description of Artificial Sequence Synthetic polypeptide”
DIVMTQSPLSLPVTPGEPASISCRSSQSLLYSIGYNYLDWYLQKSGQSPQLLIYLGSNRASGVPDRFSGSGSGTDFTLKISRVEAEDVGFYYCMQALQTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 207 aa, Homo sapiens
MKVLQEPTCVSDYMSISTCEWKMNGPTNCSTELRLLYQLVFLLSEAHTCIPENNGGAGCVCHLLMDDVVSADNYTLDLWAGQQLLWKGSFKPSEHVKPRAPGNLTVHTNVSDTLLLTWSNPYPPDNYLYNHLTYAVNIWSENDPADFRIYNVTYLEPSLRIAASTLKSGISYRARVRAWAQCYNTTWSEWSPSTKWHNSYREPFEQH

SEQ ID NO: 12 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRATGIPDRFSGSGSGTDFTLTISRLEPEDFAVYYCQQYGSSPPWTFGQGTKVEIK

SEQ ID NO: 13 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPPWTFGQGTKVEIK

SEQ ID NO: 14 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRAPGIPDRFSGSGSGTDFTLTISRLEPEDFAVYYCQQYGSSPPWTFGQGTKVEIK

SEQ ID NO: 15 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIYGASSRATGIPDRFSGSGSGTDFTLTISRLEPEDFAVYYCQQYGSSPPWTFGQGTKVEIK

SEQ ID NO: 16 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRAPGIPDRFSGSGSGTDFTLTISRLEPEDFAVYYCQQYGSSPPWTFGQGTKVEIK

SEQ ID NO: 17 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRAPGIPDRFSGSGSGTDFTLTISRLEPEDFAVYYCQQYGSSPPWTFGQGTKVEIK

SEQ ID NO: 18 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSPPWTFGQGTKVEIK

SEQ ID NO: 19 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSAGWTFGQGTKVEIK

SEQ ID NO: 20 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRATGIPDRFSGSGSGTDFTLTISRLEPEDFAVYYCQQYDHSAGWTFGQGTKVEIK

SEQ ID NO: 21 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSPPWTFGQGTKVEIK

SEQ ID NO: 22 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSPPWTFGQGTKVEIK

SEQ ID NO: 23 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSPPWTFGQGTKVEIK

SEQ ID NO: 24 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRAPGIPDRFSGSGSGTDFTLTISRLEPEDFAVYYCQQYDHSAGWTFGQGTKVEIK

SEQ ID NO: 25 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRAPGIPDRFSGSGSGTDFTLTISRLEPEDFAVYYCQQYDHSAGWTFGQGTKVEIK

SEQ ID NO: 26 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIYGASSRATGIPDRFSGSGSGTDFTLTISRLEPEDFAVYYCQQYDHSAGWTFGQGTKVEIK

SEQ ID NO: 27 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRAPGIPDRFSGSGSGTDFTLTISRLEPEDFAVYYCQQYDHSAGWTFGQGTKVEIK

SEQ ID NO: 28 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQQYDHSAGWTFGQGTKVEIK

SEQ ID NO: 29 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIFGASSRATGIPDRFSGSGSGTDFTLTISRLEPEDFAVYYCQQYDHSAGWTFGQGTKVEIK

SEQ ID NO: 30 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIFGASSRATGIPDRFSGSGSGTDFTLTISRLEPEDFAVYYCQQYGSSPPWTFGQGTKVEIK

SEQ ID NO: 31 - protein, 109 aa, Artificial Sequence
  source: /note=“Description of Artificial Sequence Synthetic polypeptide”
EIVLTQSPGTLSLSPGERATLSCRASQSVSNSYLAWYQQKPGQAPRLLIYGASSRAPGIPDRFSGSGSGTDFTLTISRLEPEDFAVYYCQQYDHSAGWTFGQGTKVEIK

SEQ ID NO: 32 - protein, 115 aa, Artificial Sequence
  source: /note=“Description of Artificial Sequence Synthetic polypeptide”
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 33 - protein, 115 aa, Artificial Sequence
  source: /note=“Description of Artificial Sequence Synthetic polypeptide”
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 34 - protein, 115 aa, Artificial Sequence
  source: /note=“Description of Artificial Sequence Synthetic polypeptide”
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 35 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFDYWGQGTLVTVSS

SEQ ID NO: 36 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFDYWGQGTLVTVSS

SEQ ID NO: 37 - protein, 115 aa, Artificial Sequence
  source: /note=“Description of Artificial Sequence Synthetic polypeptide”
EVQLVESGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 38 - protein, 115 aa, Artificial Sequence
  source: /note=“Description of Artificial Sequence Synthetic polypeptide”
EVQLVESGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 39 - protein, 115 aa, Artificial Sequence
  source: /note=“Description of Artificial Sequence Synthetic polypeptide”
EVQLVQSGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 40 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 41 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 42 - protein, 115 aa, Artificial Sequence
  source: /note=“Description of Artificial Sequence Synthetic polypeptide”
EVQLVESGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 43 - protein, 115 aa, Artificial Sequence
  source: /note=“Description of Artificial Sequence Synthetic polypeptide”
EVQLVQSGGGLVQPGGSLRLSCAGSGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 44 - protein, 115 aa, Artificial Sequence
  source: /note=“Description of Artificial Sequence Synthetic polypeptide”
EVQLVQSGGGLVHPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 45 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 46 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 47 - protein, 115 aa, Artificial Sequence
  source: /note=“Description of Artificial Sequence Synthetic polypeptide”
EVQLVQSGGGLVHPGRSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 48 - protein, 115 aa, Artificial Sequence
  source: /note=“Description of Artificial Sequence Synthetic polypeptide”
EVQLVQSGGGLVHPGGSLRLTCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 49 - protein, 115 aa, Artificial Sequence
  source: /note=“Description of Artificial Sequence Synthetic polypeptide”
EVQLVQSGGGLVHPGGSLRLSCAGSGFTFSRNAMHWVRQAPGKGLEWVSGIGTGGATNYADSVKGRFTISRDNAKNSLYLQMNSLRAEDMAVYYCARGRYYFDYWGQGTLVTVSS

SEQ ID NO: 50 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGEGLEWVSGIGTGGATNYADSVKGRFTISRDNAKNSLYLQMNSLRAEDMAVYYCARGRYYFDYWGQGTLVTVSS

SEQ ID NO: 51 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDEAKNSLYLQMNSLRAEDMAVYYCARGRYYFDYWGQGTLVTVSS

SEQ ID NO: 52 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAGDMAVYYCARGRYYFDYWGQGTLVTVSS

SEQ ID NO: 53 - protein, 115 aa, Artificial Sequence
  source: /note=“Description of Artificial Sequence Synthetic polypeptide”
EVQLVQSGGGLVHPGGSLRLSCAGSGFTFDDYAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 54 - protein, 115 aa, Artificial Sequence
  source: /note=“Description of Artificial Sequence Synthetic polypeptide”
EVQLVQSGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 55 - protein, 115 aa, Artificial Sequence
  source: /note=“Description of Artificial Sequence Synthetic polypeptide”
EVQLVESGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 56 - protein, 115 aa, Artificial Sequence
  source: /note=“Description of Artificial Sequence Synthetic polypeptide”
EVQLVESGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 57 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATNYADSVKGRFTISRDNAKNSLYLQMNSLRAEDTAVYYCARGRYYFPWWGQGTLVTVSS

SEQ ID NO: 58 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATSYADSVKGRFTISRDNAKNSLYLQMNSLRAEDMAVYYCARGRYYFPWWGQGTLVTVSS

SEQ ID NO: 59 - protein, 115 aa, Artificial Sequence
  source: /note=“Description of Artificial Sequence Synthetic polypeptide”
EVQLVQSGGGLVHPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 60 - protein, 115 aa, Artificial Sequence
  source: /note=“Description of Artificial Sequence Synthetic polypeptide”
EVQLVQSGGGLVQPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 61 - protein, 115 aa, Artificial Sequence
  source: /note=“Description of Artificial Sequence Synthetic polypeptide”
EVQLVESGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 62 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 63 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATNYADSVKGRFTISRDNAKNSLYLQMNSLRAEDMAVYYCARGRYYFPWWGQGTLVTVSS

SEQ ID NO: 64 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFDYWGQGTLVTVSS

SEQ ID NO: 65 - protein, 115 aa, Artificial Sequence
  source: /note=“Description of Artificial Sequence Synthetic polypeptide”
EVQLVQSGGGLVHPGGSLRLSCAGSGFTFSRNAMFWVRQAPGKGLEWVSGIGTGGATNYADSVKGRFTISRDNAKNSLYLQMNSLRAEDMAVYYCARGRYYFDYWGQGTLVTVSS

SEQ ID NO: 66 - protein, 115 aa, Artificial Sequence
  source: /note=“Description of Artificial Sequence Synthetic polypeptide”
EVQLVESGGGLVQPGGSLRLSCAASGFTFSRNAMFWVRQAPGKGLEWVSGIGTGGATSYADSVKGRFTISRDNAKNSLYLQMNSLRAEDTAVYYCARGRYYFPWWGQGTLVTVSS

SEQ ID NO: 67 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLDYWGKGTLVTVSS

SEQ ID NO: 68 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSLSANYVFGTGTKLTVL

SEQ ID NO: 69 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYNWGKGTLVTVSS

SEQ ID NO: 70 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSQPPNPLFGTGTKLTVL

SEQ ID NO: 71 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKLLKNPWGKGTLVTVSS

SEQ ID NO: 72 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWFGTPASNYVFGTGTKLTVL

SEQ ID NO: 73 - protein, 116 aa, Artificial Sequence; identical to SEQ ID NO: 69
  source: /note=“Description of Artificial Sequence Synthetic polypeptide”

SEQ ID NO: 74 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSSPPQPIFGTGTKLTVL

SEQ ID NO: 75 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWLYDWGKGTLVTVSS

SEQ ID NO: 76 - protein, 111 aa, Artificial Sequence; identical to SEQ ID NO: 74
  source: /note=“Description of Artificial Sequence Synthetic polypeptide”

SEQ ID NO: 77 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 78 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTYHPIFGTGTKLTVL

SEQ ID NO: 79 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQYWGKGTLVTVSS

SEQ ID NO: 80 - protein, 111 aa, Artificial Sequence; identical to SEQ ID NO: 74
  source: /note=“Description of Artificial Sequence Synthetic polypeptide”

SEQ ID NO: 81 - protein, 116 aa, Artificial Sequence; identical to SEQ ID NO: 79
  source: /note=“Description of Artificial Sequence Synthetic polypeptide”

SEQ ID NO: 82 - protein, 111 aa, Artificial Sequence; identical to SEQ ID NO: 78
  source: /note=“Description of Artificial Sequence Synthetic polypeptide”

SEQ ID NO: 83 - protein, 116 aa, Artificial Sequence; identical to SEQ ID NO: 69
  source: /note=“Description of Artificial Sequence Synthetic polypeptide”

SEQ ID NO: 84 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTMYPLFGTGTKLTVL

SEQ ID NO: 85 - protein, 116 aa, Artificial Sequence; identical to SEQ ID NO: 75
  source: /note=“Description of Artificial Sequence Synthetic polypeptide”

SEQ ID NO: 86 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVLTPIFGTGTKLTVL

SEQ ID NO: 87 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWFYDWGKGTLVTVSS

SEQ ID NO: 88 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPSMIPLFGTGTKLTVL

SEQ ID NO: 89 - protein, 116 aa, Artificial Sequence; identical to SEQ ID NO: 87
  source: /note=“Description of Artificial Sequence Synthetic polypeptide”

SEQ ID NO: 90 - protein, 111 aa, Artificial Sequence; identical to SEQ ID NO: 84
  source: /note=“Description of Artificial Sequence Synthetic polypeptide”

SEQ ID NO: 91 - protein, 116 aa, Artificial Sequence; identical to SEQ ID NO: 75
  source: /note=“Description of Artificial Sequence Synthetic polypeptide”

SEQ ID NO: 92 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTLQPLFGTGTKLTVL

SEQ ID NO: 93 - protein, 116 aa, Artificial Sequence; identical to SEQ ID NO: 69
  source: /note=“Description of Artificial Sequence Synthetic polypeptide”

SEQ ID NO: 94 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPPTKPLFGTGTKLTVL

SEQ ID NO: 95 - protein, 116 aa, Artificial Sequence; identical to SEQ ID NO: 69
  source: /note=“Description of Artificial Sequence Synthetic polypeptide”

SEQ ID NO: 96 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTHRHPLFGTGTKLTVL

SEQ ID NO: 97 - protein, 116 aa, Artificial Sequence; identical to SEQ ID NO: 69
  source: /note=“Description of Artificial Sequence Synthetic polypeptide”

SEQ ID NO: 98 - protein, 111 aa, Artificial Sequence; identical to SEQ ID NO: 78
  source: /note=“Description of Artificial Sequence Synthetic polypeptide”

SEQ ID NO: 99 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKWWWQHWGKGTLVTVSS

SEQ ID NO: 100 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVDRPIFGTGTKLTVL

SEQ ID NO: 101 - protein, 116 aa, Artificial Sequence; identical to SEQ ID NO: 99
  source: /note=“Description of Artificial Sequence Synthetic polypeptide”

SEQ ID NO: 102 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTTPMPVFGTGTKLTVL

SEQ ID NO: 103 - protein, 116 aa, Artificial Sequence; identical to SEQ ID NO: 99
  source: /note=“Description of Artificial Sequence Synthetic polypeptide”

SEQ ID NO: 104 - protein, 111 aa, Artificial Sequence; identical to SEQ ID NO: 78
  source: /note=“Description of Artificial Sequence Synthetic polypeptide”

SEQ ID NO: 105 - protein, 116 aa, Artificial Sequence; identical to SEQ ID NO: 77
  source: /note=“Description of Artificial Sequence Synthetic polypeptide”

SEQ ID NO: 106 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTVWEWPFGTGTKLTVL

SEQ ID NO: 107 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSASYAQKFQGRVTMTRDTSTSTVYMELSSLRSEDTAVYYCARGKYWMYDWGKGTLVTVSS

SEQ ID NO: 108 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEAVYFCGTWDTSTVWEWPFGTGTKLTVL

SEQ ID NO: 109 - protein, 116 aa, Artificial Sequence; identical to SEQ ID NO: 77
  source: /note=“Description of Artificial Sequence Synthetic polypeptide”

SEQ ID NO: 110 - protein, 111 aa, Artificial Sequence
  source: /note=“Description of Artificial Sequence Synthetic polypeptide”
QPVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 111 - protein, 116 aa, Artificial Sequence
  source: /note=“Description of Artificial Sequence Synthetic polypeptide”
QVQLVQSGAEVRKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 112 - protein, 111 aa, Artificial Sequence
  source: /note=“Description of Artificial Sequence Synthetic polypeptide”
QSVLTQPPSVSAAPGQKVTISCSGGSSNIGNNYVSWYQQLPGTAPKLLIYDNNKRPPGIPDRFSGSKSGTSATLAITGLQTGDEADYYCGTWDTSTVWEWPFGTGTKLTVL

SEQ ID NO: 113 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPRGGSTSYAQKFQGRVTMTRDTSTSTVYMELSSLRSEDTAVYYCARGKYWMYDWGKGTLVTVSS

SEQ ID NO: 114 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 115 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPRGGSASYAQKFQGRVSMTRDTSTSTVYMELSSLRSEDTAVYYCARGKYWMYDWGKGTLVTVSS

SEQ ID NO: 116 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTTATLAITGLQTGDEADYYCGTWVTSTVWEWPFGTGTKLTVL

SEQ ID NO: 117 - protein, 116 aa, Artificial Sequence; identical to SEQ ID NO: 77
  source: /note=“Description of Artificial Sequence Synthetic polypeptide”

SEQ ID NO: 118 - protein, 111 aa, Artificial Sequence; identical to SEQ ID NO: 114
  source: /note=“Description of Artificial Sequence Synthetic polypeptide”

SEQ ID NO: 119 - protein, 116 aa, Artificial Sequence
  source: /note=“Description of Artificial Sequence Synthetic polypeptide”
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTQVTVSS

SEQ ID NO: 120 - protein, 111 aa, Artificial Sequence
  source: /note=“Description of Artificial Sequence Synthetic polypeptide”
QSVLTQPPLVSAAPGQKVTISCSGGSSNIGNSYVSWYQRLPGTAPKLLIYDNNKRPSGIPDRFSGSKSGTSATLAITGLQTGDEADYYCGTWDTSTVWEWPFGTGTKLTVL

SEQ ID NO: 121 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGNGTLVTVSS

SEQ ID NO: 122 - protein, 111 aa, Artificial Sequence
  source: /note=“Description of Artificial Sequence Synthetic polypeptide”
LPVLTQPPSVSAAPGQKVTISCSGGSSSIGNSYVSWYQQLPGAAPKLLIYDNNKRPSGIPDRFSGFRSGTSATLAITGLQTGDEADYYCGTWDTSPVWEWPFGTGTKLTVL

SEQ ID NO: 123 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTRVTVSS

SEQ ID NO: 124 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSPVWEWPFGTGTKLTVL

SEQ ID NO: 125 - protein, 116 aa, Artificial Sequence; identical to SEQ ID NO: 77
  source: /note=“Description of Artificial Sequence Synthetic polypeptide”

SEQ ID NO: 126 - protein, 111 aa, Artificial Sequence
  source: /note=“Description of Artificial Sequence Synthetic polypeptide”
QSVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTVWEWPFGTGTKLTVL

SEQ ID NO: 127 - protein, 116 aa, Artificial Sequence; identical to SEQ ID NO: 77
  source: /note=“Description of Artificial Sequence Synthetic polypeptide”

SEQ ID NO: 128 - protein, 111 aa, Artificial Sequence
  source: /note=“Description of Artificial Sequence Synthetic polypeptide”
QSVLTQPPSVSAAPGQKVTISCSGGSSSIGNSYVSWYQQLPGTAPKLLIYDNNKRPSGIPDRFSGSKSGTSATLAITGLQTGDEADYYCGTWATSPVWEWPFGTGTKLTVL

SEQ ID NO: 129 - protein, 116 aa, Artificial Sequence; identical to SEQ ID NO: 77
  source: /note=“Description of Artificial Sequence Synthetic polypeptide”

SEQ ID NO: 130 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYFCGTWDTSTAWEWPFGTGTKLTVL

SEQ ID NO: 131 - protein, 116 aa, Artificial Sequence
  source: /note=“Description of Artificial Sequence Synthetic polypeptide”
QVQLVQSGAEEKKPGASVKVSCKASGYAFTSYYMHWARQAPGQGLEWMGIINPSGGSTSYAQKFQGRVTMTRDTSTSTVYMELSSLRSEDTAVYYCARGKYWMYDWGKGTLVTVSS

SEQ ID NO: 132 - protein, 111 aa, Artificial Sequence
  source: /note=“Description of Artificial Sequence Synthetic polypeptide”
QSALTQPPSVSAAPGQKVTISCSGGSSNIGNSYVSWYQQLPGTAPKLLIYDNNKRPSGIPDRFSGSKSGTSATLAITGLQTGDEADYFCGTWDTSTVWEWPFGTGTKLTVL

SEQ ID NO: 133 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPSGGSTSYAQKFQGRVSMTRDTSTSTVYMELSSLRSEDTAVYYCARGKYWMYDWGKGTLVTVSS

SEQ ID NO: 134 - protein, 111 aa, Artificial Sequence; identical to SEQ ID NO: 114
  source: /note=“Description of Artificial Sequence Synthetic polypeptide”

SEQ ID NO: 135 - protein, 116 aa, Artificial Sequence; identical to SEQ ID NO: 77
  source: /note=“Description of Artificial Sequence Synthetic polypeptide”

SEQ ID NO: 136 - protein, 111 aa, Artificial Sequence; identical to SEQ ID NO: 124
  source: /note=“Description of Artificial Sequence Synthetic polypeptide”

SEQ ID NO: 137 - protein, 116 aa, Artificial Sequence; identical to SEQ ID NO: 107
  source: /note=“Description of Artificial Sequence Synthetic polypeptide”

SEQ ID NO: 138 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDSSTVWEWPFGTGTKLTVL

SEQ ID NO: 139 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWMGIINPRGGSTSYAQKFQGRVAMTRDTSTSTVYMELSSLRPEDTAVYYCARGKYWMYDWGKGTLVTVSS

SEQ ID NO: 140 - protein, 111 aa, Artificial Sequence
  source: /note=“Description of Artificial Sequence Synthetic polypeptide”
QSVLTQPPSVSAAPGQKVTISCSGGGSSIGNSYVSWYQQLPGTAPKLLIYDNNKRPSGVPDRFSGSKSGTSATLAITGLQTGDEADYYCGTWDTSPVWEWPFGTGTKLTVL

SEQ ID NO: 141 - protein, 116 aa, Artificial Sequence; identical to SEQ ID NO: 107
  source: /note=“Description of Artificial Sequence Synthetic polypeptide”

SEQ ID NO: 142 - protein, 111 aa, Artificial Sequence; identical to SEQ ID NO: 114
  source: /note=“Description of Artificial Sequence Synthetic polypeptide”

SEQ ID NO: 143 - protein, 116 aa, Artificial Sequence; identical to SEQ ID NO: 113
  source: /note=“Description of Artificial Sequence Synthetic polypeptide”

SEQ ID NO: 144 - protein, 111 aa, Artificial Sequence
  source: /note=“Description of Artificial Sequence Synthetic polypeptide”
QSVLTQPPSVSAAPGQKVTISCSGGSSNIGNSYVSWYQQLPGTAPKLLIYDNNKRPSGIPDRFSGSKSGTSATLAITGLQTGDEADYYCGTWDTSTAWEWPFGTGTKLTVL

SEQ ID NO: 145 - protein, 116 aa, Artificial Sequence; identical to SEQ ID NO: 77
  source: /note=“Description of Artificial Sequence Synthetic polypeptide”

SEQ ID NO: 146 - protein, 111 aa, Artificial Sequence; identical to SEQ ID NO: 138
  source: /note=“Description of Artificial Sequence Synthetic polypeptide”

SEQ ID NO: 147 - protein, 116 aa, Artificial Sequence
  source: /note=“Description of Artificial Sequence Synthetic polypeptide”
QVQLVQSGAEVRKPGASVKVSCKASGYAFTSYYMHWARQAPGQGLEWMGIINPSGGSTSYAQKFQGRVTMTRDTSTSTVYMELSSLRPEDTAVYYCARGKYWMYDWGKGTLVTVSG

SEQ ID NO: 148 - protein, 111 aa, Artificial Sequence
  source: /note=“Description of Artificial Sequence Synthetic polypeptide”
QSVLTQPPSVSAAPGQKVTISCSGGSTNIGNSYVSWYQRLPGTAPKLLIYDNNKRPPGIPDRFSGSKSGTSATLAITGLQTGDEADYYCGTWDTSTVWEWPFGTGTKLTVL

SEQ ID NO: 149 - protein, 116 aa, Artificial Sequence
  source: /note=“Description of Artificial Sequence Synthetic polypeptide”
QVQLVQSGAEVKKPGASVKVSCKASGYAFTSYYMHWARQAPGQGLEWVGIINPSGGSTSYAQKFQGRVTMTRDTSTSTVYMELSSLRSGDTAVYYCARGKYWMYDWGKGTLVTVSS

SEQ ID NO: 150 - protein, 111 aa, Artificial Sequence
  source: /note=“Description of Artificial Sequence Synthetic polypeptide”
QAVLTQPPSVSAAPGQKVTISCSGGSSNIGNSYVSWYQRLPGAAPKLLIYDNNKRPSGIPDRFSGSKSGTSATLAITGLQTGDEADYYCGTWDTSTGWEWPFGTGTKLTVL

SEQ ID NO: 151 - protein, 116 aa, Artificial Sequence
  source: /note=“Description of Artificial Sequence Synthetic polypeptide”
QVQLVQSGAEVKKPGASVKVSCKASGYAFTSYYMHWVRQAPGQGLEWMGIINPRGGSTSYAQKFQGRVTMTRDTSTSTVYMELSSLRSEDTAVYYCARGKYWMYDWGKGTLVTVSS

SEQ ID NO: 152 - protein, 111 aa, Artificial Sequence
  source: /note=“Description of Artificial Sequence Synthetic polypeptide”
QSVLTQPPSVSAAPGQKVTISCSGGGSSIGNSYVSWYQQLPGTAPKLLIYDNNKRPSGIPDRFSGSKSGTSATLGITGLQTGDEADYYCGTWDTSPVWEWPFGTGTKLTVL

SEQ ID NO: 153 - protein, 116 aa, Artificial Sequence
  source: /note=“Description of Artificial Sequence Synthetic polypeptide”
EVQLLESGGGLVQPGGSLRLSCAVSGFTFSNYAMSWVRQAPGKGLEWVSAISSGGGNIYYADSVKGRFTISRDNSKNTLYLQMNSLRAEDTAVYYCAKLRRYFDYWGQGTLVTVSS

SEQ ID NO: 154 - protein, 121 aa, Artificial Sequence
  source: /note=“Description of Artificial Sequence Synthetic polypeptide”
EVQLLESGGGLVQPGGSLRLSCAASGFTFSDYAMSWVRQAPGKGLEWVSAISSGGSSIYYADSVKGRFTISRDNSKNTLHLQMNSLRAEDTAVYYCARGPQRSATAVFDYWGQGTLVTVSS

SEQ ID NO: 155 - protein, 127 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSWISPNSGNIYYADSVKGRFTISRDNSKNTLYLQMNSLRAEDTAVYYCARRPLSAAWSHSSYYNAMDVWGQGTLVTVSS

SEQ ID NO: 156 - protein, 116 aa, Artificial Sequence
  source: /note=“Description of Artificial Sequence Synthetic polypeptide”
EVQLLESGGGLVQPGGSLRLSCAASGFTFSGYAMSWVRQAPGKGLEWVSLISHSGSNTYYADSVKGRFTISRDNSKNTLYLQMNSLRAEDTAVYYCARPHRAFDYWGQGTLVTVSS

SEQ ID NO: 157 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSGISHGSGSIYYADSVKGRFTISRDNSKNTLYLQMNSLRAEDTAVYYCARPHRAFDYWGQGTLVTVSS

SEQ ID NO: 158 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSGISHGNGSIYYADSVKGRFTISRDNSKNTLYLQMNSLRAEDTAVYYCAKTGRHFDYWGQGTLVTVSS

SEQ ID NO: 159 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSSISPSGSSIYYADSVKGRFTISRDNSKNTLYLQMNSLRAEDTAVYYCARSYRAFDYWGQGTLVTVSS

SEQ ID NO: 160 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAISPSGGSIYYADSVKGRFTISRDNSKNTLYLQMNSLRAEDTAVYYCARAKRAFDYWGQGTLVTVSS

SEQ ID NO: 161 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAISPGSGSTYYADSVKGRFTISRDNSKNTLYLQMNSLRAEDTAVYYCAKFRRHFDYWGQGTLVTVSS

SEQ ID NO: 162 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAISSGGGNIYYADSVKGRFTISRDNSKNTLYLQMNSLRAEDTAVYYCARVHRAFDYWGQGTLVTVSS

SEQ ID NO: 163 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAITSSGRSIYYADSVKGRFTISRDNSKNTLYLQMNSLRAEDTAVYYCARVHRAFDYWGQGTLVTVSS

SEQ ID NO: 164 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAITSSGANIYYADSVKGRFTISRDNSKNTLYLQMNSLRAEDTAVYYCARVHRAFDYWGQGTLVTVSS

SEQ ID NO: 165 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAITSSGGNIYYADSVKGRFTISRDNSKNTLYLQMNSLRAEDTAVYYCARVHRAFDYWGQGTLVTVSS

SEQ ID NO: 166 - protein, 116 aa, Artificial Sequence
  source: /note=“Description of Artificial Sequence Synthetic polypeptide”
EVQLLESGGGLVQPGGSLRLSCAASGFTFSNYAMSWVRQAPGKGLEWVSAITAGGGSIYYADSVKGRFTISRDNSKNTLYLQMNSLRAEDTAVYYCARVHRAFDYWGQGTLVTVSS

SEQ ID NO: 167 - protein, 116 aa, Artificial Sequence
  source: /note=“Description of Artificial Sequence Synthetic polypeptide”
EVQLLESGGGLVQPGGSLRLSCAASGFTFSRHAMAWVRQAPGKGLEWVSAITSSGRSIYYADSVKGRFTISRDNSKNTLYLQMNSLRAEDTAVYYCARVHRAFDYWGQGTLVTVSS

SEQ ID NO: 168 - protein, 110 aa, Artificial Sequence
  source: /note=“Description of Artificial Sequence Synthetic polypeptide”
QSVLTQPPSASGTPGQRVTISCSGSSSNIGNNYVNWYQQLPGTAPKLLIYDNSHRPSGVPDRFSGSKSGTSASLAISGLRSEDEADYYCGTWDASLSAYVFGGGTKLTVL

SEQ ID NO: 169 - protein, 110 aa, Artificial Sequence
  source: /note=“Description of Artificial Sequence Synthetic polypeptide”
QSVLTQPPSASGTPGQRVTISCSGSSSNIGNNNVSWYQQLPGTAPKLLIYANSKRPSGVPDRFSGSKSGTSASLAISGLRSEDEADYYCGSWDDSLSAYVFGGGTKLTVL

SEQ ID NO: 170 - protein, 110 aa, Artificial Sequence
  source: /note=“Description of Artificial Sequence Synthetic polypeptide”
QSVLTQPPSAPGTPGQRVTISCTGSSSNIGSNSVNWYQQLPGTAPKLLIYDDSHRPSGVPDRFSGSKSGTSASLAISGLRSEDEADYYCDAWDSSLSAYVFGGGTKLTVL

SEQ ID NO: 171 - protein, 110 aa, Artificial Sequence
  source: /note=“Description of Artificial Sequence Synthetic polypeptide”
QSVLTQPPSASGTPGQRVTLSCTGSSSNIGSNYVSWYQQLPGTAPKLLIYADSQRPSGVPDRFSGSKSGTSASLAISGLRSEDEADYYCGTWDDSLSGYVFGGGTKLTVL

SEQ ID NO: 172 - protein, 110 aa, Artificial Sequence
  source: /note=“Description of Artificial Sequence Synthetic polypeptide”
QSVLTQPPSASGTPGQRVTISCSSSSSNIGSNYVSWYQQLPGTAPKLLIYSDSHRPSGVPDRFSGSKSGTSASLAISGLRSEDEADYYCGSWDYSLSAYVFGGGTKLTVL

SEQ ID NO: 173 - protein, 110 aa, Artificial Sequence
  source: /note=“Description of Artificial Sequence Synthetic polypeptide”
QSVLTQPPSASGTPGQRVTISCTGSSSNIGNNTVSWYQQLPGTAPKLLIYDNSHRPSGVPDRFSGSKSGTSASLAISGLQSEDEADYYCGSWDYSLSAYVFGGGTKLTVL

SEQ ID NO: 174 - protein, 110 aa, Artificial Sequence
  source: /note=“Description of Artificial Sequence Synthetic polypeptide”
QSVLTQPPSASGTPGQRVTISCTGSSSNIGNNDVNWYQQLPGTAPKLLIYYDSQRPSGVPDRFSGSKSGTSASLAISGLRSEDEADYYCATWDASLSAYVFGGGTKLTVL

SEQ ID NO: 175 - protein, 110 aa, Artificial Sequence
  source: /note=“Description of Artificial Sequence Synthetic polypeptide”
QSVLTQPPSASGTPGQRVTISCSGSSSNIGSNAVNWYQQLPGTAPKLLIYYDNQRPSGVPDRFSGSKSGTSASLAISGLRSEDEADYYCGTWDDSLNGYVFGGGTKLTVL

SEQ ID NO: 176 - protein, 110 aa, Artificial Sequence
  source: /note=“Description of Artificial Sequence Synthetic polypeptide”
QSVLTQPPSASGTPGQRVTISCSGSSSNIGNNAVTWYQQLPGTAPKLLIYDDSHRPSGVPDRFSGSKSGTSASLAISGLRSEDEADYYCGSWDYSLSAYVFGGGTKLTVL

SEQ ID NO: 177 - protein, 110 aa, Artificial Sequence
  source: /note=“Description of Artificial Sequence Synthetic polypeptide”
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLSGYVLGGGTKLTVL

SEQ ID NO: 178 - protein, 110 aa, Artificial Sequence; identical to SEQ ID NO: 177
  source: /note=“Description of Artificial Sequence Synthetic polypeptide”

SEQ ID NO: 179 - protein, 110 aa, Artificial Sequence
  source: /note=“Description of Artificial Sequence Synthetic polypeptide”
QSVLTQPPSASGTPGQRVTISCSGSSSNIGSNTFNWYQQLPGTAPKLLIYADSHRPSGVPDRFSGSKSGTSASLAISGLRSEDEADYYCGTWDYSLRGYVLGGGTKLTVL

SEQ ID NO: 180 - protein, 110 aa, Artificial Sequence
  source: /note=“Description of Artificial Sequence Synthetic polypeptide”
QSVLTQPPSASGTPGQRVTISCSGSSSNIGSNTFNWYQQLPGTAPKLLIYADSHRPSGVPDRFSGSKSGTSASLAISGLRSEDEADYYCGYWDYSLSGYVLGGGTKLTVL

SEQ ID NO: 181 - protein, 110 aa, Artificial Sequence; identical to SEQ ID NO: 177
  source: /note=“Description of Artificial Sequence Synthetic polypeptide”

SEQ ID NO: 182 - protein, 111 aa, Artificial Sequence
  source: /note=“Description of Artificial Sequence Synthetic polypeptide”
QSVLTQPPSASGTPGQRVTISCSGSSANSRTDGFNWYQQLPGTAPKLLIYADSHRPSGVPDRFSGSKSGTSASLAISGLRSEDEADYYCGTWDYSLSGYVLGGGTKLTVLG

SEQ ID NO: 183 - protein, 111 aa, Artificial Sequence
  source: /note=“Description of Artificial Sequence Synthetic polypeptide”
QSVLTQPPSASGTPGQRVTISCSGSAQFGSRDNFNWYQQLPGTAPKLLIYADSHRPSGVPDRFSGSKSGTSASLAISGLRSEDEADYYCGTWDYSLSGYVLGGGTKLTVLG

SEQ ID NO: 184 - protein, 111 aa, Artificial Sequence
  source: /note=“Description of Artificial Sequence Synthetic polypeptide”
QSVLTQPPSASGTPGQRVTISCSGSTKQMHNYQFNWYQQLPGTAPKLLIYADSHRPSGVPDRFSGSKSGTSASLAISGLRSEDEADYYCGTWDYSLSGYVLGGGTKLTVLG

SEQ ID NO: 185 - protein, 111 aa, Artificial Sequence
  source: /note=“Description of Artificial Sequence Synthetic polypeptide”
QSVLTQPPSASGTPGQRVTISCSGSLLRGENLQFNWYQQLPGTAPKLLIYADSHRPSGVPDRFSGSKSGTSASLAISGLRSEDEADYYCGTWDYSLSGYVLGGGTKLTVLG

SEQ ID NO: 186 - protein, 111 aa, Artificial Sequence
  source: /note=“Description of Artificial Sequence Synthetic polypeptide”
QSVLTQPPSASGTPGQRVTISCSGSPLFPDSGSFNWYQQLPGTAPKLLIYADSHRPSGVPDRFSGSKSGTSASLAISGLRSEDEADYYCGTWDYSLSGYVLGGGTKLTVLG

SEQ ID NO: 187 - protein, 111 aa, Artificial Sequence
  source: /note=“Description of Artificial Sequence Synthetic polypeptide”
QSVLTQPPSASGTPGQRVTISCSGSAALDLSPSFNWYQQLPGTAPKLLIYADSHRPSGVPDRFSGSKSGTSASLAISGLRSEDEADYYCGTWDYSLSGYVLGGGTKLTVLG

SEQ ID NO: 188 - protein, 117 aa, Artificial Sequence
  source: /note=“Description of Artificial Sequence Synthetic polypeptide”
QVQLVQSGAEVKKPGASVKVSCKASGYTFTNYGISWVRQAPGQGLEWMGWISVYNGKTNYAQKLQGRVTMTTDTSTTTAYMEMRSLRSDDTAVYYCARGSGYDLDYWGQGTLVSVSS

SEQ ID NO: 189 - protein, 128 aa, Artificial Sequence
  source: /note=“Description of Artificial Sequence Synthetic polypeptide”
EVQLVESGGGLVQPGGSLRLSCAASGFTFSSFWMTWVRQAPGKGLEWVANIKQDGSEKYYVDSVKGRFTISRDNAKNSLYLQMNSLRAEDTAVYYCARDPGRTMVRGGIRYYYGMDVWGQGTTVTVSS

SEQ ID NO: 190 - protein, 122 aa, Artificial Sequence
  source: /note=“Description of Artificial Sequence Synthetic polypeptide”
EVKLAESGGGLVQPGGSLRLSCAASGFTFSSHWMNWVRQAPGKGLEWVANIKQDGSDKYYVDSVKGRFTISRDNAKNSLYLQLNSLIAEDTAVYYCARDRGVRPPRGAFDIWGQGTMVTVSS

SEQ ID NO: 191 - protein, 128 aa, Artificial Sequence
  source: /note=“Description of Artificial Sequence Synthetic polypeptide”
QVQLVQSGAEVKKPGASVKVSCKASGYTFNSYGISWVRQAPGQGLEWMGWIRTYNGNTNYAQKLQGRVTMTTDTSTSTAYMELRSLRSDDTAVYYCARDEARIVVAGTTPYYYGMDVWGQGTTVTVSS

SEQ ID NO: 192 - protein, 117 aa, Artificial Sequence
  source: /note=“Description of Artificial Sequence Synthetic polypeptide”
QVQLVESGGGLVQPGGSLRLSCAVSGFTISDHYMSWIRQAPGKGLEWISYISSSGSKIYYADSVKGRFTISRDNAKNSLFLQMNSLRAEDTAVYYCARTRQLVGDYWGQGTLVTVSS

SEQ ID NO: 193 - protein, 124 aa, Artificial Sequence
  source: /note=“Description of Artificial Sequence Synthetic polypeptide”
EVQLVESGGGLVQPGRSLRLSCAASGFTFDNYAMHWVRQAPGKGLEWVSGIRWNSGSIGYADSVKGRFTISRDNAKNSLYLQMNSLRAEDTALYYCAKEGGYSGYRPGPFFDYWGQGTLVTVSS

SEQ ID NO: 194 - protein, 117 aa, Artificial Sequence
  source: /note=“Description of Artificial Sequence Synthetic polypeptide”
QVQLVQSGAEVKKPGASVKVSCKASGYTFTNYGISWVRQAPGQGLEWMGWISVYNGHTNYAQKLQGRVTMTTDTSTSTAYMELRSLRSDDTAVYYCARGSGYDFDSWGQGTLVTVSS

SEQ ID NO: 195 - protein, 116 aa, Artificial Sequence
  source: /note=“Description of Artificial Sequence Synthetic polypeptide”
QVQLVQSGAEVKKPGASVKVSCKASRYTFTSYDINWVRQATGQGLEWMGWMNPNSGNTGYAQKFQGRVTMTRNTSTSTAYMELSSLRSEDTAVYYCARVRRFFDYWGQGTLVTVSS

SEQ ID NO: 196 - protein, 117 aa, Artificial Sequence
  source: /note=“Description of Artificial Sequence Synthetic polypeptide”
QVQLVQSGPEVKKPGASVKVSCKASGYTFTNYGISWVRQAPGQGLEWMGWISVYNGNINYAQKLQGRVTMTTDTSTSTAYMDLRSLRSDDTAVYYCARGSGYDFDYWGQGTLVTVSS

SEQ ID NO: 197 - protein, 122 aa, Artificial Sequence
  source: /note=“Description of Artificial Sequence Synthetic polypeptide”
QVQLVQSGAEVKKPGASVKVSCKDSAYTFNRYGISWVRQAPGQGLEWMGWISAYTGNTVYAQKLQGRVTMTTDNSTSTAYMELRSLRSDDTAVYYCARDKSIFGVVRGFDYWGQGTLVTVSS

SEQ ID NO: 198 - protein, 107 aa, Artificial Sequence
  source: /note=“Description of Artificial Sequence Synthetic polypeptide”
AIQMTQSPSSLSASVGDRVTITCRASQGIRNALGWYQQKPGKAPKLLIYAASSLQSGVPSRFSGSGSGTDFTLTFSSLQPEDFATYYCLQDFNYPYTFGQGTKLEIK

SEQ ID NO: 199 - protein, 107 aa, Artificial Sequence
  source: /note=“Description of Artificial Sequence Synthetic polypeptide”
DIQMTQSPSSVSASVGDRVTISCRASQGVSSWLAWYQQKPGNAPKLLISAASSIQSGVPSRFSGSGSGTDFTLTISSLQPEDFATYYCQQANSFPLTFGGGTKVEIK

SEQ ID NO: 200 - protein, 107 aa, Artificial Sequence
  source: /note=“Description of Artificial Sequence Synthetic polypeptide”
DIQMTQSPSSVSASVGDRVTITCRASQGISSWLAWYQQKPGKAPKLLIYAASSFQSGVPSRFSGSGSGTDFTLTISSLQPEDFATYFCQQANSFPLTFGGGTTVEIK

SEQ ID NO: 201 - protein, 107 aa, Artificial Sequence
  source: /note=“Description of Artificial Sequence Synthetic polypeptide”
DIQMTQSPSSVSASVGDRVTITCRASQDISIWLAWYQQSPGKAPKLLINVASRLQSGVPSRFSGSGSGTDFTLTINSLQPEDFVTYYCQQANSFPITFGQGTRLATK

SEQ ID NO: 202 - protein, 107 aa, Artificial Sequence
  source: /note=“Description of Artificial Sequence Synthetic polypeptide”
DIQLTQSPSFLSASVGDRVTITCWASQGISSYLAWYQQKPGKAPKLLIFAASTLQSGVPSRFSGSGSGTEFTLTISSLQPEDFATYYCQQLNSYPLTFGGGTKVEIR

SEQ ID NO: 203 - protein, 107 aa, Artificial Sequence
  source: /note=“Description of Artificial Sequence Synthetic polypeptide”
EIVMTQSPATLSVSPGERATLSCRASQSVNYNLAWYQHKPGQAPRLLIYGASTRATGIPARFSGSGSGTEFTLTISSLQSEDFAVYYCQQYNNWPLTFGGGTKVEIK

SEQ ID NO: 204 - protein, 107 aa, Artificial Sequence
  source: /note=“Description of Artificial Sequence Synthetic polypeptide”
AIQMTQSSSSLSASVGDRVTITCRASQAIRNALGWYQQKPGKAPKVLIYAASSLQSGIPSRFSGSGSGTDFTLTISSLQPEDFATYYCLQDYDYPYTFGQGTKLEIK

SEQ ID NO: 205 - protein, 107 aa, Artificial Sequence
  source: /note=“Description of Artificial Sequence Synthetic polypeptide”
DIQLTQSPSFLSASVGDRVTITCWASQGIISYLAWYQQKPGKAPKLLIYAASTLHSGVPSRFSGSGSGTEFTLTISSLQPEDFATYYCHQLKSYPITFGQGTRLEIK

SEQ ID NO: 206 - protein, 107 aa, Artificial Sequence
  source: /note=“Description of Artificial Sequence Synthetic polypeptide”
AIQMTQSPSSLSASVGDRVTITCRASQDIRNALGWYQQKPGKAPKLLIYAASSLQSGVPSRFSGSASGTDFTLTISSLQPEDFAAYYCLQDYNYPYTFGQGTKLEIK

SEQ ID NO: 207 - protein, 107 aa, Artificial Sequence
  source: /note=“Description of Artificial Sequence Synthetic polypeptide”
EIVMTQSPVTLSLSPGERATLPCRASQSVSSSLAWYQQKAGQSPRLLIYGASTRATGIPARFSGSGSGTEFTLTISNLQSEDFAVYYCQQYNNWPLTFGGGTKVEIK